(19) United States
(12) Patent Application Publication
(10) Pub. No.: US 2016/0207893 A1
(43) Pub. Date: Jul. 21, 2016

(54) NOVEL CALCIUM MODULATORS

(71) Applicant: Myotherix, Inc., Union City, CA (US)

(72) Inventors: Michael Gerard Kelly, Kelseyville, CA (US); John Kincaid, Hayward, CA (US)

(21) Appl. No.: 14/983,352

(22) Filed: Dec. 29, 2015

Related U.S. Application Data
(60) Provisional application No. 62/098,090, filed on Dec. 30, 2014.

(51) Int. Cl. C07D 281/10; A61K 45/06; C07D 513/04; A61K 31/554

(52) U.S. Cl. CPC C07D 281/10; A61K 31/554; A61K 45/06; C07D 513/04

(57) ABSTRACT

Disclosed are novel calcium modulators having formula I:

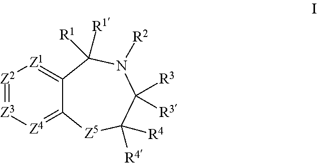

or a pharmaceutically acceptable salt, and including deuterated forms thereof, wherein: Z¹, Z², Z³, Z⁴ Z⁵, R¹, R^{1′}, R², R³, R^{3′} R⁴, and R^{4′}, are as defined throughout the specification; pharmaceutical compositions thereof; and methods of use thereof.

### CROSS-REFERENCES TO RELATED APPLICATIONS

This application claims the benefit of priority to U.S. Provisional Application Ser. No. 62/098,090, filed Dec. 30, 2014, the entire content of which is incorporated herein by reference.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, ORA COMPUTER PROGRAM LISTING APPENDIX SUBMITTED ON A COMPACT DISK

NOT APPLICABLE

### FIELD OF THE INVENTION

The present invention relates to novel calcium modulators. The present invention also relates to novel calcium modulators having a novel combined mechanism of action. The present invention also relates to uses of novel calcium modulators for the treatment of conditions associated with muscle disorders as well as CNS disorders. The present invention also relates to novel pharmaceutical compositions comprising the novel calcium modulators. The present invention also relates to novel pharmaceutical compositions comprising either known or novel calcium modulators in combination with other active agents. The present invention relates to synthetic methods for novel calcium modulators.

### BACKGROUND OF THE INVENTION

Intracellular calcium signaling plays pivotal roles in the regulation of numerous physiological and pathophysiological processes, as virtually all cell types depend in some manner upon the generation of cytoplasmic Ca²⁺ signals to regulate cell function, or to trigger specific responses.

This versatility of Ca²⁺ as an intracellular messenger is derived from varying cytosolic Ca²⁺ concentrations, most of which are maintained by the regulated openings of Ca²⁺-permeable channels expressed in the plasma membrane and in different organelles within cells. For example, a 20,000-fold gradient exists between intracellular free Ca²⁺ concentration and extracellular Ca²⁺ concentration during the resting state (10-100 nM vs. 2 mM), as well as between the intracellular free Ca²⁺ concentration and the free Ca²⁺ concentration in the endoplasmic/sarcoplasmic reticulum (ER/SR). These differences are strictly maintained by Ca²⁺-buffering proteins and a multitude of membrane-specific Ca²⁺ transport and Ca²⁺ modulatory proteins capable of transferring Ca²⁺ from the cytosol into ER/SR or the extracellular environment.

As these cellular proteins (acting both locally and broadly) have adapted to bind Ca²⁺, it is not surprising therefore that dysregulation of Ca²⁺ signaling and impairment of the dynamic equilibrium of Ca²⁺ between the cytosol and endoplasmic/sarcoplasmic reticulum (ER/SR) and the extracellular environment is one of the leading causes of cellular dysfunction in a wide variety of pathological conditions as diverse as cardiac and cardiovascular disease, skin disorders, muscle disorders and diseases of the central nervous system and the like.

While progress has been made in the identification of numerous molecules, biological targets and signaling cascades that are involved in ER/SR Ca²⁺ homeostasis, much is still unknown and needs to be addressed about the causes and consequences of their malfunctioning and their role in human disease. Indeed, calcium leak channels, stretch activated channels, receptor-operated channels, and store operated calcium channels have all been implicated in calcium transport pathways in various cardiac diseases, skeletal muscle diseases and muscular dystrophies.

Voltage-gated calcium channels are a highly conserved family of ion channels that mediate calcium influx in response to membrane depolarization and help regulate intracellular processes associated with cardiac and skeletal muscle contraction, neurotransmission, and gene expression in many different cell types. Their activity is essential to couple electrical signals at a cell surface to physiological events within cells.

Transient receptor potential (TRP) channels are a group of ion channels that serve as cellular sensors for a wide spectrum of physical and chemical stimuli (Clapham 2003, Zheng 2013). They constitute non-selective cation-permeable ion channels, most of which are permeable to Ca²⁺. In skeletal muscle, several isoforms of the TRPC (canonical), TRPV (vanilloid) and TRPM (melastatin) subfamilies are expressed; while TRPC1, TRPC3, TRPC6, TRPV2, TRPV4, TRPM4 and TRPM7 have been found in cultured myoblasts and adult muscles. One such TRP channel in skeletal muscle is TRPC1 (a small-conductance channel of the sarcolemma) that is needed for Ca²⁺ homeostasis during sustained contractile muscle activity; but under certain physiological functions TRP channels are involved in the pathomechanisms of muscle disorders. A growing body of evidence points to dysregulation of Ca²⁺ conducting channels as a key role in the pathomechanism of Duchenne muscular dystrophy (Brinkmeier 2011) anmd other muscle dystrophies. These channels respond to membrane stretch or to depletion of Ca²⁺ stores, while some TRP Channels might also constitute unregulated Ca²⁺ leak channels (Gailly 2012).

Store-operated calcium entry (SOCE), which involves Calcium Release-Activated Calcium (CRAC) channels and their currents (ICRAC), is a process in cellular physiology that controls such diverse functions such as, but not limited to, refilling of intracellular Ca²⁺ stores (Putney et al., 1993), activation of enzymatic activity (Fagan et al., 2000), gene transcription (Lewis, 2001), cell proliferation (Nunez et al., 2006), release of cytokines (Winslow et al., 2003) and calcium homeostasis. In some nonexcitable cells, SOC influx occurs through calcium release-activated calcium (CRAC) channels, a type of SOC channel. Two major components of SOCE have been identified; STIM (a type one single trans-membrane protein resides mainly at the endoplasmic reticulum membrane) and Orai (a four trans-membrane protein localized at the plasma membrane). The depletion of ER calcium is detected by STIM, and results in the expose an important Orai binding domain called CRAC Activating Domain (CAD). Direct binding of CAD to the intracellular domains of Orai results in oligomerization of STIM and Orai and activation of SOCE. These clusters of STIM and Orai form a series of puncta on the plasma membrane that are only visible after depletion of ER and activation of SOCE, and during this stage, the influx of Ca²⁺ can be visualized and measured using colorimetric dyes and electrophysiological techniques.

Inositol 1,4,5-trisphosphate (IP3) receptors are a form of ligand-gated ion channels that are activated by cytosolic Ca²⁺ and IP3. They are localized to intracellular membranes, such as the endoplasmic reticulum, and mediate the mobilization of intracellular Ca²⁺ stores and represent a dominant second messenger leading to the release of Ca²⁺ from intracellular store sites.

The ryanodine receptor (RyR) is a large transmembrane SR/ER Ca²⁺ channel that regulates and controls Ca²⁺ release from the SR/ER during Ca²⁺ signaling events, including excitation-contraction (EC) coupling in contractile tissue.

RyRs are modulated directly or indirectly by the dihydropyridine receptor (Cav1.1/Cav1.2), and by various ions, small molecules as well as other accessory and regulatory proteins, e.g., Ca²⁺, Mg²⁺ ATP, protein kinase A (PKA), FK506-binding proteins (FKBP12 and FKBP12.6), calmodulin (CaM), Ca²⁺/calmodulin-dependent protein kinase II (CaMKII), protein phosphatases PP1 and PP2, calsequestrin, triadin, and junctin (Smith 1986; Tanabe et al. 1990; Ikemoto et al. 1991; Sabbadini et al. 1992; Wang and Best 1992; Brillantes et al. 1994; Chen and MacLennan 1994; Yang et al. 1994; Ma et al. 1995; Mayrleitner et al. 1995; Tripathy et al. 1995; Timerman et al. 1996; Nakai et al. 1998; Moore et al. 1999; Rodney et al. 2000; Carter et al., 2006).

It has been proposed that the association of FKBP to the RyR complex helps stabilize channel function and facilitate coupled gating between neighboring RyRs to prevent abnormal Ca²⁺ leak (or abnormal activation of the channel) by stabilizing the channel's closed state. (Marx et al., 2001). This interpretation has been strongly contested by other researchers that observed no functional consequences of FKBP-dissociation on RyR coupled gating (Hu et al., 2005; Hunt et al., 2007; Oda et al., 2015).

RyR1 is the most thoroughly examined isoform because of its high expression levels in skeletal muscle; it is located in the junctional region of the terminal SR (Franzini-Armstrong and Nunzi 1983). Its primary function is to mediate excitation-contraction coupling, which it does by releasing calcium from the sarcoplasmic reticulum into the cytosol in response to motor neuron-mediated stimulation at the neuromuscular junction (Dulhunty, 2006).

Mutations in the RyR1 gene underlie several debilitating and/or life-threatening muscle diseases including malignant hyperthermia (MH) (MacLennan et al. 1990), heat/exercise induced exertional rhabdomyolysis (Capacchione et al. 2010), central core disease (CCD) (Zhang et al. 1993), multiminicore disease (Ferreiro et al. 2002), ophthalmoplegia (Shaaban et al., 2013), late-onset axial myopathy (Loseth et al., 2013) and atypical periodic paralyses (Zhou et al. 2010). Overall, RyR1-related myopathies are the most common congenital myopathy (Amburgey et al., 2011) and are probably the second-most common group of muscle diseases in childhood (Norwood et al., 2009). Approximately 300 mutations have been identified and linked to diseases associated with RyR (e.g. Jungbluth et al., 2012; Klein et al., 2012).

The predominant form of RyR in cardiac muscle is RyR2 (Nakai et al. 1990; Otsu et al. 1990), and it plays a pivotal role in EC coupling and therefore cardiac muscle contraction. Abnormal SR Ca²⁺ handling attributable to defective RyR2 function is a well-known cause of ventricular tachyarrhythmias and sudden death (Ho et al., 1989; Liu et al., 2002). Naturally occurring RyR2 mutations have been linked to CPVT and catecholaminergic idiopathic ventricular fibrillation (Li et al., 2002; Kong et al., 2008; Jiang et al., 2004. More than 150 disease-associated RyR2 mutations have been identified to date (Jiang et al., 2004 and 2005).

Controversy still exists around an understanding of the mechanistic and functional consequences of the relationship between RyR and its myriad of regulatory proteins, co-factors as well as its post translational modifications. And not being bound by any particular theory or mechanism, there is an urgent need for new therapeutic agents that can modulate the activity of Ca²⁺ modulating proteins to treat human disease.

### Nitric Oxide and Muscle Disease

Nitric oxide (NO), also known as ‘endothelium-derived relaxing factor’ is a powerful vasodilator with a short half-life of less than one minute. In the blood for example, NO disappears within seconds because it binds and reacts with haemoglobin.

In endothelial cells, it is biosynthesized from the amino acid L-arginine, by the constitutive calcium-calmodulin-dependent enzyme nitric oxide synthase (NOS). This heme-containing oxygenase catalyzes a five-electron oxidation from one of the basic guanidino nitrogen atoms of L-arginine in the presence of multiple cofactors (NADPH) and oxygen (Palmer et al., 1988). The charge neutrality and its high diffusion capacity are hallmarks that characterize NO bioactivity.

NO is an endogenous cell-signaling molecule of basic importance in physiology and there is a significant body of evidence that certain diseases are related to a deficiency in the production of NO (Lima et al., 2010). It is known that NO plays multiple physiological roles in regulating numerous and diverse organ functions, and defects in the NO pathway lead to the development of many different pathological conditions such as (but not limited to) hypertension, atherosclerosis, coronary artery diseases, cardiac failure, pulmonary hypertension, stroke, impotence, muscle disorders, muscular dystrophies, muscle fatigue, vascular complications in diabetes mellitus, gastrointestinal ulcers, asthma, and other central and peripheral nervous system disorders (De Palma and Clementi, 2012; Nisoll and Carruba, 2006).

Over the last two decades, NO has been firmly established as a novel mediator of multiple biological processes, ranging from vascular control to long-term memory, from tissue inflammation to penile erection. In more recent years however, skeletal muscle has emerged as the cornerstone of NO function and redox-related signaling in biology (De Palma and Clementi, 2012). All major NOS isoforms are expressed in skeletal muscles of all mammals, including a muscle-specific splice variant of neuronal-type (n)NOS, The expression and localization of various NOS isoforms are dependent on the species and the type of muscle fiber and is influenced by age, developmental stage and disease. Muscle NOS localization and activity are regulated by a number of factors such as protein-protein interactions and co- and/or post-translational modifications. Because of its very short half-life, subcellular compartmentalization of the NOS's enables discrete and distinct functions that are mediated by increases in cGMP and by S-nitrosylation of proteins. Skeletal muscle functions regulated by NO include force production, autoregulation of blood flow, myocyte differentiation, respiration, activation of satellite cells and release of myotrophic factors and glucose homeostasis. In fact, NO mediates satellite cell activation, including morphological hypertrophy and decreased adhesion in the fiber-lamina complex in less than one minute after injury. (Anderson, 2000; Froehner et al., 2015).

Within muscle cells, it has been shown that sarcolemma-localized nNOS is required to maintain activity in mice after mild exercise (Kobayashi et al., 2008). In human DMD muscle (as well as murine models of muscular dystrophies such as the mdx mouse model of DMD or alpha-sarcoglycan null mouse model of Limb Girdle muscular dystrophy), nNOS is absent from the sarcolemma leading to paradoxical exercise-induced vasoconstriction and resulting functional ischemia which contributes to the ongoing muscle damage (Sander et al., 2000, Chang et al., 1996, Chao et al., 1996). In DMD, the loss of dystrophin therefore destabilizes the sarcolemma in multiple ways, rendering muscle fibers susceptible to physical damage with repeated contraction.

NO derived from nNOSμ plays a critical role in the physiology of skeletal muscle, regulating force generation, muscle mass, fatigue, muscle repair from injury, oxidative stress and blood flow. During use and exercise, NO rapidly increases blood flow in contracting muscles to accommodate the elevated metabolic demands of the tissue, and therefore, the loss of nNOSμ is believed to contribute significantly to the dystrophic pathology. Its aberrant regulation and relocalization may contribute to degeneration of muscle fibers in DMD (as well as other muscle diseases), and may have important implications for both pathophysiology as well as the possible treatment of muscle diseases. Manipulating NO levels in muscle therefore may represent an important strategy for treatment of muscular dystrophy (Stamler and Meissner, 2001).

Functional observations have provided direct evidence of altered intestinal motility in mdx mice (the murine model of DMD) (Mancinelli et al., 1995, Mule et al., 2010), in addition to severe disorders of neuronal L-arginine/NO pathways in the mdx colon (Mule 1999, 2001a, 2001b, Serio 2001). It has been demonstrated that iNOS is expressed and active in smooth muscle cells of normal mice and that iNOS is defective in mdx mice (Vannucchi et al., 2004). This altered activity is thought to underpin the motility disorders observed in the colon of mdx mice and, from a clinical point of view, the impairment of intestinal function in dystrophic patients (Backhouse et al., 2006, Fois 1997, Staiano et al., 1996). It has been demonstrated that NO regenerates the normal colonic peristaltic activity in mdx dystrophic mouse (Azzena and Mancinelli, 1999).

Organic nitrates are proven medicinal substances, used to treat dysfunctions of the circulatory system by improving the oxygen supply to the heart via coronary dilatation. Given acutely, organic nitrates are excellent agents for the treatment of stable-effort angina, unstable angina, in patients with acute myocardial infarction and in patients with chronic congestive heart failure. However, the chronic efficacy of nitrates is blunted because of the development of early nitrate tolerance (Elkayam et al., 1987). Organic nitrates and nitrites (such as glyceryl trinitrate, isosorbide dinitrate and amyl nitrite and the like) release NO and activate the same metabolic pathway of endogenous NO (Torfgard and Ahlner 1994) and thus exhibit all its biological properties. Nevertheless, because of their short half-life, which gives a rapid and massive release of NO, their use is substantially limited to those pathological situations requiring a rapid and powerful vasorelaxing effect. It is known that the typical organic nitrates employed in therapy, such as glycerol trinitrate, isosorbide dinitrate or isosorbide 5-mononitrate and the like, display, on continuous intake and within a short period of time, a distinct attenuation of their effect known as “nitrate tolerance” or tachyphylaxis. This is not a recent phenomenon, in 1888 for example, a case of nitroglycerine tolerance in an individual who required 20 grains of pure nitroglycerine to achieve the same hypotensive effect as induced by the initial dose of 1/100 grain was reported, an observation that became a common problem in clinical practice (Stewart, 1888). Nitrate tolerance develops despite an elevation in the drug plasma concentration reflecting a decrease in vascular sensitivity to previously therapeutic levels, and can generally be prevented or reduced by inclusion of a nitrate free period in the dosing schedule. Nitrate-tolerant individuals are generally more susceptible to enhanced vasoconstriction whenever the plasma nitrate concentration is allowed to fall, the so-called rebound effect. This is reflected by increased sensitivity to a number of circulating vasoconstrictor substances such as catecholamines and angiotensin II. This nitrate tolerance and the other side-effects have restricted the clinical use and effectiveness of nitrates. Therefore, NO donor compounds which can produce NO for extended periods of time and do not give rise to nitrate tolerance or tachyphylaxis are needed. (Rutherford, 1995; Thadani, 1997)

The discovery of a role of NO in myogenesis and muscle repair and, thus, the possibility of using NO-based approaches as a treatment for muscular dystrophies and other muscle diseases opens novel perspective for NO as a therapeutic molecule beyond cardiovascular disorders, which have been to date, the only widely recognized field of application of NO donors in humans.

In anticipation of the need for improved NO drugs to treat human disorders, it has been shown that an amelioration of the dystrophic phenotype had been observed in neuronal NO-synthase transgenic mice (Wehling-Henricks et al., 2001). Similarly, it has been demonstrated that cardiomyopathy in dystrophin-deficient hearts is prevented by expression of a neuronal nitric oxide synthase transgene in the myocardium. The expression of the transgene prevented the progressive ventricular fibrosis of mdx mice and greatly reduced myocarditis (Wehling-Henricks et al., 2005).

The double knockout mouse for utrophin and dystrophin (utr^{-/-}/mdx) has been proposed to be a better model of DMD than the mdx mouse because the former displays more similar muscle pathology to that of the DMD patients. Mice deficient for both dystrophin and utrophin show a severe progressive muscular dystrophy that result in premature death (Capote et al., 2010, Deconinck et al., 1997). It has been shown that survival of dystrophin/utrophin double-knockout (Dko) mice was significantly increased by muscle-specific expression of a nNOS transgene. Dko mice expressing the transgene (nNOS TG+/dko) experienced delayed onset of mortality and increased life-span (Wehling-Henricks et al., 2011).

The therapeutic potential of treatments based on the administration of the amino acid L-arginine (a metabolic precursor of NO), or molsidomine (a long acting vasodilating drug that releases NO upon metabolism) have been investigated. Molsidomine is an established drug for the treatment of coronary heart disease, which acts via the metabolite SIN-1 through liberation of NO (Reden 1990). Molsidomine was reported to decrease inflammatory cell infiltrate in the α-sarcoglican-null mice (a model for the limb girdle muscular dystrophy 2D) (Zordan et al., 2013). Although some amelioration of muscle morphology was observed, and in one study creatine kinase levels were reduced, those treatments did not yield recovery of muscle function, nor did they improve animal motility tests and demonstrate that an improvement over Molsidomine is urgently needed for a more effective therapy (Benabdellah et al., 2009; Zordan et al., 2013). The inorganic vasodilator and NO donor, sodium nitroprusside (SNP), has been shown to act as an endogenous anti-inflammatory molecule during ongoing muscle inflammation (Liu et al., 2015). Its short biological half-life (<2 minutes) and lack of oral activity underline the need more effective therapies than SNP to target and effectively treat muscle diseases.

Various forms of NO-donating compounds have been described in recent years by simply conjugating NO-donating moieties to existing well-characterized and well-known drugs (e.g. naproxen, aspirin, acetaminophen, prednisolone, captopril, statins (e.g. prevastatin, fluvastatin, atorvastatin and the like), β-blockers, 1,4-dihydropyridine Ca²⁺ antagonists (nifedipine, amlodipine and the like), ATP-sensitive K-channel opener (nicorandil etc.), angiotensin converting enzyme inhibitors (captopril, enalapril and the like), angiotensin II receptor blockers (losartan, telmisartan and the like), antidiabetic agents (glibenclamide) and gabapentin etc.). This has generated new agents that preserve the activity of the known drug, enriched with NO activity, with a specific aim in many cases of improving the safety and tolerability profile of the existing well-known drugs (Bolla et al., 2005; Martelli et al., 2009; Gasco et al., 2008), particularly the cardiovascular and GI safety profile of the non steroidal anti-inflammatory drugs (NSAID's).

The use of NO-donating non steroidal anti-inflammatory drugs in dystrophic mouse models have been reported by a number of investigators. By way of examples, naproxcinod (an NO-donating form of naproxen) was administered to mdx mice (a murine model of DMD) for nine months and found to improve hindlimb grip strength as well as improve heart function (Uaesoontrachoon et al., 2014). A similar compound, NCX 320 (an NO-donating form of the NSAID ibuprofen), was administered to the alpha-sarcoglycan null mice for 8 months. NCX 320 mitigated muscle damage, significantly reduced serum creatine kinase activity, reduced the number of necrotic fibers and inflammatory infiltrates. A further similar compound, HCT 1026 (an NO-donating form of the NSAID flurbiprofen), was administered to two murine models for limb girdle and Duchenne muscular dystrophies (alpha-sarcoglycan-null and mdx mice). HCT 1026 was shown to reduce inflammation, preventing muscle damage, and preserving the number and function of satellite cells (Brunelli et al., 2007).

Most notably, and in reference to the above studies with NO-donating NSAID's, a most recent study compared the administration of naproxcinod and naproxen separately to the mdx mouse model of Duchenne for six months and showed that naproxcinod treatment significantly ameliorated skeletal muscle force and resistance to fatigue in sedentary as well as exercised mice, reduced inflammatory infiltrates and fibrosis deposition in both cardiac and diaphragm muscles. Conversely, an equimolar dose of naproxen showed no effects on fibrosis and improved muscle function only in sedentary mice, while the beneficial effects in exercised mice were lost demonstrating a limited and short-term effect with the NSAID (Miglietta et al., 2015).

Most notably, and further to the above discussion, the results from a clinical study, examining the safety and efficacy of long term administration (12 months) of a combination of the NSAID ibuprofen with the NO donor isosorbide dinitrate to 71 patients with various muscular dystrophies (DMD, Becker MD, Limb-Girdle MD) failed to demonstrate significant benefits like those seen in the murine models of dystrophy (D'Angelo et al., 2012). It is clear therefore that an improvement over that produced by NO-donating NSAID's as well as an NO-donor in combination with a NSAID is urgently needed in order to provide a more effective therapy to patients in need.

The potential use of NO-donating molecules in treating complex diseases associated with muscle disorders, particularly for example muscular dystrophy, brings about important considerations, the most significant of which is that the state of the current art to elicit NO donation alone appears insufficient to yield a full therapeutic benefit. There is an important and urgent unmet medical need therefore to improve upon the beneficial effects of NO in myogenesis and muscle repair beyond that attainable by the use of either organic nitrates, a NO-donor in combination with a NSAID, or an NO-donating NSAID.

Many biological effects of NO and NO-derived molecules are mediated through cGMP-independent pathways via post-translational modification of proteins such as S-nitrosylation of target proteins, receptors, ion channels, enzymes, and transcription factors among others (Handy and Loscalzo, 2006; Corpas et al., 2008).

NO can lead to thiol nitrosylation of cysteine residues termed S-nitrosylation and tyrosine nitration. These modifications have an impact on protein structure and function and are largely generated through the excessive production of NO which occurs through overactivation of nNOS or induction of iNOS as often found in disease states.

Transient receptor potential (TRP) channels are a group of ion channels that serve as cellular sensors for a wide spectrum of physical and chemical stimuli (Clapham 2003, Zheng 2013). Recombinant TRPC and TRPV families induce entry of Ca²⁺ into cells in response to NO. Cytoplasmically accessible Cys residues (553 and nearby 558 on TRPC5) are nitrosylation sites which mediate NO sensitivity of these ion channels. Nitric oxide activates TRP channels by cysteine S-nitrosylation and increases Ca²⁺ entry or Ca²⁺ leak (Yoshida et al., 2006, Voolstra and Huber 2014). Modification of components of the SOC pathway (STIM and Orai) by NO not been fully investigated. Nevertheless, STIM1 proteins possess several cysteines residues that could be targets for modification. Similarly, all three Orai isoforms possess predicted extracellular and intracellular cysteines (Trebak et al., 2010)

RyR's contain multiple cysteine residues (>50 cysteine residues per RyR1 subunit) that can be modified at physiological pH by S-nitrosylation (Xu et al., 1998; Sun et al., 2001; Aracena et al., 2003; Sun et al., 2003). cGMP-independent, NO-mediated regulation of RyR's increase the channels activity in vesicles and in single channel measurements (Xu et al., 1998), and exogenous S-nitrosylation of RyR1 has been shown to reduce the affinity of FKPB12 binding to SR triads (Aracena et al., 2005).

It has been demonstrated that iNOS co-localizes with RyR1 in skeletal muscles and that the dystrophic muscle from mdx mice had increased levels of RyR1 S-nitrosylation which correlated with depletion of FKPB12 from the RyR1 complex leading to disruption of channel function. Exogenous S-nitrosylation, using NO-donors, was shown to result in depletion of FKBP12 from immunoprecipitated RyR1. These RyR1 channels demonstrated a Ca²⁺ leak due to depletion of FKBP12 induced by S-nitrosylation (Bellinger et al., 2009).

RyR1 from aged mice have been shown to be oxidized, cysteine-nitrosylated, and depleted of the channel stabilizing subunit FKBP12, when compared to RyR1 from younger mice. This RyR1 channel complex remodeling resulted in “leaky” channels with increased open probability leading to intracellular calcium leak in skeletal muscle (Andersson et al., 2011).

Skeletal muscle weakness is also a prominent clinical feature in patients with rheumatoid arthritis (RA). It has been found that (arthritis-induced) muscle weakness in collagen-induced arthritis mice as well as in patients with RA, is linked to nitrosative modifications of the RyR1 protein complex and actin. This is driven by increased nNOS associated with RyR1 and progressively increasing Ca²⁺ activation (Yamada et al., 2014). RyR1 S-nitrosylation appears to underpin many components of sarcopenia, as uncontrolled Ca²⁺ release by RyR1 from the SR causes activation of Ca²⁺-dependent proteases, and reduced abilities of skeletal muscles to adapt to physical exercise stimuli (Suhr et al., 2013).

It is apparent therefore, that over the past two decades, the evolving art clearly teaches that NO, through a post-translational modification of cysteine(s), can play a detrimental role in RyR function (reducing FKBP and calmodulin binding, reduced EC-coupling, increased skeletal muscle breakdown) as well as other SOC ion channels involved in Ca²⁺ release. This leads to altered Ca²⁺ homeostasis and increased open probability of the channel leading to an intracellular calcium leak.

WO 2007/0049752 discloses 1,4-benzothiazepines for the use of modulating RyR receptors for treating and preventing disorders associated with RyR modulation, such as CNS and muscular disorders. The compounds disclosed in the 752 patent application contain only 1,4-benzothiazipines and the disclosed biological use for these benzothiazepines is specific for just modulating RyR receptors.

WO 2008/144483 discloses various 1,4-benzoxazepines, benzazepines, and 1,4-benzothiazepines for the use of modulating RyR receptors for treating and preventing disorders associated with RyR modulation, such as CNS and muscular disorders. The compounds disclosed in the '483 patent application contain only 1,4-benzoxazepines, benzazepines and 1,4-benzothiazepines, and the disclosed biological use for these compounds is specific for modulating RyR receptors.

WO 2013/156505 discloses a very narrow group of 1,4-benzothiazepines for the use of modulating RyR receptors for treating and preventing disorders associated with RyR modulation, such as CNS and muscular disorders. The compounds disclosed in the '505 patent application contain only 1,4-benzothiazipines and the described biological use of these benzothiazepines is specific for modulating RyR receptors.

Accordingly, there is a long felt and unmet need for new and improved agents that modulate calcium function as well as more effective methods for administering calcium modulators, that overcome the deficiencies and problems that are described and taught in the art. More specifically, there is a long felt and unmet need for new and novel calcium modulators for use in the treatment of CNS and muscular disorders. There is also an important unmet and urgent need therefore to improve upon the beneficial effects of NO in myogenesis and muscle repair beyond that attainable by the use of organic nitrates.

### REFERENCES

WO 2007/0049752, NOVEL AGENTS FOR PREVENTING AND TREATING DISORDERS INVOLVING MODULATION OF THE RYR RECEPTORS.
WO 2008/144483, AGENTS FOR TREATING DISORDERS INVOLVING MODULATING OF RYANODINE RECEPTORS
WO 2013/156505, AGENTS FOR TREATING DISORDERS INVOLVING MODULATING OF RYANODINE RECEPTORS
Zorzato F, Fuji J, Otsu K, Phillips M, Green N M, Lai F A, Meissner G, MacLennan D H 1990. Molecular cloning of cDNA encoding human and rabbit forms of the Ca²⁺ release channel (ryanodine receptor) of skeletal muscle sarcoplasmic reticulum. J Biol Chem 265: 2244-2256
Otsu K, Willard H F, Khanna V K, Zorzato F, Green N M, MacLennan D H 1990. Molecular cloning of cDNA encoding the Ca²⁺ release channel (ryanodine receptor) of rabbit cardiac muscle sarcoplasmic reticulum. J Biol Chem 265: 13472-13483
Yoshida T et al., Nitric oxide activates TRP channels by cysteine S-nitrosylation, Nat Chem Biol. 2006 November; 2(11):596-607.
Trebak M. et al., Interplay Between Calcium and Reactive Oxygen/Nitrogen Species: An Essential Paradigm for Vascular Smooth Muscle Signaling, Antioxid Redox Signal. 2010 Mar. 1; 12(5): 657-674.
Clapham D E, TRP channels as cellular sensors, Nature. 2003 Dec. 4; 426(6966):517-24.
Zheng J., Molecular mechanism of TRP channels, Compr Physiol. 2013 January; 3(1):221-42.
Gailly P, TRP channels in normal and dystrophic skeletal muscle, Curr Opin Pharmacol. 2012 June; 12(3):326-34.
Putney J W Jr., Excitement about calcium signaling in inexcitable cells, Science. 1993 Oct. 29; 262(5134):676-8.
Fagan K A, Graf R A, Tolman S, Schaack J, Cooper D M, Regulation of a Ca²⁺-sensitive adenylyl cyclase in an excitable cell. Role of voltage-gated versus capacitative Ca²⁺ entry, J Biol Chem. 2000 Dec. 22; 275(51):40187-94.
Voolstra O, Huber A, Post-Translational Modifications of TRP Channels, Cells. 2014 Apr. 8; 3(2):258-87.
Winslow M M, Neilson J R, Crabtree G R, Calcium signalling in lymphocytes, Curr Opin Immunol. 2003 June; 15(3):299-307.
Núñez, Lucia et al. “Cell Proliferation Depends on Mitochondrial Ca²⁺ Uptake: Inhibition by Salicylate.” The Journal of Physiology 571. Pt 1 (2006): 57-73.
Lewis R S, Calcium signaling mechanisms in T lymphocytes, Annu Rev Immunol. 2001; 19:497-521.
Fagan, K. A., K. E. Smith, and D. M. Cooper. 2000. Regulation of the Ca²⁺-inhibitable adenylyl cyclase type VI by capacitative Ca²⁺ entry requires localization in cholesterol-rich domains. J. Biol. Chem. 275:26530-26537.
Carter S, Colyer J, Sitsapesan R, Maximum Phosphorylation of the Cardiac Ryanodine Receptor at Serine-2809 by Protein Kinase A Produces Unique Modifications to Channel Gating and Conductance Not Observed at Lower Levels of Phosphorylation, Circ Res. 2006; 98:16506-1513.
Parekh A B, Putney J W, Jr. Store-operated calc ium channels. Physiol Rev. 2005; 85:757-810.
Capacchione J F, Sambuughin N, Bina S, Mulligan L P, Lawson T D, Muldoon S M., Exertional rhabdomyolysis and malignant hyperthermia in a patient with ryanodine receptor type 1 gene, L-type calcium channel alpha-1 subunit gene, and calsequestrin-1 gene polymorphisms, Anesthesiology. 2010 January; 112(1):239-44.
Smith J, Coronado R, Meissner G 1986. Single channel measurements of the calcium release channel from skeletal muscle sarcoplasmic reticulum. Activation by Ca²⁺ and ATP and modulation by Mg²⁺. J Gen Physiol 88: 573-588
Chen S R, MacLennan D H 1994. Identification of calmodulin-, Ca²⁺-, and ruthenium red-binding domains in the Ca²⁺ release channel (ryanodine receptor) of rabbit skeletal muscle sarcoplasmic reticulum. J Biol Chem 269: 22698-22704
Nakai J, Sekiguchi N, Rando T A, Allen P D, Beam K G 1998. Two regions of the ryanodine receptor involved in coupling with L-type Ca²⁺ channels. J Biol Chem 273: 13403-13406
Nakai J, Imagawa T, Hakamat Y, Shigekawa M, Takeshima H, Numa S., Primary structure and functional expression from cDNA of the cardiac ryanodine receptor/calcium release channel, FEBS Lett. 1990 Oct. 1; 271(1-2):169-77.
Zhang Y., Chen H. S., Khanna V. K., De Leon S., Phillips M. S., Schappert K., Britt B. A., Browell A. K., MacLennan D. H. 1993. A mutation in the human ryanodine receptor gene associated with central core disease. Nat Genet 5: 46-50
Ferreiro A, Quijano-Roy S, Pichereau C, Moghadaszadeh B, Goemans N, Bönnemann C, Jungbluth H, Straub V, Villanova M, Leroy J P, Romero N B, Martin J J, Muntoni F, Voit T, Estournet B, Richard P, Fardeau M, Guicheney P., Mutations of the selenoprotein N gene, which is implicated in rigid spine muscular dystrophy, cause the classical phenotype of multiminicore disease: reassessing the nosology of early-onset myopathies, Am J Hum Genet. 2002 October; 71(4): 739-49.
Løseth S, Voermans N C, Torbergsen T, Lillis S, Jonsrud C, Lindal S, Kamsteeg E J, Lammens M, Broman M, Dekomien G, Maddison P, Muntoni F, Sewry C, Radunovic A, de Visser M, Straub V, van Engelen B, Jungbluth H., A novel late-onset axial myopathy associated with mutations in the skeletal muscle ryanodine receptor (RYR1) gene, J Neurol. 2013 June; 260(6):1504-10.
Shaaban S, Ramos-Platt L, Gilles F H, Chan W M, Andrews C, De Girolami U, Demer J, Engle E C, RYR1 mutations as a cause of ophthalmoplegia, facial weakness, and malignant hyperthermia, JAMA Ophthalmol. 2013 December; 131(12):1532-40.
Zhou H, Lillis S, Loy R E, Ghassemi F, Rose M R, Norwood F, Mills K, Al-Sarraj S, Lane R J, Feng L, Matthews E, Sewry C A, Abbs S, Buk S, Hanna M, Treves S, Dirksen R T, Meissner G, Muntoni F, Jungbluth H., Multi-minicore disease and atypical periodic paralysis associated with novel mutations in the skeletal muscle ryanodine receptor (RYR1) gene, Neuromuscul Disord. 2010 March; 20(3):166-73.
Liu Z, Zhang J, Li P, Chen S R & Wagenknecht T. (2002). Three-dimensional reconstruction of the recombinant type 2 ryanodine receptor and localization of its divergent region 1. J Biol Chem 277, 46712-46719.
Hu X F, Liang X, Chen K Y, Zhu P H & Hu J. (2005). Depletion of FKBP does not affect the interaction between isolated ryanodine receptors. Biochem Biophys Res Commun 336, 128-133.
Jiang D, Wang R, Xiao B, Kong H, Hunt D J, Choi P, Zhang L & Chen S R., Enhanced store overload-induced Ca2+ release and channel sensitivity to luminal Ca2+ activation are common defects of RyR2 mutations linked to ventricular tachycardia and sudden death. Circ Res 97, 1173-1181 (2005).
Shaaban, S., et al., RYR1 Mutations as a Cause of Ophthalmoplegia FacialWeakness, and Malignant Hyperthermia, JAMA Ophthalmol. 2013; 131(12):1532-1540.
Loseth, S., et al., A novel late-onset axial myopathy associated with mutations in the skeletal muscle ryanodine receptor (RYR1) gene, J Neurol (2013) 260:1504-1510
Jungbluth, H., et al., 182nd ENMC International Workshop: RYR1-related myopathies, 15-17th April 2011, Naarden, The Netherlands. Neuromuscular Disorders 22 (2012) 453-462.
Klein A, Lillis S, Munteanu I, Scoto M, Zhou H, Quinlivan R, Straub V, Manzur A Y, Roper H, Jeannet P Y, Rakowicz W, Jones D H, Jensen U B, Wraige E, Trump N, Schara U, Lochmuller H, Sarkozy A, Kingston H, Norwood F, Damian M, Kirschner J, Longman C, Roberts M, Auer-Grumbach M, Hughes I, Bushby K, Sewry C, Robb S, Abbs S, Jungbluth H, Muntoni F., Clinical and genetic findings in a large cohort of patients with ryanodine receptor 1 gene-associated myopathies, Hum Mutat. 2012 June; 33(6):981-8.
Amburgey K, McNamara N, Bennett L R, McCormick M E, Acsadi G, Dowling J J. Prevalence of congenital myopathies in a representative pediatric united states population. Ann Neurol 2011; 70: 662-5.
Norwood F L, Harling C, Chinnery P F, Eagle M, Bushby K, Straub V. Prevalence of genetic muscle disease in Northern England: in-depth analysis of a muscle clinic population. Brain 2009; 132 (Pt 11): 3175-86.
Dulhunty A F. Excitation-contraction coupling from the 1950s into the new millennium. Clin Exp Pharmacol Physiol 2006; 33: 763-72.
Ikemoto, T., Iino, M., and Endo, M. (1995) Enhancing effect of calmodulin on Ca2+-induced Ca2+ release in the sarcoplasmic reticulum of rabbit skeletal muscle fibres. J. Physiol. 487 (Part3), 573-582.
Tripathy, A., Xu, L., Mann, G., and Meissner, G. (1995) Calmodulin activation and inhibition of skeletal muscle Ca2+ release channel (ryanodine receptor). Biophys. J. 69, 106-119.
Ikemoto N, Antoniu B, Kang J J, Meszaros L G, Ronj at M 1991. Intravesicular calcium transient during calcium release from sarcoplasmic reticulum. Biochemistry 30: 5230-5237
Ho S N, Hunt H D, Horton R M, Pullen J K, Pease L R. Site-directed mutagenesis by overlap extension using the polymerase chain reaction. Gene. 1989; 77:51-59.
Liu Z, Zhang J, Li P, Chen S R, Wagenknecht T., Three-dimensional reconstruction of the recombinant type 2 ryanodine receptor and localization of its divergent region 1. J Biol Chem., 2002; 240:24.
Sabbadini R A, Betto R, Teresi A, Fachechi-Cassano G, Salviati G 1992. The effects of sphingosine on sarcoplasmic reticulum membrane calcium release. J Biol Chemo., 267: 15475-15484.
Tanabe T, Beam K G, Adams B A, Niidome T, Numa S 1990. Regions of the skeletal muscle dihydropyridine receptor critical for excitation-contraction coupling. Nature 346: 567-569.
Li P, Chen S R. Molecular basis of Ca(2)+ activation of the mouse cardiac Ca(2)+ release channel (ryanodine receptor), J Gen Physiol. 2001; 118:33-44.
Kong H, Jones P P, Koop A, Zhang L, Duff H J, Chen S R. Caffeine induces Ca2+ release by reducing the threshold for luminal Ca2+ activation of the ryanodine receptor, Biochem J. 2008, Sep. 15; 414(3):441-52.
Wang J, Best P M 1992. Inactivation of the sarcoplasmic reticulum calcium channel by protein kinase, Nature 359: 739-741
Yang H C, Reedy M M, Burke C L, Strasburg G M 1994. Calmodulin interaction with the skeletal muscle sarcoplasmic reticulum calcium channel protein. Biochemistry 33: 518-525
Jiang D, Xiao B, Yang D, Wang R, Choi P, Zhang L, Cheng H, Chen S R W. RyR2 mutations linked to ventricular tachycardia and sudden death reduce the threshold for store-overload induced Ca2+ release (SOICR). Proc. Natl. Acad. Sci. U.S.A. 2004; 101:13062-13067.
Jiang D, Wang R, Xiao B, Kong H, Hunt D J, Choi P, Zhang L, Chen S R W. Enhanced store overload-induced Ca2+ release and channel sensitivity to luminal Ca2+ activation are common defects of RyR2 mutations linked to ventricular tachycardia and sudden death. Circ Res. 2005; 97:1173-1181.
Lehnart S. E. et al., Leaky Ca2+ release channel/ryanodine receptor 2 causes seizures and sudden cardiac death in mice, J Clin Invest. 2008 June; 118(6):2230-45.
Bellinger A. M., Reiken S., Carlson C., Mongillo M., Liu X., Rothman L., Matecki S., Lacampagne A., Marks A. R., Hypernitrosylated ryanodine receptor calcium release channels are leaky in dystrophic muscle, Nature Medicine, 2009 15, 325-330.
Brillantes A B, Ondrias K, Scott A, Kobrinsky E, Ondriasova E, Moschella M C, Jayaraman T, Landers M, Ehrlich B E, Marks A R, 1994. Stabilization of calcium release channel (ryanodine receptor) function by FK506-binding protein. Cell 77: 513-523.
Marx S O, Ondrias K, Marks A R., Coupled gating between individual skeletal muscle Ca2+ release channels (ryanodine receptors), Science. 1998 Aug. 7; 281(5378):818-21.
Marx S O, Gaburjakova J, Gaburjakova M, Henrikson C, Ondrias K, Marks A R., Coupled gating between cardiac calcium release channels (ryanodine receptors)., Circ Res. 2001 Jun. 8; 88(11):1151-8.
Andersson D. C., Meli A. C., Reiken S., Betzenhauser M. J., Umanskaya A., Shiomi T., D'Armiento J., and Marks A. R., Leaky ryanodine receptors in β-sarcoglycan deficient mice: a potential common defect in muscular dystrophy Skeletal Muscle 2012, 2:9.
Andersson D. C. et al., Ryanodine Receptor Oxidation Causes Intracellular Calcium Leak and Muscle Weakness in Aging, Cell Metab. 2011 Aug. 3; 14(2): 196-207.
Oda T., et al., Oxidation of ryanodine receptor (RyR) and calmodulin enhance Ca release and pathologically alter, RyR structure and calmodulin affinity. J Mol Cell Cardiol. 2015 August; 85:240-8.
Franzini-Armstrong C, Nunzi G., Junctional feet and particles in the triads of a fast-twitch muscle fibre, J Muscle Res Cell Motil. 1983 April; 4(2):233-52.
Mancinelli R, Tonali P, Servidei S, Azzena G B. Analysis of peristaltic reflex in young mdx dystrophic mice. Neurosci Lett 1995; 192:57-60.
Mulé F., D'Angelo S, Tabacchi G, Amato A, Serio R. Mechanical activity of small and large intestine in normal and mdx mice: a comparative analysis. Neurogastroenterol Motil 1999; 11:133-139.
Mulé F., Serio R. Increased calcium influx is responsible for the sustained mechanical tone in colon from dystrophic (mdx) mice. Gastroenterology 2001; 120:1430-1437.
Mulé F., Vannucchi M G, Corsani L, Serio R, Faussone-Pellegrini M S. Myogenic NOS and endogenous NO production are defective in colon from dystrophic (mdx) mice. Am J Physiol Gastrointest Liver Physiol 2001; 281:G1264-G1270.
Serio R, Bonvissuto F, Mule' F. Altered electrical activity in colonic smooth muscle cells from dystrophic (mdx) mice. Neurogastroenterol Motil 2001; 13:169-175.
Vannucchi M. G., et al., Functional activity and expression of inducible nitric oxide synthase (iNOS) in muscle of the isolated distal colon of mdx mice, Muscle Nerve 29: 795-803, 2004.
Backhouse M., Harding L., Australian and New Zealand Continence Journal, Vol 12, No 3, Spring 2006.
Azzena G B., Mancinelli R., Nitric oxide regenerates the normal colonic peristaltic activity in mdx dystrophic mouse. Neurosci Lett. 1999 Feb. 12; 261(1-2):9-12.
Fois A., Gastrointestinal Disorders in Muscular Dystrophies, Journal of Pediatric Gastroenterology & Nutrition: 1997-Volume 25-Issue-pp 20, 21.
Staiano A., Gastrointestinal motility in children with muscular dystrophy, Journal of Pediatric Gastroenterology & Nutrition: May 1996-Volume 22-Issue 4-p 422.
Mulé F., Amato A., Serio R., Gastric emptying, small intestinal transit and fecal output in dystrophic (mdx) mice, J Physiol Sci (2010) 60:75-79.
Mulé F., D'Angelo S, Tabacchi G, Amato A, Serio R., Mechanical activity of small and large intestine in normal and mdx mice: a comparative analysis, Neurogastroenterol Motil. 1999 April; 11(2):133-9.
Wehling-Henricks M., Tidball J. G., Neuronal Nitric Oxide Synthase-Rescue of Dystrophin/Utrophin Double Knockout Mice does not Require nNOS Localization to the Cell Membrane, PLoS ONE 6(10): e25071 (2011)
Wehling-Henricks M. et al., Cardiomyopathy in dystrophin-deficient hearts is prevented by expression of a neuronal nitric oxide synthase transgene in the myocardium. Hum Molec. Genet., 2005 14: 1921-1933.
Wehling-Henricks M et al., A nitric oxide synthase transgene ameliorates muscular dystrophy in mdx mice. J Cell Biol 155: 123-131, 2001.
Rutherford J D, Nitrate tolerance in angina therapy. How to avoid it, Drugs, 1995, 49(2): 196-9.
Thadani U, Nitrate tolerance, rebound, and their clinical relevance in stable angina pectoris, unstable angina, and heart failure, Cardiovasc Drugs Ther., 1997, 10(6): 735-42.
Stewart D D. Remarkable tolerance to nitroglycerin. Phila Polyclin. 1888; 6:43.
Elkayam U, Kulick D, McIntosh N, Roth A, Hsueh W, Rahimtoola S H., Incidence of early tolerance to hemodynamic effects of continuous infusion of nitroglycerin in patients with coronary artery disease and heart failure, Circulation. 1987 September; 76(3):577-84.
Capote J., DiFranco M., Julio L. Vergara J. L., Excitation-contraction coupling alterations in mdx and utrophin/dystrophin double knockout mice: a comparative study, Am J Physiol Cell Physiol 298: C1077-C1086, 2010.
Deconinck A. E., et al., Utrophin-Dystrophin-Deficient Mice as a Model for Duchenne Muscular Dystrophy, Cell, Vol. 90, 717-727, Aug. 22, 1997.
Palmer R. M. J., Ashton D. S., Moncada S., Vascular endothelial cells synthesize nitric oxide from L-arginine. Nature 1988; 333:664-666.
Anderson J. E., A Role for Nitric Oxide in Muscle Repair: Nitric Oxide-mediated Activation of Muscle Satellite Cells. Mol Biol Cell, 2000, 11(5): 1859-1874.
Kobayashi, Y. M. et al., Sarcolemma-localized nNOS is required to maintain activity after mild exercise. Nature 456, 511-515 (2008).
Sander M. et al., Functional muscle ischemia in neuronal nitric oxide synthase-deficient skeletal muscle of children with Duchenne muscular dystrophy. Proc Natl Acad Sci USA 97: 13818-13823, 2000.
Torfgard K. E., Ahlner J., Mechanisms of action of nitrates. Cardiovasc Drug Ther 1994; 8: 701-17.
Bolla M., Almirante N., Benedini F., Therapeutic Potential of Nitrate Esters of Commonly Used Drugs, Current Topics in Medicinal Chemistry 2005, 5, 707-720.
Martelli A., Breschi M. C., Calderone V., Pharmacodynamic Hybrids Coupling Established Cardiovascular Mechanisms of Action with Additional Nitric Oxide Releasing Properties, Current Pharmaceutical Design, 2009, 15, 614-636.
Benabdellah F., Yu H., Brunelle A., Laprévote o., De La Porte S., MALDI reveals membrane lipid profile reversion in MDX mice, Neurobiol Dis. 2009 November; 36(2):252-8.
Zordan P., Sciorati C., Campana L., Cottone L., Clementi E., Querini P. R., Brunelli S., The nitric oxide-donor molsidomine modulates the innate inflammatory response in a mouse model of muscular dystrophy, Eur J Pharmacol. 2013 Sep. 5; 715(1-3):296-303.
Reden J., Molsidomine, Blood Vessels. 1990; 27(2-5):282-94.
Yamada T, Fedotovskaya O, Cheng A J, et al., Nitrosative modifications of the Ca2+ release complex and actin underlie arthritis-induced muscle weakness, Ann Rheum Dis., May 22, 2014
Uaesoontrachoon K. et al., Long-term treatment with naproxcinod significantly improves skeletal and cardiac disease phenotype in the mdx mouse model of dystrophy, Human Molecular Genetics, 2014, Vol. 23, No. 12, 3239-3249.
Sciorati C. et al., A dual acting compound releasing nitric oxide (NO) and ibuprofen, NCX 320, shows significant therapeutic effects in a mouse model of muscular dystrophy, Pharmacological Research 64 (2011) 210-217.
Brunelli S. et al., Nitric oxide release combined with nonsteroidal antiinflammatory activity prevents muscular dystrophy pathology and enhances stem cell therapy, PNAS, Jan. 2, 2007, vol. 104, no. 1, 264-269.
Chao D. S. et al., Selective Loss of Sarcolemmal Nitric Oxide Synthase in Becker Muscular Dystrophy, J. Exp. Med., Volume 184, August 1996, 609-618.
Chang W-J. et al., Neuronal nitric oxide synthase and dystrophin-deficient muscular dystrophy, Proc. Natl. Acad. Sci. USA, Vol. 93, pp. 9142-9147, August 1996.
Lima B., Forrester M. T., Hess D. T., Stamler J. S., S-Nitrosylation in Cardiovascular Signaling, Circ Res. 2010; 106:633-646.
Mayrleitner M, Chandler R, Schindler H, Fleischer S 1995. Phosphorylation with protein kinases modulates calcium loading of terminal cisternae of sarcoplasmic reticulum from skeletal muscle. Cell Calcium 18: 197-206.
Timerman A P, Onoue H, Xin H-B, Barg S, Copello J, Wiederrecht G, Fleischer S 1996. Selective Binding of FKBP12.6 by the Cardiac Ryanodine Receptor. J Biol Chem 271: 20385-20391.
Miglietta D., De Palma C., Sciorati C., Vergani B., Pisa V., Villa A., Ongini E and Clementi E., Naproxcinod shows significant advantages over naproxen in the mdx model of Duchenne Muscular Dystrophy, Orphanet Journal of Rare Diseases (2015) 10:101.
Ma J, Bhat M B, Zhao J 1995. Rectification of skeletal muscle ryanodine receptor mediated by FK506 binding protein. Biophys J 69: 2398-2404.
D'Angelo M. G. et al., Nitric oxide donor and non steroidal anti inflammatory drugs as a therapy for muscular dystrophies: Evidence from a safety study with pilot efficacy measures in adult dystrophic patients, Pharmacological Research 65 (2012) 472-479.
De Palma C., Clementi E., Nitric Oxide in Myogenesis and Therapeutic Muscle Repair, Mol Neurobiol (2012) 46:682-692.
Froehner S. C. et al., Loss of nNOS inhibits compensatory muscle hypertrophy and exacerbates inflammation and eccentric contraction-induced damage in mdx mice, Human Molecular Genetics, 2015, Vol. 24, No. 2, 492-505.
Kobayashi Y M, Rader E P, Crawford R W, Iyengar N K, Thedens D R, Faulkner J A, Parikh S V, Weiss R M, Chamberlain J S, Moore S A, Campbell K P., Sarcolemma-localized nNOS is required to maintain activity after mild exercise, Nature. 2008 Nov. 27; 456(7221):511-5.
Gasco A., et al., Multitarget drugs: Focus on the N O-donor hybrid drugs, Pure Appl. Chem., Vol. 80, No. 8, pp. 1693-1701, 2008.
Liu X. H. et al., Effects of Nitric Oxide on Notexin-Induced Muscle Inflammatory Responses, Int. J. Biol. Sci. 2015, Vol. 11, 156.
Stamler J. S. and Meissner G., Physiology of Nitric Oxide in Skeletal Muscle,
Physiological Reviews, Vol. 81, No. 1, January 2001.
Nisoll E. and Carruba M. O., Nitric oxide and mitochondrial biogenesis, Journal of Cell Science 119 (14), 2855, (2006).
Handy D. E. and Loscalzo J., Nitric Oxide and Posttranslational Modification of the Vascular Proteome, Arterioscler Thromb Vasc Biol. 2006; 26:1207-1214.
Corpas F. J., del Rio L. A. and Barroso J. B., Post-translational modifications mediated by reactive nitrogen species, Plant Signaling & Behavior 3:5, 301-303; May 2008.
Xu L., Eu J. P., Meissner G., Stamler J. S., Activation of the cardiac calcium release channel (ryanodine receptor) by poly-S-nitrosylation. Science. 1998; 279:234-237.
Sun J., Xin C., Eu J. P., Stamler J. S., Meissner G., Cysteine-3635 is responsible for skeletal muscle ryanodine receptor modulation by NO. PNAS. 2001; 98:11158-11162.
Aracena P., Sanchez G., Donoso P., Hamilton S. L., Hidalgo C., S-Glutathionylation Decreases Mg2+Inhibition and S-Nitrosylation Enhances Ca2+Activation of RyR1 Channels. J Biol Chem. 2003; 278:42927-42935.
Sun J., Xu L., Eu J. P., Stamler J. S., Meissner G., Nitric Oxide, NOC-12, and S-nitrosoglutathione modulate the skeletal muscle calcium release channel/ryanodine Receptor by different mechanisms. An allosteric function for O2 in S-nitrosylation of the channel. J Biol Chem. 2003; 278:8184-8189.
Aracena P., Tang W., Hamilton S., Hidalgo C., Effects of S-glutathionylation and S-nitrosylation on calmodulin binding to triads and FKBP12 binding to type 1 calcium release channels. Antioxid Redox Signal. 2005; 7:870-881.
Suhr F., Gehlert S., Grau M. and Bloch W., Skeletal Muscle Function during Exercise—Fine-Tuning of Diverse Subsystems by Nitric Oxide, Int. J. Mol. Sci. 2013, 14, 7109-7139.
Sun J., Xin C., Eu J. P., Stamler J. S., and Meissner G., Cysteine-3635 is responsible for skeletal muscle ryanodine receptor modulation by N O, PNAS, Sep. 25, 2001, vol. 98, no. 20, 11158-11162.
Quane K. A., Keating K. E., Healy J. M., Manning B. M., Krivosic-Horber R., Krivosic I., Monnier N., Lunardi J., McCarthy T. V., Mutation Screening of the RYR1 Gene in Malignant Hypertherima: Detection of a Novel Tyr to Ser Mutation in a Pedigree with Associated Central Core. Genomics., 1994; 23(1):236-9.
Chelu M. G., Goonasekera S. A., Durham W. J., Tang W., Lueck J. D., Riehl J., Pessah I. N., Zhang P., Bhattacharjee M. B., Dirksen R. T., Hamilton S. L., Heat- and Anesthesia-Induced Malignant Hyperthermia in an RyR1 Knock-In Mouse. FASEB J. 2006; 20(2):329-30.
Durham W. J., Aracena-Parks P., Long C., Rossi A. E., Goonasekera S. A., Boncompagni S., Galvan D. L., Gilman C. P., Baker M. R., Shirokova N., Protasi F., Dirksen R., Hamilton S. L., RyR1 S-Nitrosylation Underlies Environmental Heat Stroke and Sudden Death in Y522S RyR1 Knockin Mice. Cell. 2008; 133(1):53-65.
Knoblauch M., Adan Dagnino-Acosta A., Hamilton S. L., Mice with RyR1 mutation (Y524S) undergo hypermetabolic response to simvastatin, Skeletal Muscle 2013, 3:22
Lanner J. T. et al., AICAR Prevents Heat Induced Sudden Death in RyR1 Mutant Mice Independent of AMPK Activation, Nat Med.; 18(2): 244-251 (2012).
Nagaraju and Gordish, Serum Creatine Kinase analysis in mouse models of muscular dystrophy, SOP MD_M.2.2.001, TREAT NMD experimental protocols.
Rodney G G, Williams B Y, Strasburg G M, Beckingham K, Hamilton S L 2000. Regulation of RYR1 activity by Ca²⁺ and calmodulin. Biochem 39: 7807-7812.
Moore C P, Zhang J-Z, Hamilton S L 1999. A Role for Cysteine 3635 of RYR1 in Redox Modulation and Calmodulin Binding. J Biol Chem 274: 36831-36834.
Bulfield G., Siller W. G., Wight P. A., Moore K. J., X chromosome-linked muscular dystrophy (mdx) in the mouse. Proc Natl Acad Sci USA 1984; 81(4):1189-92.
Grounds M., Quantification of histopathology in Haemotoxylin and Eosin stained muscle sections, SOP DMD_M.1.2.007, TREAT NMD experimental protocols.
Barton E. R., Measuring isometric force of isolated mouse muscles in vitro, SOP DMD_M.1.2.002, TREAT NMD experimental protocols.
Novak A., et al., Modeling Catecholaminergic Polymorphic Ventricular Tachycardia using Induced Pluripotent Stem Cell-derived Cardiomyocytes, Rambam Maimonides Medical Journal, July 2012, Volume 3, Issue 3, e0015.
Hunt D. J. et al., K201 (JTV519) suppresses spontaneous Ca2+ release and [3H]ryanodine binding to RyR2 irrespective of FKBP12.6 association, Biochem J., 404, 431-438, 2007.
Itzhaki I, Maizels L, Huber I, Gepstein A, Arbel G, Caspi O, Miller L, Belhassen B, Nof E, Glikson M, Gepstein L., Modeling of catecholaminergic polymorphic ventricular tachycardia with patient-specific human-induced pluripotent stem cells, J Am Coll Cardiol. 2012 Sep. 11; 60(11):990-1000.
Pure & Appl. Chem., Vol. 45, pp. 11-30. IUPAC 1974 Recommendations, RULES FOR THE NOMENCLATURE OF ORGANIC CHEMISTRY.
Remington's Pharmaceutical Sciences, 17th Ed., Mack Publishing Company, Easton, Pa., 1985.
S. M. Berge, et al., “Pharmaceutical Salts,” J. Pharm. Sci., 1977; 66:1-19.
T. Higuchi and V. Stella, “Pro-drugs as Novel Delivery Systems,” Vol 14 of the A.C.S. Symposium Series Bioreversible Carriers in Drug Design, ed. Edward B. Roche, American Pharmaaceutical Association and Pergamon Press, 1987.
U.S. Pat. No. 4,107,288, Injectable compositions, nanoparticles useful therein, and process of manufacturing same, Richard Charles Oppenheim, Jennifer Joy Marty, Peter Speiser.
U.S. Pat. No. 5,145,684, Surface modified drug nanoparticles, Gary G. Liversidge, Kenneth C. Cundy, John F. Bishop, David A. Czekai.
WO2009063993, Fused pyridine derivative and use thereof, Tomokazu Kusumoto, Takahiro Matsumoto, Hiroyuki Nagamiya, Junya Shirai.
WO2009026444, Ryanodine channel binders and uses thereof, Elias James Corey.

### BRIEF SUMMARY OF THE INVENTION

The present invention addresses the long felt and unmet need for new and improved compounds having calcium modulatory activity and compositions thereof. The present invention also addresses the long felt and unmet need for improved compounds, and compositions thereof, that can better provide the beneficial effects of NO in myogenesis and muscle repair beyond that attainable by the use of organic nitrates. The present invention also addresses the long felt and unmet need for new and improved methods of administering calcium modulators and/or NO donors.

In stark contrast to the teachings in the art as described herein, the present invention provides novel combinations of calcium modulators and NO donators that surprisingly and unexpectedly provides beneficial activities. The surprising and unexpected beneficial activity of this combination is described in this specification in preclinical models which are predictive of human disease.

The present invention provides novel calcium modulator compounds, compositions thereof, and uses thereof. The present invention also provides novel compositions comprising calcium receptor modulators and NO donors, and uses thereof. The present invention also provides novel calcium receptor modulator compounds that have a novel dual mechanism of action including (1) calcium modulation in combination with (2) NO donor activity; compositions thereof, and uses thereof. Calcium modulation can be useful for treating muscle disease, muscle fatigue, chronic heart failure, and NO can be useful for myogenesis and muscle repair.

One aspect of the invention relates to a compound having formula I:

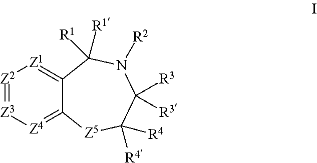

or a pharmaceutically acceptable salt, and including deuterated forms thereof, wherein: Z¹, Z², Z³, Z⁴ Z⁵, R¹, R^{1′}, R², R³, R^{3′} R⁴, and R^{4′}, are as defined throughout the specification.

Another aspect of the invention relates to a compound according to any of formulae VI(a), VI(b), VI(c), VI(d), VI(e) or VI(f):

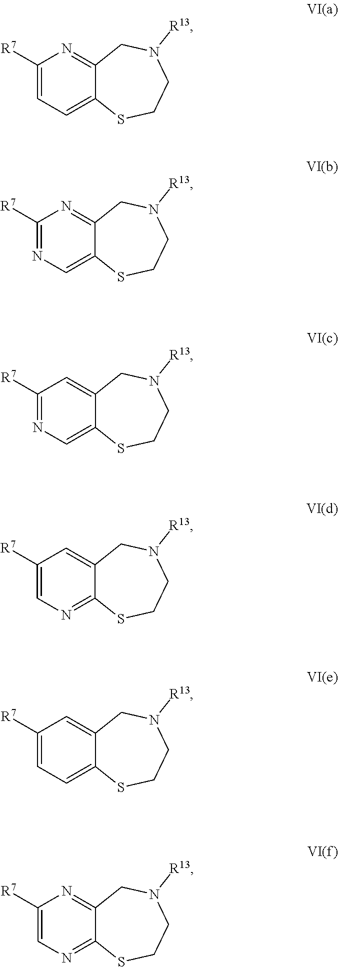

or a pharmaceutically acceptable salt, and including deuterated forms thereof, wherein: R⁷ and R¹³ are as defined in the specification.

Another aspect of the invention relates to a pharmaceutical composition comprising a compound, as described in the specification, in combination with one or more pharmaceutically acceptable excipients or carriers.

Another aspect of the invention relates to a pharmaceutical composition comprising a compound, as described in the specification, in combination with one or more NO donors and optionally one or more pharmaceutically acceptable excipients or carriers.

Another aspect of the invention relates to methods of treating or preventing conditions or diseases described in the specification by administering a compound, as described in the specification, to a patient in need of the treatment or prevention.

Another aspect of the invention relates to a compound, as described in the specification, for use in a method of treatment or prevention of a disease or condition described in the specification.

Another aspect of the invention relates to a use of a compound as described in the specification for the preparation of a medicament for use in a method of treatment or prevention of a disease or condition described in the specification.

Another aspect of the invention relates to methods of treating or preventing various muscle disorders, diseases and conditions associated with dysfunctions in calcium homeostasis or modulation, comprising administering to a subject in need of such treatment an amount of a compound or pharmaceutical composition, as described in the specification, effective to prevent or treat the disorder, disease or condition associated with a dysfunction in calcium homeostasis or modulation.

Another aspect of the invention relates to methods of treating or preventing various muscle disorders, diseases and conditions associated with dysfunctions in calcium homeostasis or modulation, comprising administering to a subject in need of such treatment an amount of a compound or pharmaceutical composition, as described in the specification, effective to prevent or treat the disorder, disease or condition associated with muscle degeneration.

Another aspect of the invention relates to methods of treating or preventing various muscle disorders, diseases and conditions associated with dysfunctions in both NO and calcium modulation, comprising administering to a subject in need of such treatment an amount of a compound or pharmaceutical composition, as described in the specification, effective to prevent or treat the disorder, disease or condition associated with dysfunctions in both NO and calcium modulation.

Another aspect of the invention relates methods of synthesizing the novel compounds described in the specification.

### BRIEF DESCRIPTION OF THE FIGURES

FIG. 1 shows the effects of the compounds of Example 2 and Example 13 on activity dependent changes in intracellular Ca²⁺ concentrations measured with a Ca²⁺ indicator (MagFluo 4) in FDB fibers from WT mice.

FIG. 2 shows the effects of the compounds of Example 2, Example 9, Example 13, Example 18 and experimental compound S107 on heating-induced intracellular calcium change in FDB fibers from YS mice.

FIG. 3 shows definitions of Transient Measurements.

CTD25: Approximates the time elapsed during the first 25% of the transient duration. The measurement assesses the time elapsed from the 75% point of upstroke to the 75% point on the downstroke relative to the peak.
Full Width Half Maximum (FWHM): The time elapsed from the 50% point of the upstroke to the 50% point of the down stroke.
CTD75: Approximates the time elapsed for 75% of the transient duration. The measurement assesses the time elapsed from the 25% point of upstroke to the 25% point on the down stroke relative to the peak.
CTD90: Approximates the time elapsed for 90% of the transient duration. The measurement assesses the time elapsed from the 10% point of upstroke to the 10% point on the down stroke relative to the peak.
Decay Time: The time elapsed from the peak to the 50% point of the down stroke T75-25: The time elapsed from the 75% point of the transient maximum to the 25% point of the transient maximum on the down stroke.
Beat Rate is assessed by measuring the number of transients observed during the recording period and extrapolating out to the number of expected beats per minute.

FIG. 4 shows the effect of the compounds of Example 2 (10 uM, 30 uM) applied to spontaneously beating cells in 4 mM Calcium Tyrode's solution, caused a reduction of proarrhythmia and calcium transient shortening.

FIG. 5 shows the effect of the compounds of Example 2 (10 uM, 30 uM) shortened CTD75 to 79% of control, with triangulation T75-25 reduced to 71% of control.

FIG. 6 shows a typical experiment demonstrating the in vitro assay developed to evaluate Ca²⁺ release in a human DMD myoblast loaded with the fluorescent Ca²⁺ indicator Fluo-4/AM.

(A) Time course of changes in background-subtracted normalized Fluo-4 fluorescence (F/F₀) following removal of Ca²⁺ from the bathing solution (top bar) and during the application of the SERCA pump inhibitor CPA in Ca²⁺-free medium (lower bar). Red dashed box: area of interest highlighted in panels B and C for analysis. (B) The rising and declining phases of the Ca²⁺ transient elicited by CPA (see panel A) were fitted by linear regression to determine the rate of Ca²⁺ release ((ΔF/F₀)/sec; red line) and extrusion ((−ΔF/F₀)/sec; green line). (C) Same plot as in panel A except that each data point was obtained by subtracting a straight line (SL) as defined by the light blue dashed line in panel A to allow for integration of the area under the curve (red) for determination of total amount of Ca²⁺ release.

FIG. 7 shows a typical experiment demonstrating the in vitro assay developed to evaluate Ca²⁺ release in a human DMD myoblast loaded with the fluorescent Ca²⁺ indicator Fluo-4/AM that includes the reintroduction of 2 mM Ca²⁺ at a later time point highlighting a further Ca²⁺ transit in the DMD myoblast via a SOCE.

FIG. 7 shows time course of changes in background-subtracted normalized Fluo-4 fluorescence (F/F₀) following removal of Ca²⁺ from the bathing solution (top bar) and during the application of the SERCA pump inhibitor CPA in Ca²⁺-free medium (lower bar), followed by reintroduction of Ca²⁺. Parameters measured are A, the peak CPA-induced calcium transit (F/F₀); B, the maximum rate of rise of CPA-induced calcium transit (+ΔF/s); C, the maximum rate of decline of CPA-induced calcium transit (−ΔF/s); D, the integrated CPA-induced calcium transit (F.s); and E, the SOCE-induced calcium transit (F/F₀).

FIG. 8 shows diaphragm muscles of untreated and Example 20 compound-treated mdx mice tested for in vitro force measurements. The compound of Example 20 demonstrated a significant improvement in both maximal force and specific force in the diaphragm of mdx mice after four weeks of daily treatment.

### DETAILED DESCRIPTION OF THE INVENTION

It should be understood that the detailed description and the specific examples while indicating various embodiments of the invention are given by way of illustration only, since various changes and modifications within the spirit and scope of the invention will become apparent to those skilled in the art from this detailed description.

As used herein and in the appended claims, the singular forms “a,” “an,” and “the” include plural references unless the content clearly dictates otherwise. All publications, patent applications, patents, and other references mentioned herein are incorporated by reference in their entirety.

### DEFINITIONS

As used herein and in the appended claims, the term “calcium modulator” refers to novel compounds of the present invention that effect the transport of calcium through cellular membranes.

Terms used herein may be preceded and/or followed by a single dash, “−”, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” unless a dash indicates otherwise. For example, alkoxycarbonyloxy and —OC(O)Oalkyl indicate the same point of attachment to the parent moiety.

By alkyl is meant herein a saturated hydrocarbyl radical, which may be straight-chain, cyclic or branched (typically straight-chain unless the context dictates to the contrary). Where an alkyl group has one or more sites of unsaturation, these may be constituted by carbon-carbon double bonds or carbon-carbon triple bonds. Where an alkyl group comprises a carbon-carbon double bond this provides an alkenyl group; the presence of a carbon-carbon triple bond provides an alkynyl group. In one example, alkyl, alkenyl and alkynyl groups will comprise from 1 to 25 carbon atoms. In another example, alkyl, alkenyl and alkynyl groups will comprise from 1 to 10 carbon atoms. In another example, alkyl, alkenyl and alkynyl groups will comprise from 1 to 6 carbon atoms. In another example, alkyl, alkenyl and alkynyl groups will comprise from 1 to 4 carbon atoms. In another example, alkyl, alkenyl and alkynyl groups will comprise from 1 to 3 carbon atoms. In another example, alkyl, alkenyl and alkynyl groups will comprise from 1 to 2 carbon atoms. In another example, alkyl groups will comprise 1 carbon atom. It is understood that the lower limit in alkenyl and alkynyl groups is 2 carbon atoms and in cycloalkyl groups 3 carbon atoms.

Alkyl, alkenyl or alkynyl groups may be substituted, for example once, twice, or three times, e.g. once, i.e. formally replacing one or more hydrogen atoms of the alkyl group. Examples of such substituents are halo (e.g. fluoro, chloro, bromo and iodo), aryl, hydroxy, nitro, amino, alkoxy, alkylthio, carboxy, cyano, thio, formyl, ester, acyl, thioacyl, amido, sulfonamido, carbamate and the like.

Alkyl, alkenyl or alkynyl groups include monovalent and bivalent groups, such as an alkenylene group.

Halo or halogen is fluoro, bromo, chloro or iodo.

By acyl and thioacyl are meant the functional groups of formulae —C(O)-alkyl or —C(S)-alkyl respectively, where alkyl is as defined hereinbefore.

By ester is meant a functional group comprising the moiety —OC(═O)—.

By carbamate is meant a functional group comprising the moiety —N(H)C(═O)O—, in which each hydrogen atom depicted may be replaced with alkyl or aryl.

Alkyloxy (synonymous with alkoxy) and alkylthio moieties are of the formulae —O-alkyl and —S-alkyl respectively, where alkyl is as defined hereinbefore.

Deuterated alkyl is meant herein as an alkyl group as defined herein, wherein one or more hydrogen atoms of the alkyl group is replaced with deuterium. When more than one deuterated alkyl group exists in a molecule disclosed herein, each deuterated C₁-C₆alky group can be the same or different.

Deuterated —(C₁-C₆)alkyl is meant herein as a —(C₁-C₆)alkyl group wherein one or more hydrogen atoms of the —(C₁-C₆)alkyl group is replaced with deuterium. When more than one deuterated —(C₁-C₆)alkyl group exists in a molecule disclosed herein, each deuterated C₁-C₆alkyl group can be the same or different.

Deuterated alkoxy is meant herein as an —O-alkyl group, wherein one or more hydrogen atoms of the alkyl group is replaced with deuterium. When more than one deuterated alkyl group exists in a molecule disclosed herein, each deuterated —(C₁-C₆)alkyl group can be the same or different.

Deuterated —(C₁-C₆)alkoxy is meant herein as —O—(C₁-C₆)alkyl group wherein one or more hydrogen atoms of the —(C₁-C₆)alkyl group is replaced with deuterium. When more than one deuterated —(C₁-C₆)alkyl group exists in a molecule disclosed herein, each deuterated C₁-C₆alkyl group can be the same or different.

Deuterated methoxy is meant herein as —OCD₁₋₃. It is to be understood that —OCD₁₋₃ is meant to include either —OCH₂D, —OCHD₂, or —OCD₃. When more than one deuterated methoxy group exists in a molecule disclosed herein, each deuterated methoxy group can be the same or different.

By amino group is meant herein a group of the formula —N(R)₂ in which each R is independently hydrogen, alkyl or aryl. For example, R can be an unsaturated, unsubstituted C₁₋₆ alkyl such as methyl or ethyl. In another example, the two R groups attached to the nitrogen atom N are connected to form a ring. One example where the two Rs attached to nitrogen atom N are connected is whereby —R—R— forms an alkylene diradical, derived formally from an alkane from which two hydrogen atoms have been abstracted, typically from terminal carbon atoms, whereby to form a ring together with the nitrogen atom of the amine. As is known the diradical in cyclic amines need not necessarily be alkylene: morpholine (in which —R—R— is —(CH₂)₂O(CH₂)₂—) is one such example from which a cyclic amino substituent may be prepared.

An NO donor is a group that can generate or release free NO under physiological or non-physiological conditions. Such conditions include, but are not limited to, when the NO donor is hydrolysed or metabolized, for example, by a CYP450 enzyme. Typical NO donors include organic nitrates (i.e., RONO₂ wherein R is an optionally substituted alkyl group), diazeniumdiolates (NOVOates), furoxanes or syndonimines.

References to amino herein are also to be understood as embracing within their ambit quaternised or protonated derivatives of the amines resultant from compounds comprising such amino groups. Examples of the latter may be understood to be salts such as hydrochloride salts.

Calcium homeostasis is meant herein as the regulation of the concentration of calcium ions in intracellular and extracellular fluid.

Calcium ion channel modulators is meant herein as a substance that changes or regulates the activity of calcium ion channels.

“Aryl” means a monovalent, monocyclic, or polycyclic radical having 6 to 14 ring carbon atoms. The monocyclic aryl radical is aromatic and whereas the polycyclic aryl radical may be partially saturated, at least one of the rings comprising a polycyclic radical is aromatic. The polycyclic aryl radical includes fused, bridged, and spiro ring systems. Any 1 or 2 ring carbon atoms of any nonaromatic rings comprising a polycyclic aryl radical may be replaced by a —C(O)—, —C(S)—, or —C(═NH)— group. Unless stated otherwise, the valency may be located on any atom of any ring of the aryl group, valency rules permitting. Representative examples include phenyl, naphthyl, indanyl, and the like.

“Carbonyl” means a —C(O)— group.

“Cycloalkyl” means a monocyclic or polycyclic hydrocarbon radical having 3 to 13 carbon ring atoms. The cycloalkyl radical may be saturated or partially unsaturated, but cannot contain an aromatic ring. The cycloalkyl radical includes fused, bridged and spiro ring systems. Examples of such radicals include cyclopropyl, cyclobutyl, cyclopentyl and cyclohexyl.

“Heteroaryl” means a monovalent monocyclic or polycyclic radical having 5 to 14 ring atoms of which one or more of the ring atoms, for example one, two, three, or four ring atoms, are heteroatoms independently selected from —O—, —S(O)ₙ— (n is 0, 1, or 2), —N—, —N(Rₓ), and the remaining ring atoms are carbon atoms, where Rₓ is hydrogen, alkyl, hydroxy, alkoxy, —C(O)R⁰ or —S(O)₂R⁰, where R⁰ is alkyl. The monocyclic heteroaryl radical is aromatic and whereas the polycyclic heteroaryl radical may be partially saturated, at least one of the rings comprising a polycyclic radical is aromatic. The polycyclic heteoaryl radical includes fused, bridged and spiro ring systems. Any 1 or 2 ring carbon atoms of any nonaromatic rings comprising a polycyclic heteroaryl radical may be replaced by a —C(O)—, —C(S)—, or —C(═NH)— group. Unless stated otherwise, the valency may be located on any atom of any ring of the heteroaryl group, valency rules permitting. In particular, when the point of valency is located on the nitrogen, then R^{x} is absent. More specifically, the term heteroaryl includes, but is not limited to, 1,2,4-triazolyl, 1,3,5-triazolyl, phthalimidyl, pyridinyl, pyrrolyl, imidazolyl, thienyl, furanyl, indolyl, 2,3-dihydro-1H-indolyl (including, for example, 2,3-dihydro-1H-indol-2-yl, 2,3-dihydro-1H-indol-5-yl, and the like), isoindolyl, indolinyl, isoindolinyl, benzimidazolyl, benzodioxol-4-yl, benzofuranyl, cinnolinyl, indolizinyl, naphthyridin-3-yl, phthalazin-3-yl, phthalazin-4-yl, pteridinyl, purinyl, quinazolinyl, quinoxalinyl, tetrazoyl, pyrazolyl, pyrazinyl, pyrimidinyl, pyridazinyl, oxazolyl, isooxazolyl, oxadiazolyl, benzoxazolyl, quinolinyl, isoquinolinyl, tetrahydroisoquinolinyl (including, for example, tetrahydroisoquinolin-4-yl, tetrahydroisoquinolin-6-yl, and the like), 2,3,3a,7a-tetrahydro-1H-isoindolyl, pyrrolo[3,2-c]pyridinyl (including, for example, pyrrolo[3,2-c]pyridin-2-yl, pyrrolo[3,2-c]pyridin-7-yl, and the like), benzopyranyl, thiazolyl, isothiazolyl, thiadiazolyl, benzothiazolyl, benzothienyl, and the N-oxide derivatives thereof.

“Heterocyclyl” means a monovalent, monocyclic or polycyclic hydrocarbon radical having 3 to 13 ring atoms of which one or more of the ring atoms, for example 1, 2, 3 or 4 ring atoms, are heteroatoms independently selected from —O—, —S(O)ₙ— (n is 0, 1, or 2), —N═ and —N(R^{y})— (where R^{y} is hydrogen, alkyl, hydroxy, alkoxy, —C(O)R⁰ or —S(O)₂R⁰, where R⁰ is alkyl, as defined herein), and the remaining ring atoms are carbon. The heterocycloalkyl radical may be saturated or partially unsaturated, but cannot contain an aromatic ring. The heteocycloalkyl radical includes fused, bridged and spiro ring systems. Any 1 or 2 ring carbon atoms independently may be replaced by a —C(O)—, —C(S)—, or —C(═NH)— group. Unless otherwise stated, the valency of the group may be located on any atom of any ring within the radical, valency rules permitting. In particular, when the point of valency is located on a nitrogen atom, R^{y} is absent. More specifically the term heterocycloalkyl includes, but is not limited to, azetidinyl, pyrrolidinyl, 2-oxopyrrolidinyl, 2,5-dihydro-1H-pyrrolyl, piperidinyl, 4-piperidonyl, morpholinyl, piperazinyl, 2-oxopiperazinyl, tetrahydropyranyl, 2-oxopiperidinyl, thiomorpholinyl, thiamorpholinyl, perhydroazepinyl, pyrazolidinyl, imidazolinyl, imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydroindolyl, octahydroisoindolyl, decahydroisoquinolyl, tetrahydrofuryl, 1,4-dioxa-8-azaspiro[4.5]decan-8-yl and tetrahydropyranyl, and the N-oxide derivatives thereof

“Heterocyclylalkyl” means a heterocyclyl group appended to a parent moiety via an alkyl group, as defined herein.

“Spiro ring” refers to a ring originating from a particular annular carbon of another ring.

“Patient” and “subject” for the purposes of the present invention includes humans and other animals, particularly mammals, and other organisms. Thus the methods are applicable to both human therapy and veterinary applications. In another embodiment the patient is a mammal, and in another embodiment the patient is human.

All of the compounds disclosed herein can exist as single stereoisomers (including single enantiomers and single diastereomers), racemates, mixtures of enantiomers and diastereomers and polymorphs. Stereoisomers of the compounds in this disclosure include geometric isomers and optical isomers, such as atropisomers. The compounds disclosed herein can also exist as geometric isomers. All such single stereoisomers, racemates and mixtures thereof, and geometric isomers are intended to be within the scope of the compounds disclosed herein. Compounds of the present invention may exist in their tautomeric form. All such tautomeric forms are contemplated herein as part of the present invention.

Individual stereoisomers of the compounds of the invention may, for example, be substantially free of other isomers (e.g., as a pure or substantially pure optical isomer having a specified activity), or may be admixed, for example, as racemates, or as mixtures enriched by one stereoisomer. The chiral centers of the present invention may have the S or R configuration as defined by the IUPAC 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid or base, followed by crystallization.

When chemical structures are depicted or described, unless explicitly stated otherwise, all carbons are assumed to have hydrogen substitution to conform to a valence of four. Sometimes a particular atom in a structure is described in textual Formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

It is assumed that when considering generic descriptions of compounds of the disclosed herein for the purpose of constructing a compound, such construction results in the creation of a stable structure. That is, one of ordinary skill in the art would recognize that theoretically some constructs which would not normally be considered as stable compounds (that is, sterically practical and/or synthetically feasible).

The compounds described herein, as well as their pharmaceutically acceptable salts or other derivatives thereof, can optionally exist in isotopically-labeled form, in which one or more atoms of the compounds are replaced by an atom having the same atomic number but an atomic mass different from the atomic mass usually found in nature. Examples of isotopes that can be incorporated into compounds described herein include isotopes of hydrogen, carbon, nitrogen, oxygen, phosphorous, sulfur, fluorine and chloride, such as ²H (deuterium), ³H (tritium), ¹³C, ¹⁴C, ¹⁵N, ¹⁸0, ¹⁷0, ³¹P, ³²P, ³⁵S, ¹⁸F and ³⁶Cl, respectively. Isotopically labeled compounds described herein, as well as pharmaceutically acceptable salts, esters, SMDCs, solvates, hydrates or other derivatives thereof, generally can be prepared by carrying out the procedures disclosed in the Schemes and/or in the Examples below, by substituting a readily available isotopically labeled reagent for a non-isotopically labeled reagent. When a particular hydrogen position is replaced with a “D” or “deuterium”, it is to be understood that the abundance of deuterium at that position is substantially greater than the natural abundance of deuterium, which is 0.015%, and typically has at least 50% deuterium incorporation at that position. In one embodiment, one or more hydrogens attached to one or more sp³ carbons in the compounds disclosed herein are replaced with deuterium. In another embodiment, one or more hydrogens attached to one or more sp² carbons in the compounds disclosed herein are replaced with deuterium.

“Optional” or “optionally” means that the subsequently described event or circumstance can or cannot occur, and that the description includes instances where said event or circumstance occurs and instances in which it does not. One of ordinary skill in the art would understand that with respect to any molecule described as containing one or more optional substituents, only sterically practical and/or synthetically feasible compounds are meant to be included. “Optionally substituted” means substituted or unsubstituted and refers to all subsequent modifiers in a term unless otherwise specified. So, for example, in the term “optionally substituted arylalkyl,” both the “alkyl” portion and the “aryl” portion of the molecule can be substituted or unsubstituted.

Unless otherwise specified, the term “optionally substituted” applies to the chemical moiety immediately following. For instance, if a variable group (such as R) is defined as aryl, optionally substituted alkyl, or cycloalkyl, then only the alkyl group is optionally substituted.

A “pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17th Ed., Mack Publishing Company, Easton, Pa., 1985, which is incorporated herein by reference or S. M. Berge, et al., “Pharmaceutical Salts,” J. Pharm. Sci., 1977; 66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts and the like. Preferable salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, tromethamine, N-methylglucamine, polyamine resins, and the like. Exemplary organic bases are isopropylamine, diethylamine, ethanolamine, trimethylamine, dicyclohexylamine, choline, and caffeine.

All of the compounds disclosed herein include either their free base form or their pharmaceutically acceptable salts whether it is stated in the specification that these compounds can exist as their pharmaceutically acceptable salt or not.

Prodrugs of the compounds disclosed herein are also contemplated as part of the invention.

“Prodrug” refers to compounds that are transformed (typically rapidly) in vivo to yield the parent compound of the above formulae, for example, by hydrolysis in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides and secondary and tertiary alkyl amides (for example with between about one and about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Therapeutically effective amount” is an amount of a compound of the invention, that when administered to a patient, effectively treats the disease. The amount of a compound of the invention which constitutes a “therapeutically effective amount” will vary depending upon a sundry of factors including the activity, metabolic stability, rate of excretion and duration of action of the compound, the age, weight, general health, sex, diet and species of the patient, the mode and time of administration of the compound, the concurrent administration of adjuvants or additional therapies and the severity of the disease for which the therapeutic effect is sought. The therapeutically effective amount for a given circumstance can be determined without undue experimentation.

“Treating” or “treatment” of a disease, disorder, or syndrome, as used herein, includes (i) preventing the disease, disorder, or syndrome from occurring in a human, i.e., causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As is known in the art, adjustments for systemic versus localized delivery, the age, weight, general health, sex, diet and species of the patient, the mode and time of administration of the compound, the concurrent administration of adjuvants or additional therapeutically active ingredients and the severity of the disease for which the therapeutic effect is sought may be necessary, and will be ascertainable with routine experimentation.

In addition, the compounds of this disclosure can exist in unsolvated as well as solvated forms with pharmaceutically acceptable solvents such as water, ethanol, and the like. In general, the solvated forms are considered equivalent to the unsolvated forms for the purposes of the compounds of this disclosure.

### Pharmaceutical Formulations and Dosage Forms

Administration of the compounds of this disclosure, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, preferably in unit dosage forms suitable for simple administration of precise dosages.

The compositions will include a conventional pharmaceutical carrier, excipient, and/or diluent and a compound of this disclosure as the/an active agent, and, in addition, can include carriers and adjuvants, etc.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It can also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the compounds in this disclosure can also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Compositions suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One preferable route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms can also comprise buffering agents.

Solid dosage forms, as described above, can be prepared with coatings and shells, such as enteric coatings and others well known in the art. They can contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of this disclosure, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, can contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of this disclosure with, for example, suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this disclosure include ointments, powders, sprays, and inhalants. The active component is admixed under sterile conditions with a physiologically acceptable carrier and any preservatives, buffers, or propellants as can be required. Ophthalmic formulations, eye ointments, powders, and solutions are also contemplated for the compounds in this disclosure.

Compressed gases can be used to disperse a compound of this disclosure in aerosol form. Inert gases suitable for this purpose are nitrogen, carbon dioxide, etc.

Generally, depending on the intended mode of administration, the pharmaceutically acceptable compositions will contain about 1% to about 99% by weight of a compound(s) of this disclosure, or a pharmaceutically acceptable salt thereof, and 99% to 1% by weight of a suitable pharmaceutical excipient. In one example, the composition will be between about 5% and about 75% by weight of a compound(s) of this disclosure, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of this disclosure, or a pharmaceutically acceptable salt thereof, for treatment of a disease-state in accordance with the teachings of this disclosure.

The compounds of this disclosure, or their pharmaceutically acceptable salts, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of this disclosure can be administered to a patient at dosage levels in the range of about 0.1 to about 5,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

The compositions will include a conventional pharmaceutical carrier or excipient and a compound of this disclosure as the/an active agent, and, in addition, can include other medicinal agents and pharmaceutical agents. Compositions of the compounds in this disclosure can be used in combination with anticancer and/or other agents that are generally administered to a patient being treated for cancer, e.g. surgery, radiation and/or chemotherapeutic agent(s). Chemotherapeutic agents that can be useful for administration in combination with compounds of Formula I in treating cancer include alkylating agents, platinum containing agents.

If formulated as a fixed dose, such combination products employ the compounds of this disclosure within the dosage range described above and the other pharmaceutically active agent(s) within its approved dosage range. Compounds of this disclosure can alternatively be used sequentially with known pharmaceutically acceptable agent(s) when a combination formulation is inappropriate.

### Aspects and Embodiments of the Invention

The following Aspects and Embodiments are intended to represent non-limiting examples of various aspects and embodiments of the invention. These embodiments illustrative in nature and are not intended to exclude other embodiments or limit the scope of the invention. Thus, a definition of a particular substituent is not intended to exclude other embodiments of that substituent unless specifically indicated.

One aspect of the invention relates to a compound having formula I:

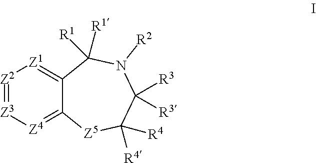

or a pharmaceutically acceptable salt, and including deuterated forms thereof, wherein:

Z¹ is —C(R⁸)— or —N—;

Z² is —C(R⁷)— or —N—;

Z³ is —C(R⁶)— or —N—;

Z⁴ is —C(R⁵)— or —N—;

Z⁵ is —O—, —S—, —S(O)—, —S(O)₂—, —NR^{x}— or —C(R^{x})₂—;

R¹, R^{1′}, R³, and R^{3′} are each independently selected from D, R^{x}, C(H)₂OR^{x}, C(H)₂OC(═O)R^{x}, C(═O)OR^{x}, C(═O)N(H)R^{x}, C(═O)R^{x}, and OC(═O)R^{x}; and optionally R¹ and R^{1′} taken together form oxo (═O); and optionally R³ and R^{3′} taken together form oxo (═O);

each of R⁵, R⁶, R⁷ and R⁸, which can be the same or different, are independently selected from H, D, halo, R^{x}, —OR^{x}, —SR^{x}, —N(R^{x})₂, —N(R^{x})C(═O)OR^{x}, —C(═O)N(R^{x})₂, —C(═O)OR^{x}, —C(═O)R^{x}, —OC(═O)R^{x}, —NO₂, —CN, —N₃, and —P(═O)(R^{x})₂; or

R⁵ and R⁶, together with the carbon atoms to which they are respectively attached, form an unsubstituted or substituted cycloalkyl or heterocyclic ring, wherein the substituents are one to three substituents independently selected from halo, aryl, R^{x}, hydroxyl nitro, amino, alkoxy, alkylthio, —CO₂H, and CN; or

R⁶ and R⁷, together with the carbon atoms to which they are respectively attached, form an unsubstituted or substituted cycloalkyl or heterocyclic ring, wherein the substituents are one to three substituents independently selected from halo, aryl, R^{x}, hydroxyl nitro, amino, alkoxy, alkylthio, —CO₂H, and CN;

R² is -L¹-L²-G;

L¹ is —C(O)—, —C(O)C(O)— or —(C₁-C₆)alkyl optionally substituted with one to three groups selected from halo; —(C₁-C₃)alkyl optionally substituted with 1-3 groups selected from halo and D; —(C₁-C₃)alkoxy optionally substituted with 1-3 groups independently selected from halo and D; or a spiro-(C₃-C₆)cycloalkyl optionally substituted with 1-2 groups selected from halo, D, methyl, and halogenated methyl;

L² is —O—, oxycarbonylaryl or oxycarbonylheteroaryl, wherein each aryl or heteroaryl group of L² is optionally substituted with one to three substituents independently selected from halo, D, —(C₁-C₆)alkyl, hydroxyl, nitro, amino, alkoxy, alkylthio, —CO₂H, and CN;

G is either absent or is one to three NO donors, provided that when G is absent, at least one of Z¹, Z², Z³ or Z⁴ is a nitrogen atom;

R⁴ and R^{4′} are each independently selected from H, D, and R^{x}, or are combined to form oxo; or

R³ and R⁴ together with the carbon atoms to which they are respectively attached, form an unsubstituted or substituted cycloalkyl or heterocyclic ring, wherein the substituents are one to three substituents independently selected from halo, aryl, R^{x}, hydroxyl nitro, amino, alkoxy, alkylthio, —CO₂H, and CN;

each R^{x} is independently selected from H, D, alkyl, alkenyl, alkynyl, alkoxy, alkoxyalkyl, cycloalkyl, heterocyclyl, aryl, heteroaryl, cycloalkylalkyl, heterocyclylalkyl, alkylaryl, and heteroaryl, alkyl, wherein the alkyl, alkenyl or alkynyl portions of R^{x} can be optionally substituted with one to three substituents selected from D, halo, hydroxyl, nitro, amino, —CO₂H, and CN.

In another embodiment of formula I, Z⁵ is —O—, —S—, —NR^{x}— or —C(R^{x})₂—.

When G is absent in any of the embodiments described herein, it is understood that hydrogen is at the position where G would have been attached.

In selected embodiments for the compounds of formula I, the compounds will have a molecular weight of less than 700, in other selected embodiments, less than 600. In still other embodiments, the compounds of formula I will have a molecular weight of from about 300 to 550.

In yet another group of selected embodiments, the compounds described herein will preferably have an octanol/water partition coefficient (log P) of less than 7. A variety of methods are known in the art for the calculation of a compound's log P (termed clogP).

In still another group of selected embodiments, the compounds described herein will have one or two NO donor groups, generally a single NO donor group.

Another embodiment of the compound of formula I relates to a compound having formula II:

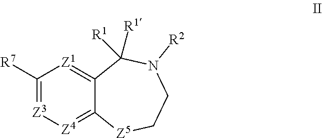

or a pharmaceutically acceptable salt, and including deuterated forms thereof, wherein:

Z¹ is —C(R⁸)— or —N—;

Z³ is —C(R⁶)— or —N—;

Z⁴ is —C(R⁵)— or —N—;

Z⁵ is —O—, —S—, —S(O)—, —S(O)₂—;

R¹ and R^{1′} are each independently selected from D and H;

each of R⁵, R⁶, and R⁸, which can be the same or different, are independently selected from H, D, halo, R^{x}, —OR^{x}, —SR^{x}, —N(R^{x})₂, —N(R^{x})C(═O)OR^{x}, —C(═O)N(R^{x})₂, —C(═O)OR^{x}, —C(═O)R^{x}, —OC(═O)R^{x}, —NO₂, —CN, —N₃, and —P(═O)(R^{x})₂; or

R⁵ and R⁶, together with the carbon atoms to which they are respectively attached, form an unsubstituted or substituted cycloalkyl or heterocyclic ring, wherein the substituents are one to three substituents independently selected from halo, aryl, R^{x}, hydroxyl, nitro, amino, alkoxy, alkylthio, —CO₂H, and CN;

R² is -L¹-L²-G;

L¹ is —C(O)—, —C(O)C(O)—, —(C₁-C₆)alkyl optionally substituted with one or more groups selected from halo; —(C₁-C₃)alkyl optionally substituted with 1-3 groups selected from halo and D; —(C₁-C₃)alkoxy optionally substituted with 1-3 groups independently selected from halo and D; or a spiro-(C₃-C₆)cycloalklyl optionally substituted with 1-2 groups selected from halo, D, methyl, and halogenated methyl;

L² is —O—, oxycarbonylaryl or oxycarbonylheteroaryl, wherein each aryl or heteroaryl group of L² is optionally substituted with one to three substituents independently selected from halo, D, aryl, —(C₁-C₆)alkyl, hydroxyl, nitro, amino, alkoxy, alkylthio, —CO₂H, and CN;

R⁷ is selected from halo, D, R^{x}, —OR^{x}, —SR^{x}, —S(O)R^{x}, —S(O)₂R^{x}, —N(R^{x})₂, —N(R^{x})C(═O)OR^{x}, —C(═O)N(R^{x})₂, —C(═O)OR^{x}, —C(═O)R^{x}, —OC(═O)R^{x}, —NO₂, —CN, —N₃, and —P(═O)(R^{x})₂;

G is absent or an NO donor, provided that when G is absent, at least one of Z¹, Z³ or Z⁴ is a nitrogen atom; and

each R^{x} is independently selected from H, D, alkyl, alkenyl, alkynyl, alkoxy, alkoxyalkyl, cycloalkyl, heterocyclyl, aryl, heteroaryl, cycloalkylalkyl, heterocyclylalkyl, aryl, alkyl, and heteroaryl, alkyl, wherein the alkyl, alkenyl or alkynyl portions of R^{x} can be optionally substituted with one to three substituents selected from D, halo, hydroxyl, nitro, amino, alkoxy, alkylthio, —CO₂H, and CN.

In another embodiment of formula II, wherein Z⁵ is —O— or —S—.

In other embodiments of formula I and II, including the embodiments of formula I and II described in this specification, Z¹ is —N—; Z³ is —(CH)— and Z⁴ is —(CH)—.

In other embodiments of formula I and II, including the embodiments of formula I and II described in this specification, Z¹ is —N—; Z³ is —N— and Z⁴ is —(CH)—.

In other embodiments of formula I and II, including the embodiments of formula I and II described in this specification, Z¹ is —N—; Z³ is —(CH)— and Z⁴ is —N—.

In other embodiments of formula I and II, including the embodiments of formula I and II described in this specification, including the embodiments of formula I and II described in this specification, Z¹ is —(CH)—; Z³ is —N— and Z⁴ is —(CH)—.

In other embodiments of formula I and II, including the embodiments of formula I and II described in this specification, Z¹ is —(CH)—; Z³ is —N— and Z⁴ is —N—.

In other embodiments of formula I and II, including the embodiments of formula I and II described in this specification, Z¹ is —(CH)—; Z³ is —(CH)— and Z⁴ is —N—.

In other embodiments of formula I and II, including the embodiments of formula I and II described in this specification, Z¹ is —N—; Z³ is —C(R⁶)— and Z⁴ is —C(R⁵)—.

In other embodiments of formula I and II, including the embodiments of formula I and II described in this specification, Z¹ is —N—; Z³ is —N— and Z⁴ is —N—.

In other embodiments of formula I and II, including the embodiments of formula I and II described in this specification, R¹, R^{1′}, R³, and R^{3′} are each H.

In other embodiments of formula I and II, including the embodiments of formula I and II described in this specification, R¹ and R^{1′} are each D; and R³ and R^{3′} are each H.

In other embodiments of formula I and II, including the embodiments of formula I and II described in this specification, L¹ is —C(O)C(O)—, and L² is —O—.

In other embodiments of formula I and II, including the embodiments of formula I and II described in this specification, L¹ is —C(O)—, and L² is —O—.

In other embodiments of formula I and II, including the embodiments of formula I and II described in this specification, L¹ is —(C₁-C₆)alkyl optionally substituted with one to three groups selected from halo, and D; —(C₁-C₃)alkyl optionally substituted with 1-3 groups selected from halo and D; —(C₁-C₃)alkoxy optionally substituted with 1-3 groups independently selected from halo and D; or a spiro-(C₃-C₆)cycloalklyl optionally substituted with 1-2 groups selected from halo, D, methyl, and halogenated methyl; and L² is oxycarbonylaryl or oxycarbonylheteroaryl, wherein each aryl or heteroaryl group of L² is optionally substituted with one to three substituents independently selected from halo, —(C₁-C₆)alkyl, hydroxyl, nitro, amino, alkoxy, alkylthio, —CO₂H, and CN.

In other embodiments of the compounds having formula I and II, including the embodiments of formula I and II described in this specification, Z⁵ is S. In other embodiments of the compounds having formula I and II, including the embodiments of formula I and II described in this specification, Z⁵ is O. In other embodiments of the compounds having formula I and II, including the embodiments of formula I and II described in this specification, Z⁵ is —S(O)—. In other embodiments of the compounds having formula I and II, including the embodiments of formula I and II described in this specification, Z⁵ is —S(O)₂—.

In other embodiments of the compounds having formula I and II, including the embodiments of formula I and II described in this specification, R⁷ is selected —OR^{x}, wherein R^{x} is as defined in the specification.

In other embodiments of the compounds having formula I and II, including the embodiments of formula I and II described in this specification, the compound is present in the form of a pharmaceutically acceptable salt, and/or a deuterated form thereof

In other embodiments of the compounds having formulae I or II, G is absent and Z¹ is N.

In other embodiments of the compounds having formulae I or II, G is absent; Z¹ is N; and R¹ and R^{1′} are each D.

In other embodiments of the compounds having formulae I or II, G is absent, and Z³ and Z⁴ are each N.

In other embodiments of the compounds having formulae I or II, G is absent, Z³ and Z⁴ are each N; and R¹ and R^{1′} are each D.

In other embodiments of the compounds having formulae I or II, G is an NO donor selected from organic nitrates (i.e., RONO₂ wherein R is an optionally substituted alkyl group), diazeniumdiolates (NOVOates), furoxanes or syndonimines.

In other embodiments of the compounds having formula I and II, including the embodiments of formula I and II described in this specification, or a pharmaceutically acceptable salt, and including deuterated forms thereof.

In another embodiment, G is absent or an NO donor selected from —(C₁-C₁₀)alkyl substituted with 1 or 2 —ONO₂ groups, —C(H)₂—O—R⁹, —(C₁-C₆)alkylene-O—C(H)₂C(H)(ONO₂)—(C₁-C₆)alkyl, -phenylene-R⁹, —(C₁-C₆)alkylene-S(O)₂N(H)(OH),

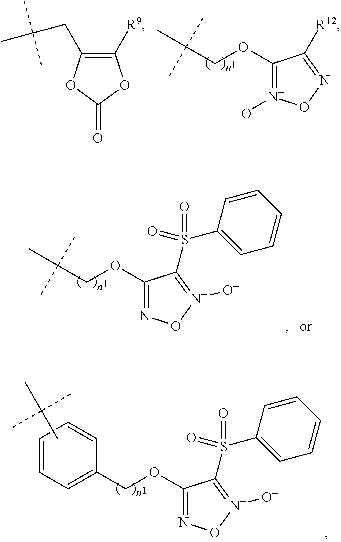

wherein each alkylene group of G is optionally substituted with one or more substituents selected from halo, aryl, hydroxyl, nitro, amino, alkoxy, alkylthio, —CO₂H, and CN;

R⁹ is —(C₂-C₁₀)alkyl substituted with 1 or 2 —ONO₂ groups;

R¹² is H or —(C₁-C₃)alkyl and

n¹ is an integer from 2-5.

Another embodiment of the compound of formulae I and II, relate to a compound of formula III:

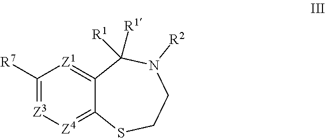

or a pharmaceutically acceptable salt, and including deuterated forms thereof, wherein:

Z¹ is —C(R⁸)— or —N—;

Z³ is —C(R⁶)— or —N—;

Z⁴ is —C(R⁵)— or —N—;

R¹ and R^{1′} are each independently selected from D or H;

each of R⁵, R⁶, and R⁸, which can be the same or different, are independently selected from H, D, halo, —(C₁-C₆)alkyl optionally substituted with halo, —O—(C₁-C₆)alkyl optionally substituted with halo, SR^{x}, N(R^{x})₂, N(R^{x})C(═O)OR^{x}, C(═O)N(R^{x})₂, C(═O)OR^{x}, C(═O)R^{x}, OC(═O)R^{x}, NO₂, —CN, and —N₃; or

R⁵ and R⁶, together with the carbon atoms to which they are respectively attached, form an unsubstituted or substituted cycloalkyl or heterocyclic ring, wherein the substituents are one to three substituents independently selected from halo, R^{x}, hydroxyl, nitro, amino, alkoxy, alkylthio, —CO₂H, and CN;

R² is -L¹-L²-G;

L¹ is —C(O)—, —C(O)C(O)— or —(C₁-C₆)alkyl optionally substituted with 1-3 groups selected from halo and D; —(C₁-C₃)alkoxy optionally substituted with 1-3 groups independently selected halo and D; or a spiro-(C₃-C₆)cycloalklyl optionally substituted with 1-2 groups selected from halo, D, methyl, and halogenated methyl;

L² is —O—, oxycarbonylaryl or oxycarbonylheteroaryl, wherein each aryl or heteroaryl group of L² is optionally substituted with one to three substituents independently selected from halo, D, —(C₁-C₆)alkyl, hydroxyl, nitro, amino, alkoxy, alkylthio, —CO₂H, and CN;

R⁷ is selected from halo, D, R^{x}, —OR^{x}, —SR^{x}, —N(R^{x})₂, —N(R^{x})C(═O)OR^{x}, —C(═O)N(R^{x})₂, —C(═O)OR^{x}, —C(═O)R^{x}, —OC(═O)R^{x}, —NO₂, —CN, —N₃, and —P(═O)(R^{x})₂;

G is absent or an NO donor selected from —(C₁-C₁₀)alkyl substituted with 1 or 2 —ONO₂ groups, —C(H)₂—O—R⁹, —(C₁-C₆)alkylene-O—C(H)₂C(H)(ONO₂)—(C₁-C₆)alkyl, -phenylene-R⁹, —(C₁-C₆)alkylene-S(O)₂N(H)(OH),

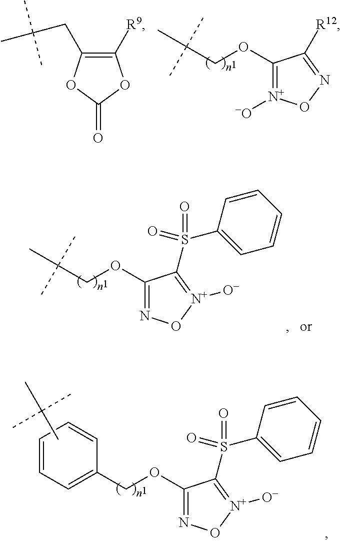

wherein each alkylene group of G is optionally substituted with one to three substituents independently selected from halo, aryl, hydroxyl, nitro, amino, alkoxy, alkylthio, —CO₂H, and CN, provided that when G is absent, at least one of Z¹, Z³ or Z⁴ is a nitrogen atom;

R⁹ is —(C₂-C₁₀)alkyl substituted with 1 or 2 —ONO₂ groups;

R¹² is H or —(C₁-C₃)alkyl;

n¹ is an integer from 0-5; and

each R^{x} is independently selected from H, D, alkyl, alkenyl, alkynyl, alkoxy, alkoxyalkyl, cycloalkyl, heterocyclyl, aryl, heteroaryl, cycloalkylalkyl, heterocyclylalkyl, and alkyl, and heteroaryl, alkyl, wherein the alkyl, alkenyl or alkynyl portion of R^{x} can be optionally substituted with one or more substituents independently selected from halo, D, aryl, hydroxyl, nitro, amino, alkoxy, alkylthio, —CO₂H, and CN.

In other embodiments of formula III, Z¹ is —N—; Z³ is —(CH)— and Z⁴ is —(CH)—.

In other embodiments of formula I and II, Z¹ is —N—; Z³ is —N— and Z⁴ is —(CH)—.

In other embodiments of formula III, Z¹ is —N—; Z³ is —(CH)— and Z⁴ is —N—.

In other embodiments of formula III, Z¹ is —(CH)—; Z³ is —N— and Z⁴ is —(CH)—.

In other embodiments of formula III, Z¹ is —(CH)—; Z³ is —N— and Z⁴ is —N—.

In other embodiments of formula III, Z¹ is —(CH)—; Z³ is —(CH)— and Z⁴ is —N—.

In other embodiments of formula III, Z¹ is —N—; Z³ is —C(R⁶)— and Z⁴ is —C(R⁵)—.

In other embodiments of formula III, Z¹ is —N—; Z³ is —N— and Z⁴ is —N—.

In other embodiments of formula III, L¹ is —C(O)C(O)—, and L² is —O—.

In other embodiments of formula III, L¹ is —C(O)—, and L² is —O—.

In other embodiments of formula III, L¹ is —(C₁-C₆)alkyl optionally substituted with one or more groups selected from halo; D, —(C₁-C₃)alkyl optionally substituted with 1-3 groups selected from halo or D; —(C₁-C₃)alkoxy optionally substituted with 1-3 groups independently selected halo or D; or a spiro-(C₃-C₆)cycloalklyl optionally substituted with 1-2 groups selected from halo, D, methyl, or halogenated methyl; and L² is oxycarbonylaryl or oxycarbonylheteroaryl, wherein each aryl or heteroaryl group of L² is optionally substituted with one or more substituents independently selected from halo, D, aryl, —(C₁-C₆)alkyl, hydroxyl, nitro, amino, alkoxy, alkylthio, —CO₂H, and CN.

In another embodiment of formula III, G is absent and Z¹ is N.

In another embodiment of formula III, G is absent; Z¹ is N; and R¹ and R^{1′} are each D.

In another embodiments of the compounds having formulae I or II, G is absent, and Z³ and Z⁴ are each N.

In another embodiment of formula III, G is absent, Z³ and Z⁴ are each N; and R¹ and R^{1′} are each D. In other embodiments of the compound of Formulae I, II or III, or a pharmaceutically acceptable salt, and including deuterated forms thereof G is absent, R¹ and R^{1′} are each D, and one or both of Z¹ and Z³ are selected from —C(H)— or provided that at least one of Z¹ and Z³ is N.

In other embodiments of the compounds having formula III, including the embodiments of formula III described in this specification, R⁷ is selected —OR^{x}, wherein R^{x} is as defined in the specification.

In other embodiments of formulae I, II, III, including the embodiments of formula I, II and II described in this specification, R⁷ is selected from halo, —O—C₁-C₄alkyl optionally substituted with one or more D or halo, —S—(C₁-C₄)alkyl optionally substituted with one or more D or halo, —S(O)—(C₁-C₄)alkyl optionally substituted with one or more D or halo, —S(O)₂—(C₁-C₄)alkyl optionally substituted with one or more D or halo, or —(O)—(C₁-C₄)alkyl optionally substituted with one or more D or halo.

Another embodiment of the compounds of formulae I, II, III relate to one or more compounds of formulae IV(a), IV(b), IV(c), IV(d), IV(e) or IV(f):

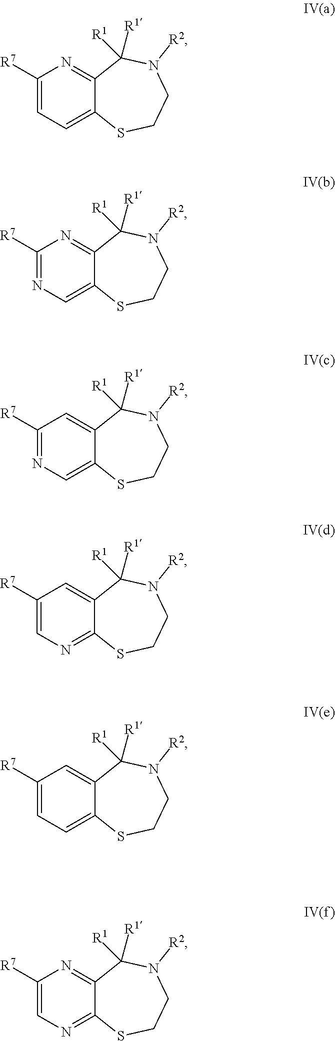

or a pharmaceutically acceptable salt, and including deuterated forms thereof, wherein

R⁷ is —O—(C₁-C₄)alkyl optionally substituted with one to three substituents independently selected from D and halo; —(C₁-C₄)alkyl optionally substituted with one to three substituents independently selected from D and halo, or halo;

R² is -L¹-L²-G;

L¹ is —C(O)C(O)—; or —C(R¹⁰)(R¹¹)—;

L² is —O— or oxycarbonylphenyl optionally substituted with 1-3 substituents independently selected from halo, D, aryl, —(C₁-C₃)alkyl, hydroxyl, nitro, amino, alkoxy, alkylthio, —CO₂H, and CN;

G is absent or an NO donor selected from —(C₁-C₁₀)alkyl substituted with 1 or 2 —ONO₂ groups, —C(H)₂—O—R⁹, —(C₁-C₆)alkylene-O—C(H)₂C(H)(ONO₂)—(C₁-C₆)alkyl, -phenylene-R⁹, —(C₁-C₆)alkylene-S(O)₂N(H)(OH),

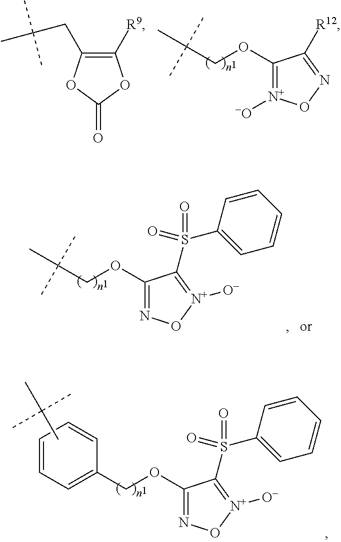

provided that when G is absent, the compound cannot be of formula IV(e);

R⁹ is —(C₂-C₁₀)alkyl substituted with 1 or 2 —ONO₂ groups;

R¹⁰ and R¹¹ are each independently selected from H, D, —CH₃, halogenated methyl, —CD₃, or R¹⁰ and R¹¹ taken together with the carbon to which they are attached join to form a spiro-(C₃-C₆)cycloalklyl optionally substituted with 1-2 groups selected from halo, D, methyl, and halogenated methyl;

R¹² is H or —(C₁-C₃)alkyl and

n¹ is an integer from 0-3,

wherein each alkylene group of G is optionally substituted with 1-2 substituents selected from halo, aryl, hydroxyl, nitro, amino, alkoxy, alkylthio, —CO₂H, and CN.

In other embodiments of formulae I, II, III, IV(a), IV(b), IV(c) IV(d), IV(e) and IV(d), and subembodiments thereof as described above, or a pharmaceutically acceptable salt, and including deuterated forms thereof:

R⁷ is —OMe, —OCD₃, —OCF₃, —O-n-propyl, —O-isopropyl, —O-n-butyl, —O-s-butyl, —O-t-butyl, —O-isobutyl, —O-cylclopropyl, —CD₃ or —CF₃.

In other embodiments of formulae IV(a), IV(b), IV(c), IV(d), IV(e) and IV(f), L¹ is —C(O)C(O)—, and L² is —O—.

In other embodiments of formulae IV(a), IV(b), IV(c), IV(d), IV(e) and IV(f), L¹ is —C(O)—, and L² is —O—.

In other embodiments of formulae IV(a), IV(b), IV(c), IV(d), IV(e) and IV(f), L¹ is —(C₁-C₆)alkyl optionally substituted with one to three groups selected from halo; —(C₁-C₃)alkyl optionally substituted with 1-3 groups selected from halo and D; —(C₁-C₃)alkoxy optionally substituted with 1-3 groups independently selected from halo and D; or a (C₃-C₆)cycloalklyl optionally substituted with 1-2 groups selected from halo, D, methyl, and halogenated methyl; and L² is oxycarbonylaryl or oxycarbonylheteroaryl, wherein each aryl or heteroaryl group of L² is optionally substituted with one to three substituents independently selected from halo, —(C₁-C₆)alkyl, hydroxyl, nitro, amino, alkoxy, alkylthio, —CO₂H, and CN.

In other embodiments of formulae IV(a), IV(b), IV(c), IV(d), IV(e) and IV(f), including the embodiments of any of these formulae described in this specification, R⁷ is selected —OR^{x}, wherein R^{x} is as defined in the specification

In other embodiments of formulae IV(a), IV(b), IV(c), IV(d), IV(e) and IV(f), including the embodiments of any of these formulae described in this specification, R⁷ is selected from halo, —O—C₁-C₄alkyl optionally substituted with one or more halo, —S—(C₁-C₄)alkyl optionally substituted with one or more D or halo, —S(O)—(C₁-C₄)alkyl optionally substituted with one or more D or halo, —S(O)₂—(C₁-C₄)alkyl optionally substituted with one or more D or halo halo, or —(O)—(C₁-C₄)alkyl optionally substituted with one or more D or halo.

Other embodiments of the compounds of formulae I, II, III, IV(a), IV(b), IV(c), IV(d), IV(e) and IV(f) relate to one or more compounds of formulae V(a), V(b), V(c), V(d), V(e) or V(f):

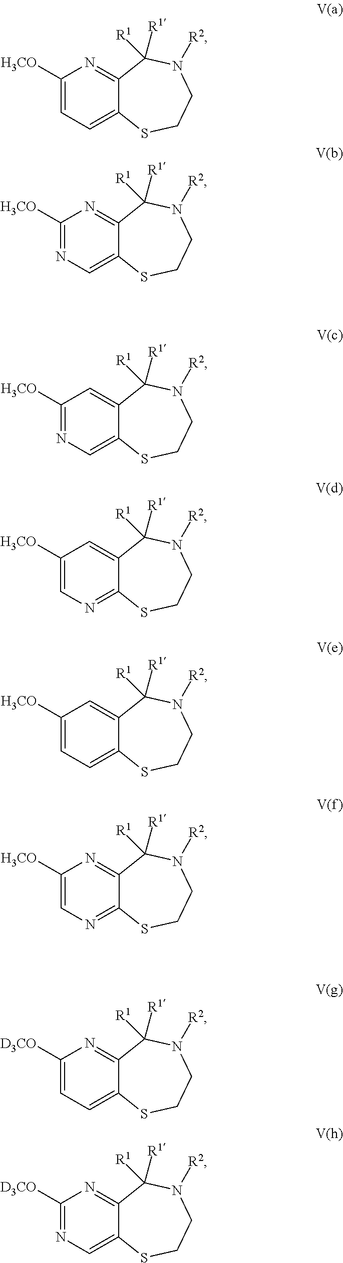

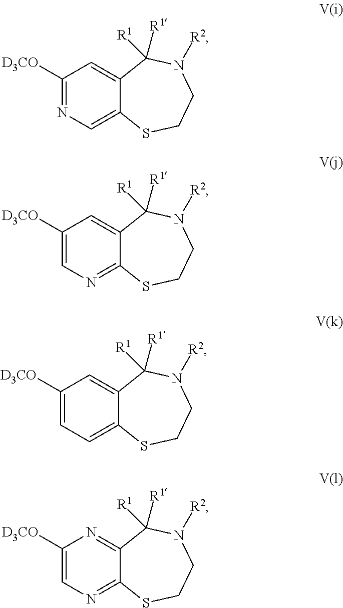

or a pharmaceutically acceptable salt, and including deuterated forms thereof, wherein:

R² is -L¹-L²-G;

L¹ is —C(O)C(O)— or —C(R¹⁰)(R¹¹);

L² is —O—, oxycarbonylaryl or oxycarbonylheteroaryl, wherein the aryl or heteroaryl portions are optionally substituted with 1-2 substituents independently selected from halo, —(C₁-C₃)alkyl, hydroxyl, nitro, amino, alkoxy, alkylthio, —CO₂H, and CN;

G is absent or an NO donor selected from —(C₁-C₁₀)alkyl substituted with 1 or 2 —ONO₂ groups, —C(H)₂—O—R⁹, —(C₁-C₆)alkylene-O—C(H)₂C(H)(ONO₂)—(C₁-C₆)alkyl, -phenylene-R⁹, —(C₁-C₆)alkylene-S(O)₂N(H)(OH),

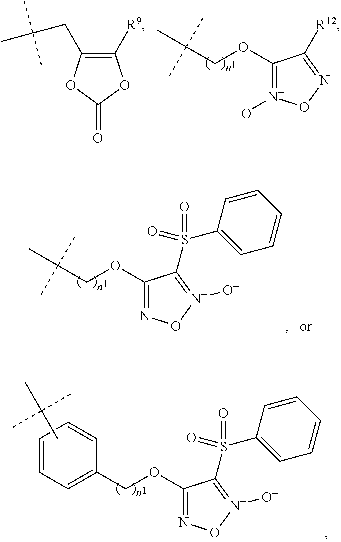

provided that when G is absent, the compound is not of formula V(e);

R⁹ is —(C₂-C₁₀)alkyl substituted with 1 or 2 —ONO₂ groups;

R¹⁰ and R¹¹ are each independently selected from H, D, —CH₃, halogenated methyl, and —CD₃, or R¹⁰ and R¹¹ taken together with the carbon to which they are attached join to form a spiro-(C₃-C₆)cycloalklyl optionally substituted with 1-2 groups selected from halo, D, methyl, and halogenated methyl;

R¹² is H or —(C₁-C₃)alkyl; and

n is an integer from 0-3;

wherein each alkylene group of G is optionally substituted with 1-2 substituents selected from halo, aryl, hydroxyl, amino, alkoxy, and alkylthio.

In other embodiments of formulae V(a), V(b), V(c), V(d), V(e), V(f), V(g), V(h), V(i), V(j), V(k), or V(l), L¹ is —C(O)C(O)—, and L² is —O—.

In other embodiments of formulae V(a), V(b), V(c), V(d), V(e), V(f), V(g), V(h), V(i), V(j), V(k), or V(l), L¹ is —C(O)—, and L² is —O—.

In other embodiments of formulae V(a), V(b), V(c), V(d), V(e), V(f), V(g), V(h), V(i), V(j), V(k), or V(l), L¹ is —(C₁-C₆)alkyl optionally substituted with one or more groups selected from halo; —(C₁-C₃)alkyl optionally substituted with 1-3 groups selected from halo and D; —(C₁-C₃)alkoxy optionally substituted with 1-3 groups independently selected from halo and D; or a spiro-(C₃-C₆)cycloalklyl optionally substituted with 1-2 groups selected from halo, D, methyl, and halogenated methyl; and L² is oxycarbonylaryl or oxycarbonylheteroaryl, wherein each aryl or heteroaryl group of L² is optionally substituted with one to three substituents independently selected from halo, —(C₁-C₆)alkyl, hydroxyl, nitro, amino, alkoxy, alkylthio, —CO₂H, and CN.

In other embodiments of formulae V(a), V(b), V(c), V(d), V(e), V(f), V(g), V(h), V(i), V(j), V(k), or V(l), including the embodiments of any of these formulae described in this specification, R⁷ is selected —OR^{x}, wherein R^{x} is as defined in the specification.

In other embodiments of formulae V(a), V(b), V(c), V(d), V(e), V(f), V(g), V(h), V(i), V(j), V(k), or V(l), including the embodiments of any of these formulae described in this specification, R⁷ is selected from halo, —O—C₁-C₄alkyl optionally substituted with one or more halo, —S—(C₁-C₄)alkyl optionally substituted with one or more halo, —S(O)—(C₁-C₄)alkyl optionally substituted with one or more halo, —S(O)₂—(C₁-C₄)alkyl optionally substituted with one or more halo, or —(O)—(C₁-C₄)alkyl optionally substituted with one or more halo.

In other embodiments of formulae I, II, III, IV(a), IV(b), IV(c), IV(d), IV(e), IV(f), V(a), V(b), V(c), V(d), V(e), V(f), V(g), V(h), V(i), V(j), V(k), or V(l), and subembodiments thereof as described above, or a pharmaceutically acceptable salt, and including deuterated forms thereof:

R² is

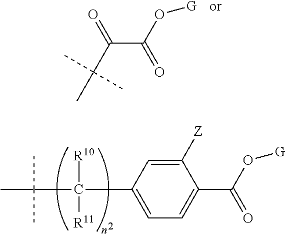

G is an NO donor selected from C₁₋₁₀alkyl substituted with 1 or 2 —ONO₂ or

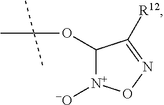

R¹² is H or CH₃;

R¹⁰ and R¹¹ are each independently selected from H, D, —CH₃, halogenated methyl, and —CD₃, or R¹⁰ and R¹¹ taken together with the carbon to which they are attached join to form a cyclopropyl;

Z is H, halo or —(C₁-C₃)alkoxy, and

n² is an integer from 1-2.

The ortho substituted Z group is beneficial and advantageous over the teachings in the art by being able to reduce the clearance of the compounds of the invention (leading to better or longer exposure) by forming an intra-molecular hydrogen bond with the proton of the acid group.

In other embodiments of formulae I, II, III, IV(a), IV(b), IV(c), IV(d), IV(e) or IV(f), V(a), V(b), V(c), V(d), V(e), V(f), V(g), V(h), V(i), V(j), V(k), or V(l), and subembodiments thereof as described above, or a pharmaceutically acceptable salt, and including deuterated forms thereof:

R² is

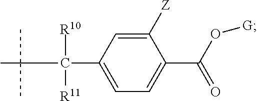

and

R¹⁰ and R¹¹ are each independently selected from H, D, —CH₃, halogenated methyl, and —CD₃; or R¹⁰ and R¹¹ taken together with the carbon to which they are attached join to form a yelopropyl;

G is absent or C₁₋₁₀alkyl substituted with 1 or 2 —ONO₂, provided that when G is absent (or H), the compound cannot be of Formula IV(e) or V(e); and

Z is H, fluoro or methoxy.

In other embodiments of formulae I, II, III, IV(a), IV(b), IV(c), IV(d), IV(e), IV(f), V(a), V(b), V(c), V(d), V(e), V(f), V(g), V(h), V(i), V(j), V(k), or V(l), and subembodiments thereof as described above, or a pharmaceutically acceptable salt, and including deuterated forms thereof:

R² is

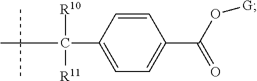

and.

R¹⁰ and R¹¹ are each independently selected from H, D, —CH₃, halogenated methyl, and —CD₃, or R¹⁰ and R¹¹ taken together with the carbon to which they are attached join to form a cyclopropyl;

G is hydrogen or C₁₋₁₀alkyl substituted with 1 or 2 —ONO₂, provided that when G is hydrogen, the compound cannot be of Formula IV(e) or V(e); and

Z is H, fluoro or methoxy.

In other embodiments of formulae I, II, III, IV(a), IV(b), IV(c), IV(d), IV(e) or IV(f), V(a), V(b), V(c), V(d), V(e) or V(f), and subembodiments thereof as described above, or a pharmaceutically acceptable salt, and including deuterated forms thereof:

R² is

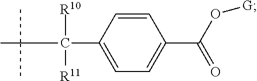

and.

R¹⁰ and R¹¹ are each independently selected from H, D, —CH₃, halogenated methyl, —CD₃, or R¹⁰ and R¹¹ taken together with the carbon to which they are attached join to form a cyclopropyl;

G is hydrogen or C₁₋₁₀alkyl substituted with 1 or 2 —ONO₂, provided that when G is hydrogen, the compound cannot be of Formula IV(e) or V(e); and

Z is fluoro or methoxy.

In other embodiments of formulae I, II, III, IV(a), IV(b), IV(c), IV(d), IV(e)r IV(f), V(a), V(b), V(c), V(d), V(e), V(f), V(g), V(h), V(i), V(j), V(k), or V(l), and subembodiments thereof as described above, or a pharmaceutically acceptable salt, and including deuterated forms thereof:

R² is

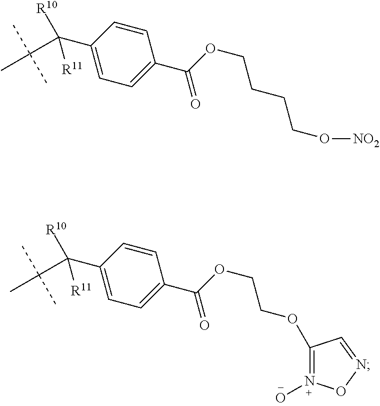

and
R¹⁰ and R¹¹ are each independently selected from H, D, —CH₃, and —CD₃, or R¹⁰ and R¹¹ join together to form a cyclopropyl.

### Further Embodiments of G

All of the embodiments of G described below are exemplary in nature and can be incorporated in any of the above formula, and embodiments thereof, wherever applicable.

Non-limiting examples of —(C₁-C₁₀)alkyl substituted include with 1 or 2 —ONO₂ include —(C₁-C₆)alkyl substituted with 1 —ONO₂. Other examples of —(C₁-C₁₀) alkyl substituted include with 1 or 2 —ONO₂ include —(C₁-C₆)alkyl with 2 —ONO₂.

Other non-limiting examples of —(C₁-C₁₀)alkyl substituted include with 1 or 2 —ONO₂ include —(C₁-C₆)alkyl substituted with 1 or 2 —ONO₂. Other examples of —(C₁-C₆)alkyl substituted include with 1 or 2 —ONO₂ include —(C₁-C₆)alkyl substituted with 1 —ONO₂. Other examples of —(C₁-C₆) alkyl substituted include with 1 or 2 —ONO₂ include —(C₁-C₆)alkyl with 2 —ONO₂.

Non-limiting examples of —(C₁-C₆)alkyl substituted include with 1 —ONO₂ include —CH₂—ONO₂, —(CH₂)₂ONO₂, —(CH₂)₃—ONO₂, —(CH₂)₅ONO₂, and (CH₂)₆—ONO₂.

Non-limiting examples of —(C₁-C₆)alkyl substituted include with 2 —ONO₂ include —(CH₂)₂(ONO₂)CH₂(ONO₂), —(CH₂)₃(ONO₂)CH₂(ONO₂), and —(CH₂)₂CH(ONO₂)CH(ONO₂) CH₃.

Non-limiting examples of -phenylene-R⁹ examples include:

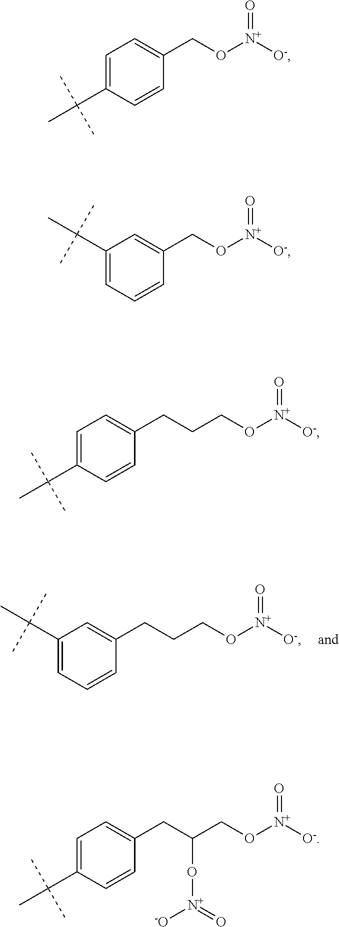

Non-limiting examples of —(C₁-C₆)alkylene-SO₂NH(OH) moieties include

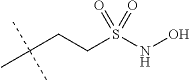

Non-limiting examples of

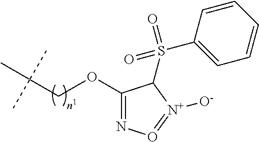

wherein n¹ can be 0-5 include:

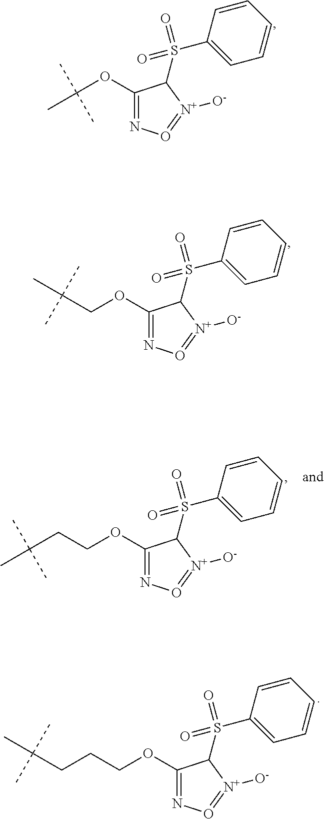

Non-limiting examples of

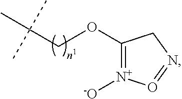

wherein n¹ is 0-5 include:

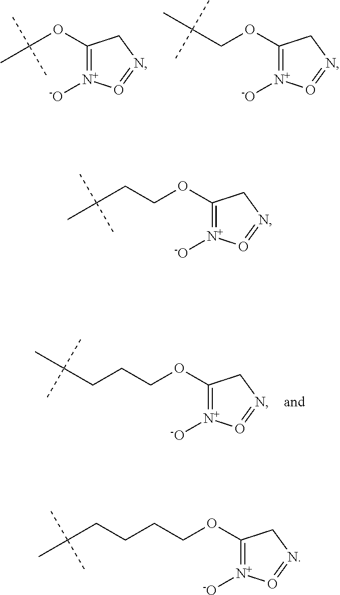

Other embodiments of NO donators that can be used for G can be obtained from WO 2013/181332, which is incorporated herein by references.

In another embodiment of formula I, and subembodiments thereof as described above, relate to any one or more of the following compounds:

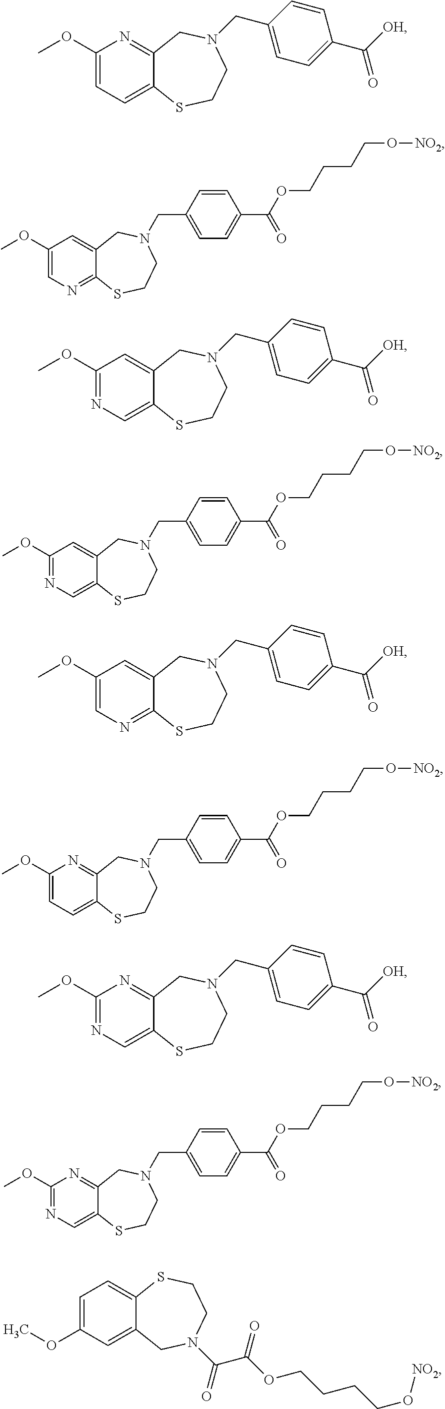

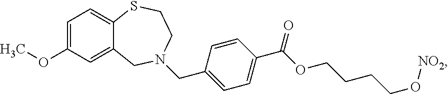 [SMILES: COc1ccc2c(c1)CN(Cc1ccc(C(=O)OCCCCO[N+](=O)[O-])cc1)CCS2]

or a pharmaceutically acceptable salt of any of the above compounds, including deuterated forms thereof.

Another aspect of the invention relates to a compound of formulae VI:

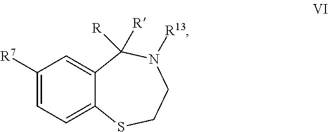

or a pharmaceutically acceptable salt, and including deuterated forms thereof, wherein:

each of R and R′ is H or D;

R⁷ is selected from halo, D, —O—(C₁-C₄)alkyl optionally substituted with 1-3 members selected from halo and D, and —(C₁-C₄)alkyl optionally substituted with 1-3 members selected from halo and D;

R¹³ is -L³-L⁴

L³ is —C(R¹⁰)(R¹¹)—;

L⁴ is oxycarbonylphenyl optionally substituted with 1-3 substituents independently selected from halo, aryl, —(C₁-C₃)alkyl, hydroxyl nitro, amino, alkoxy, alkylthio, —CO₂H, and CN;

R¹⁰ and R¹¹ are each independently selected from H, D, —CH₃, halogenated methyl and —CD₃, or R¹⁰ and R¹¹ taken together with the carbon to which they are attached join to form a spiro-(C₃-C₆)cycloalkyl optionally substituted with 1-2 groups selected from halo, D, methyl, or halogenated methyl, provided that R¹⁰ and R¹¹ cannot both be H.

In another embodiment of formulae VI,

R and R′ are each D; and

R⁷ is —OMe, —OCD₃, —OCF₃, —O-n-propyl, —O-isopropyl, —O-n-butyl, —O-s-butyl, —O-t-butyl, —O-isobutyl, —O-cylclopropyl, —CD₃ or —CF₃.

Another embodiment of formulae VII:

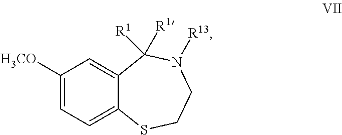

or a pharmaceutically acceptable salt, and including deuterated forms thereof, wherein:

each of R and R′ is H or D;

R¹³ is -L³-L⁴

L³ is —C(R¹⁰)(R¹¹);

L⁴ is oxycarbonylphenyl; and

R¹⁰ and R¹¹ are each independently selected from H, D, —CH₃, halogenated methyl, —CD₃, or R¹⁰ and R¹¹ taken together with the carbon to which they are attached join to form a spiro-(C₃-C₆)cycloalklyl optionally substituted with 1-2 groups selected from halo, D, methyl, or halogenated methyl, provided that R¹⁰ and R¹¹ cannot both be H.

Another aspect of the invention relates to a salt of any of the compounds described above, wherein the salt is selected from sodium, potassium, magnesium, hemifumarate, hydrochloride or hydrobromide.

Another aspect of the invention relates to a pharmaceutical composition comprising any of the compounds described above in combination with optionally one or more pharmaceutically acceptable excipients or carriers.

Another aspect of the invention relates to a pharmaceutical composition comprising a compound comprising any of the compounds described above in combination with one or more NO donors and with one or more pharmaceutically acceptable excipients or carriers.

Another aspect of the invention relates to methods of treating or preventing muscle disorders, diseases and conditions associated with dysfunctions in calcium modulation, comprising administering to a subject in need of such treatment an amount of a compound or pharmaceutical composition, as described in the specification, to effectuate such treatment.

Another aspect of the invention relates to a compound, or pharmaceutical compositions thereof, as described in the specification, optionally in combination with an NO donor as described in the specification, for use in the treatment or prevention of various muscle disorders, diseases and conditions associated with dysfunctions in calcium homeostasis or modulation, comprising administering to a subject in need of such treatment an amount of a compound or pharmaceutical composition, as described in the specification, effective to prevent or treat the disorder, disease or condition associated with a dysfunctions in calcium homeostasis or modulation.

Another aspect of the invention relates to any of the compounds described above, or any of the pharmaceutical compositions described above, for use in the treatment or prevention of a condition selected from cardiac disorders and diseases, muscle fatigue, musculoskeletal disorders and diseases, diseases associated with colon function, CNS disorders and diseases, cognitive dysfunction, neuromuscular disorders and diseases, bone disorders and diseases, cancer cachexia, malignant hyperthermia, diabetes, sudden cardiac death, sudden infant death syndrome, or for improving cognitive function.

Another aspect of the invention relates to a method of treating or preventing a condition selected from cardiac disorders and diseases, muscle fatigue, musculoskeletal disorders and diseases, diseases associated with colon function, CNS disorders and diseases, cognitive dysfunction, neuromuscular disorders and diseases, bone disorders and diseases, cancer cachexia, malignant hyperthermia, diabetes, sudden cardiac death, and sudden infant death syndrome, or for improving cognitive function, the method comprising administering to a patient in need thereof a therapeutically effective amount of any of the compounds described above, or any of the pharmaceutical compositions described above, to effectuate such treatment.

In another embodiment of the uses and methods described above, the condition is associated with an abnormal calcium homeostasis or modulation.

In another embodiment of the uses and methods described above, the condition is associated with an abnormal function of a ryanodine receptor.

In another embodiment of the uses and methods described above, the cardiac disorders and diseases are selected from irregular heartbeat disorders, atrial and ventricular arrhythmia, atrial and ventricular fibrillation, atrial and ventricular tachyarrhythmia, atrial and ventricular tachycardia, catecholaminergic polymorphic ventricular tachycardia (CPVT), exercise-induced irregular heartbeat disorders and diseases, congestive heart failure, chronic heart failure, acute heart failure, systolic heart failure, diastolic heart failure, acute decompensated heart failure, cardiac ischemia/reperfusion (FR) injury, chronic obstructive pulmonary disease, I/R injury following coronary angioplasty or following thrombolysis for the treatment of myocardial infarction (MI), or high blood pressure.

In another embodiment of the uses and methods described above, the musculoskeletal disorder, disease or condition is selected from exercise-induced skeletal muscle fatigue, exercise-induced muscle fatigue, a congenital myopathy, central core disease (CCD), Lambert-Eaton myastenic syndrome, Duchenne Muscular Dystrophy (DMD), Becker's Muscular Dystrophy (BMD), Limb-Girdle Muscular Dystrophy (LGMD) and its subtypes such as LGMD1 subtypes A throught H (subtypes A, B, C, D, E, F, G and H) and LGMD2 subtypes A through Q (subtype A, B, C, D, E, F, G, H, I, J, K L, M, N O and Q), facioscapulohumeral dystrophy (FSHD), Friedreich's ataxia (FA), inclusion-body myositis, myotonic muscular dystrophy, hyperthyroid myopathy, congenital muscular dystrophy (CMD), distal muscular dystrophy, inflammatory myositis, Emery-Dreifuss muscular dystrophy, oculopharyngeal muscular dystrophy, myasthenia gravis, rippling muscle disease, mitochondrial myopathies, Ryanodine-related myopathies, spinal muscular atrophy (SMA), Spinal and bulbar muscular atrophy (SBMA), age-related muscle fatigue, sarcopenia, bladder disorders, or incontinence.

In another embodiment of the uses and methods described above, the musculoskeletal disorder, disease or condition is exercise-induced skeletal muscle fatigue.

In another embodiment of the uses and methods described above, the musculoskeletal disorder, disease or condition is a congenital myopathy.

In another embodiment of the uses and methods described above, the musculoskeletal disorder, disease or condition is Duchenne Muscular Dystrophy (DMD).

In another embodiment of the uses and methods described above, the musculoskeletal disorder, disease or condition is Becker's Muscular Dystrophy (BMD).

In another embodiment of the uses and methods described above, the musculoskeletal disorder, disease or condition is Limb-Girdle Muscular Dystrophy (LGMD).

In another embodiment of the uses and methods described above, the musculoskeletal disorder, disease or condition is facioscapulohumeral dystrophy (FSHD).

In another embodiment of the uses and methods described above, the musculoskeletal disorder, disease or condition is myotonic muscular dystrophy. In another embodiment of the uses and methods described above, the musculoskeletal disorder, disease or condition is congenital muscular dystrophy (CMD).

In another embodiment of the uses and methods described above, the musculoskeletal disorder, disease or condition is distal muscular dystrophy.

In another embodiment of the uses and methods described above, the musculoskeletal disorder, disease or condition is Emery-Dreifuss muscular dystrophy.

In another embodiment of the uses and methods described above, the musculoskeletal disorder, disease or condition is oculopharyngeal muscular dystrophy.

In another embodiment of the uses and methods described above, the musculoskeletal disorder, disease or condition is spinal muscular atrophy (SMA).

In another embodiment of the uses and methods described above, the musculoskeletal disorder, disease or condition is Spinal and bulbar muscular atrophy (SBMA).

In another embodiment of the uses and methods described above, the musculoskeletal disorder, disease or condition is age-related muscle fatigue.

In another embodiment of the uses and methods described above, the musculoskeletal disorder, disease or condition is sarcopenia.

In another embodiment of the uses and methods described above, the musculoskeletal disorder, disease or condition is central core disease.

In another embodiment of the uses and methods described above, the musculoskeletal disorder, disease or condition is bladder disorders.

In another embodiment of the uses and methods described above, the musculoskeletal disorder, disease or condition is and incontinence. In another embodiment of the uses and methods described above the CNS disorders and diseases are selected from Alzheimer's Disease (AD), neuropathy, seizures, Parkinson's Disease (PD), or Huntington's Disease (HD); and the neuromuscular disorders and diseases are selected from Spinocerebellar ataxia (SCA), or Amyotrophic lateral sclerosis (ALS, Lou Gehrig's disease).

In another embodiment of the uses and methods described above, the condition that can be treated with the compounds or compositions described herein is a disease or condition associated with colon function.

Another aspect of the invention relates to method for treating a subject that has Duchenne Muscular Dystrophy (DMD), comprising the step of administering to said subject an amount of a compound, or pharmaceutical composition thereof, according to any of the embodiments described above, in combination with an antisense oligonucleotide (AO) which is specific for a splicing sequence of at least one exon of the DMD gene; a steroid such as prednisone, deflazacort or the like; a myostatin (GDF-8) antibody (e.g. PF-06252616, BMS-986089, LY2495655 or the like; folliststin gene therapy; micro and mini dystrophin gene (AAV) therapy; micro and mini utrophin gene (AAV) therapy; an upregulator of utrophin expression such as SMT C1100 and the like; anti-fibrotic agents such as halofuginone, FG-3019, BG00011 (STX-100) and the like; a stop-codon (or nonsence) readthrough agent such as PTC124, ataluren, aminoglycoside antibiotics and the like, or human growth factor. In another embodiment of this aspect, the splicing sequence is of exon 23, 45, 44, 50, 51, 52 and/or 53 of the DMD gene.

Another aspect of the invention relates to any of the compounds described above, or pharmaceutical composition thereof, for use in the treatment or prevention of a condition selected from various muscle disorders, diseases and conditions associated with dysfunctions in either NO or calcium modulation.

Another aspect of the invention relates to any of the compounds described above, or pharmaceutical composition thereof, for use in the treatment or prevention of a condition selected from various muscle disorders, diseases and conditions associated with dysfunction in calcium homeostasis or modulation.

Another aspect of the invention relates to methods of treating or preventing various muscle disorders, diseases and conditions associated with dysfunctions in both NO and calcium homeostasis or modulation, comprising administering to a subject in need of such treatment an amount of a compound, as described in the specification, effective to prevent or treat the disorder, disease or condition associated with a dysfunction in both NO and calcium homeostasis or modulation.

### Methods of Synthesis

Compounds of the present invention are, subsequent to their preparation, preferably isolated and purified to obtain a composition containing an amount by weight equal to or greater than about 90% of the compound, about 95% of the compound, and even more preferably greater than about 99% of the compound (“substantially pure” compound) which is then used or formulated as described herein. Such “substantially pure” compounds of the present invention are also contemplated herein as part of the present invention.

Some abbreviations that may appear in this application are as follows.

### DCM Dichloromethane

### DIEA N,N-Diisopropylethylamine

### DME Dimethoxyethane

### DMF Dimethylformamide

EDCI 1-Ethyl-3-(3-dimethylaminopropyl)carbodiimide
IPA Isopropyl alcohol
LAD Lithium aluminum deuteride
LAH Lithium aluminum hydride
TFA Trifluoroacetic acid

### THF Tetrahydrofuran

### EXAMPLES

The examples and schemes below depict the general synthetic procedure for the compounds disclosed herein. Synthesis of the compounds disclosed herein, and embodiments thereof, are not limited by these examples and schemes. One skilled in the art will know that other procedures can be used to synthesize any of the compounds described herein, and that the procedures described in the examples and schemes below are only exemplary procedures. In the descriptions below, one of ordinary skill in the art would recognize that specific reaction conditions, added reagents, solvents, and reaction temperatures can be modified for the synthesis of specific compounds that fall within the scope of this disclosure.

General Synthetic Scheme for Compounds of Formula A

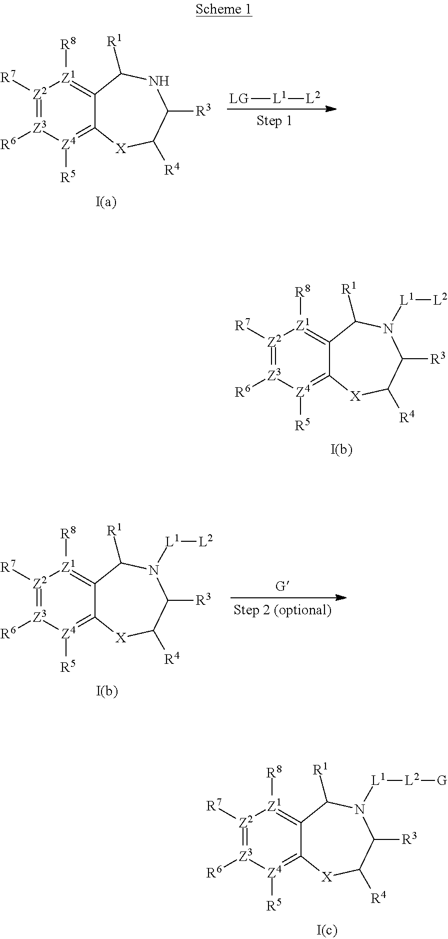

In Scheme 1, LG is a leaving group. Variables R¹, R³, R⁴, R⁵, R⁶, R⁷, R⁸, Z¹, Z², Z³, Z⁴, L¹, L² and X in formula I(a), I(b) and I(c) are as defined in formula I, and embodiments thereof, within the specification. Non-limiting examples of leaving groups include halo, mesylates, tosylates, sulfonates, and the like.

The compounds of formula I(a) can be prepared according the routes of synthesis set forth below, as well as by utilizing methods known in the art, and by making any necessary modifications to any of the starting materials and/or reagents as understood by the skilled medicinal chemist. Additional methods that can be utilized and modified by the skilled medicinal chemist to make compounds of formula I(a) are disclosed in WO 2007/049572 and WO 2008/144483, the contents of which are incorporated herein by reference.

Methods of making NO donating groups (G) and adding them to other moieties are also known in the art, and such methods are described in publications such as WO 2013/181332, the contents of which are incorporated herein by reference.

In Step 1 of Scheme 1, the compound of formula I(a) can be reacted with suitable reagents, such as an alkylating agent in the presence of a base, to yield the compound of Formula I(b). The base can include, without limitation, metal hydrides, N,N-diisopropylethylamine, organic bases such as tertiary amines or aromatic amines. The reaction can also be conducted with a solvent such as, by way of example, one or more of DMF, THF, toluene, acetonitrile, chloroform, dichloromethane, and the like. Alternatively, L¹-L^{2′} can be added to formula 1(a) by the process of reductive amination by alkylating L¹-L^{2′} to formula 1(a) when L¹ is in the form of an aldehyde or ketone and LG is absent. Typical reductive amination conditions can be used with a reducing agent, such as sodium triacetoxyborohydride as a non-limiting example, to produce formula 1(b). In one example wherein L¹ is CH2 or CHD, H—C(O)-L^{2′} can be alkylated to formula 1(a) in the presence of a reducing agent to produce formula 1(b).

The compound of Formula I(b) includes compounds of the invention. Alternatively, formula I(b) can be further reacted, if necessary, to yield the compounds of the invention. Such modification may include, by way of example, conjugation, esterification, alkylation, or hydrolyzation, as well as salt formation by reacting the compound of formula I(b) with a suitable acid or base. Another non-limiting example of a modification may include converting a nitrile precursor into a carboxylic acid by hydrolysis, or into a tetrazole by using sodium azide under suitable conditions. A further non-limiting example of a modification may include converting a carboxylic acid derivative to an ester derivative or an amide derivative or the like.

In Step 2 of Scheme 1, L^{2′} contains a chemical group that can react with G′ to give -L²-G in formula I(c). As a non-limiting example, L² can have a free carboxylic acid group that can esterify to an —OH group on G′, or alternatively L2′ may form an amide moiety by reaction with a free —NH2 or —NH group on G′. As stated, G′ can include, without limitation an —OH group that can esterify to a carboxylic acid group on L² to form -L²-G.

Compounds of the present invention may be prepared according to the general routes of synthesis set forth below, or by making any necessary substitutions of starting materials and/or reagents, as would be understood by the skilled medicinal chemist, to arrive at the compounds of the invention.

### Synthetic Examples

Intermediate compounds such as 7-methoxy-2,3,4,5-tetrahydropyrido[3,2-f][1,4]thiazepine may be prepared as set forth in Scheme 2. A skilled medicinal chemist would understand that compounds with other substitutions at the 7 position can be prepared by starting with a suitably substituted pyridine. Methyl 2-((2-((tert-butoxycarbonyl)amino)ethyl)thio)-5-methoxynicotinate may be prepared by reacting tert-butyl (2-mercaptoethyl)carbamate with methyl 2-chloro-5-methoxynicotinate in the presence of a base such as cesium carbonate in a polar aprotic solvent such as DMF. Intermediate 2 of Scheme 2 may be deprotected by typical methods including treating it with hydrochloride acid to produce intermediate 3. Intermediate 3 of Scheme 2 may be hydrolyzed to the carboxylic acid by techniques well known to one skilled in the art such as with treatment with a base such as lithium hydroxide. Cyclization to intermediate 4 may be afforded treating intermediate 4 with a suitable coupling agent capable of forming an amide bond between a carboxylic acid and an amine, such as 1-ethyl-3-(3-dimethylaminopropyl)carbodiimide (EDCI). Intermediate 4 may be reduced using a suitable reducing agent such as lithium aluminum hydride to give intermediate 5 which may be further reacted as shown in Scheme 1 to produce compounds of the invention. Intermediate 4 may also be reduced to a deuterated form of intermediate 5 using a suitable reducing agent such as, for example, lithium aluminum deuteride, and deuterated intermediate 5 may be further reacted as shown in Scheme 1 to produce deuterated compounds of the invention. Furthermore, it is expected and understood that other similarly substituted 2-chloronicotinic acid methyl ester derivatives such as: 2-chloro-4-methylnicotinic acid methyl ester; methyl 2-chloro-5-methylnicotinate; 2-chloro-5-cyclopropyl-3-pyridinecarboxylic acid methyl ester; 2-chloro-5-(trifluoromethyl)-3-pyridinecarboxylic acid methyl ester; 2-chloro-5-(difluoromethoxy)-3-pyridinecarboxylic acid methyl ester and the like could be employed in Scheme 2 and Scheme 3 to produce novel substituted 2,3,4,5-tetrahydropyrido[3,2-f][1,4]thiazepine derivatives.

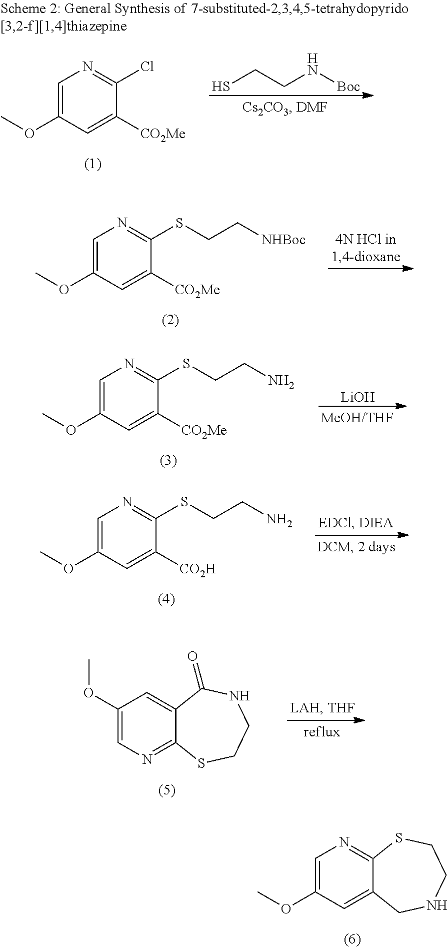

Alternatively, 2,3,4,5-tetrahydropyrido[3,2-f][1,4]thiazepines similar to intermediate 6 of Scheme 2 may be prepared according to the method outlined by Matsumoto et al (WO2009063993). Intermediate 6 of Scheme 3 may be debenzylated by various methods such as by using a palladium catalyst on carbon under an atmosphere of hydrogen to produce intermediate 7. Intermediate 7 of Scheme 3 may be further reacted as shown in Scheme 1 to produce compounds of the invention.

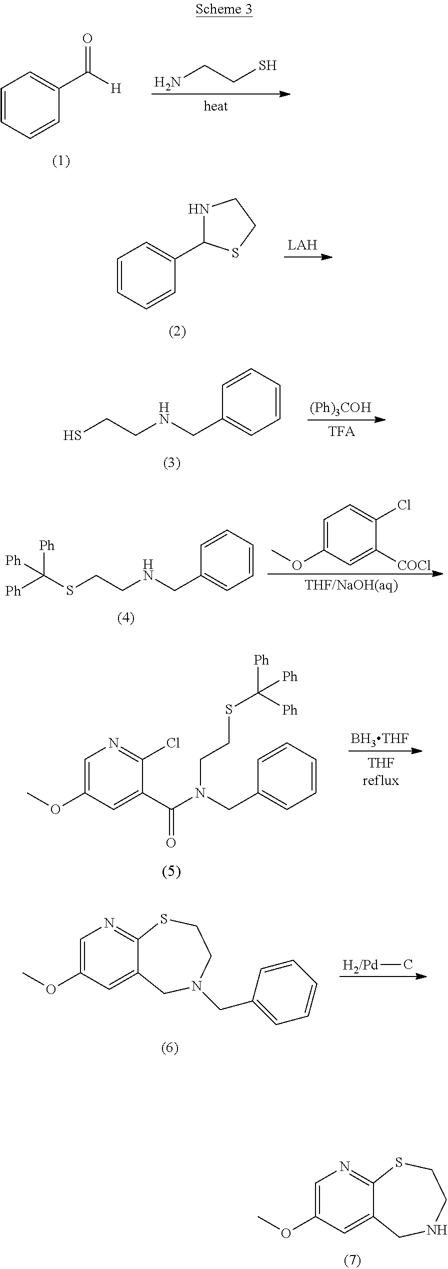

In a similar manner, compounds such as 7-methoxy-2,3,4,5-tetrahydropyrido[4,3-f][1,4]thiazepine may be prepared as set forth in Scheme 4. Compounds such as 4-((7-methoxy-2,3-dihydropyrido[2,3-f][1,4]thiazepin-4(5H)-yl)methyl)benzoic acid may be prepared as set forth in Scheme 5. A skilled medicinal chemist would understand that compounds with other substitutions at the 7 position can be prepared by starting with suitably substituted pyridines in Schemes 3 and 4.

Compounds such as 2-methoxy-6,7,8,9-tetrahydropyrimido[4,5-f][1,4]thiazepine may be prepared as set forth in Scheme 6. A skilled medicinal chemist would understand that compounds with other substitutions at the 2 position can be prepared by starting with a suitably substituted pyrimidine. Intermediate 2 of Scheme 6 may be acylated with dimethyl oxalate to produce intermediate 4 in a manner similar to that set forth by Regan et. al. (Synlett, 23(3), 443-447, 2012). Intermediate 3 may be reacted with tert-butyl (2-mercaptoethyl)carbamate in the presence of a copper catalyst to afford intermediate 4. Deprotection and cyclization strategies similar to those of Schemes 4 and 5 may be utilized to afford 2-substituted-6,7,8,9-tetrahydropyrimido[4,5-f][1,4]thiazepines.

Deuterated examples of the invention may be prepared in a manner as illustrated in Schemes 1 and 7. 7-Methoxy-3,4-dihydrobenzo[f][1,4]thiazepin-5(2H)-one may be reacted with lithium aluminum deuteride by methods known to one skilled in the art to produce 5,5-dideutero-7-methoxy-2,3,4,5-tetrahydrobenzo[f][1,4]thiazepine. This compound may be reacted as shown in Scheme 1 to prepare compounds of the invention. As a non-limiting example, 5-dideutero-7-methoxy-2,3,4,5-tetrahydrobenzo[f][1,4]thiazepine of Scheme 7 may be further reacted with methyl 4-formylbenzoate in the presence of sodium triacetoxyborodeuteride to produce 4-((5,5-dideutero-7-methoxy-2,3-dihydrobenzo[f][1,4]thiazepin-4(5H)-yl)deuteromethyl)benzoic acid. A skilled medicinal chemist would understand that compounds with other substitutions at the 7 position can be prepared by starting with a suitably substituted 3,4-dihydrobenzo[f][1,4]thiazepin-5(2H)-one and using the synthetic routes of Schemes 7 and 8.

Additional deuterated examples of the invention may be prepared by the general synthesis illustrated in Scheme 9. 7-methoxy-3,4-dihydrobenzo[f][1,4]thiazepin-5(2H)-one may be demethylated by procedures known to one skilled in the art such as with the use of boron tribromide. This intermediate 2 of Scheme 9 may then be alkylated with a deuterated reagent such as deuterated iodomethane to produce the deuterated intermediate 3 of Scheme 9. Intermediate 3 of Scheme 9 may be reduced with a suitable reducing agent such as lithium aluminum hydride to produce intermediate 4 of Scheme 9 which may then be reacted with conditions shown in Scheme 1 to produce compounds of the invention.

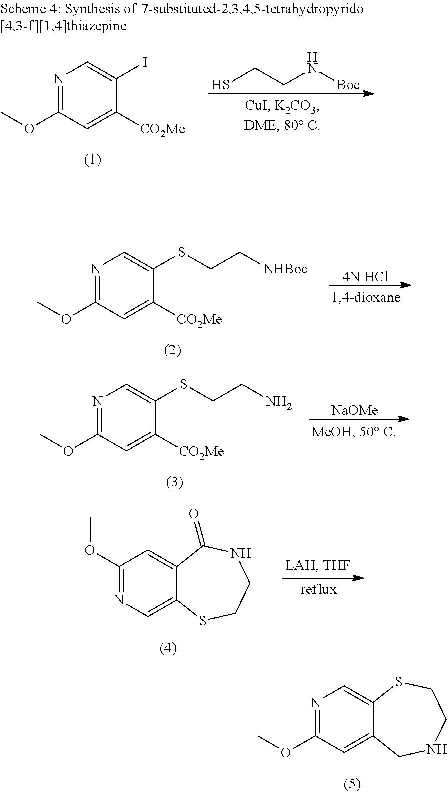

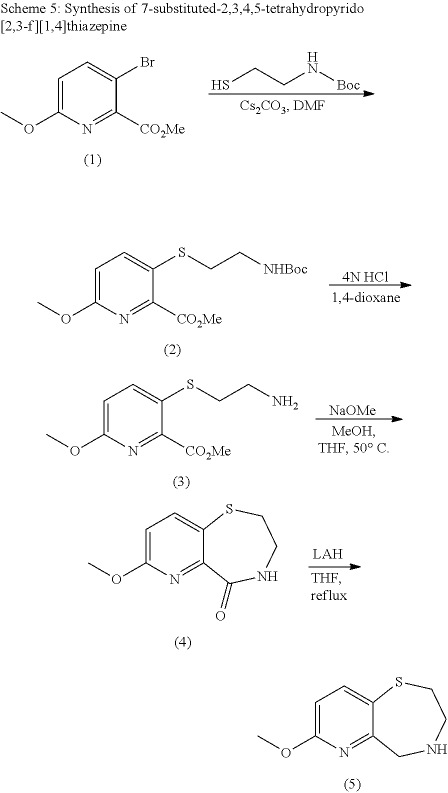

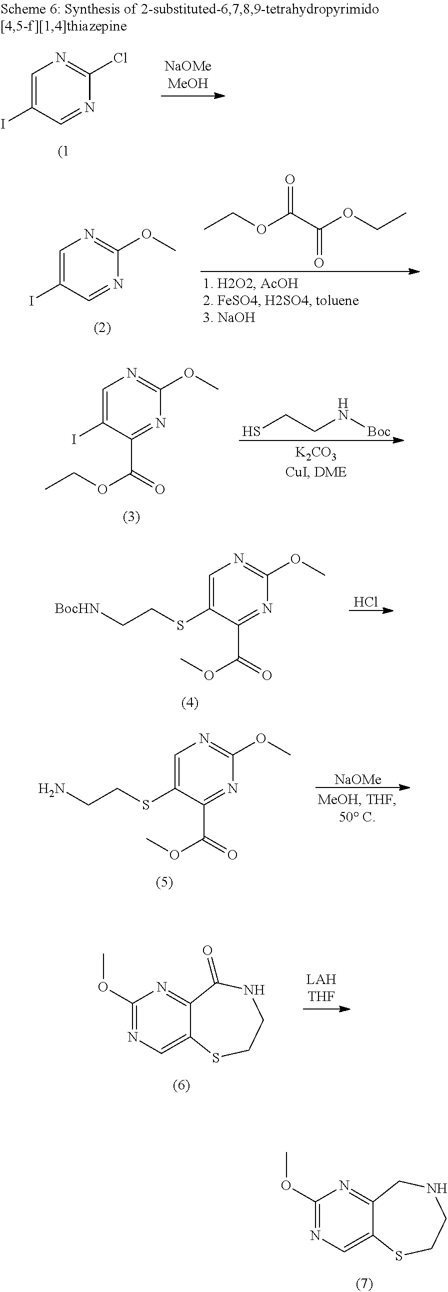

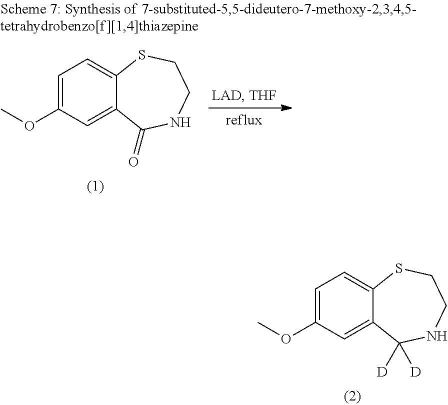

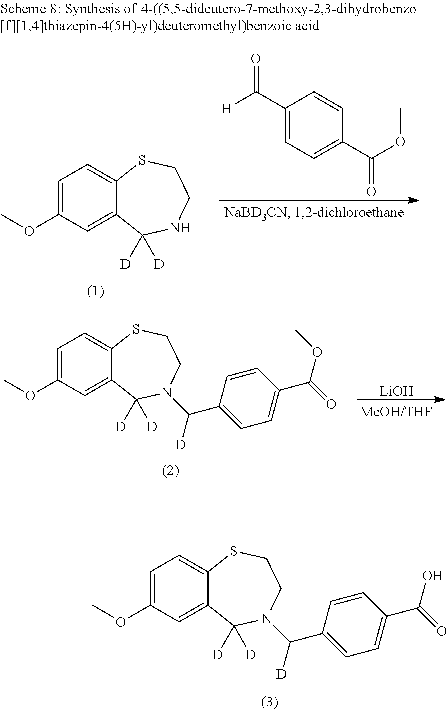

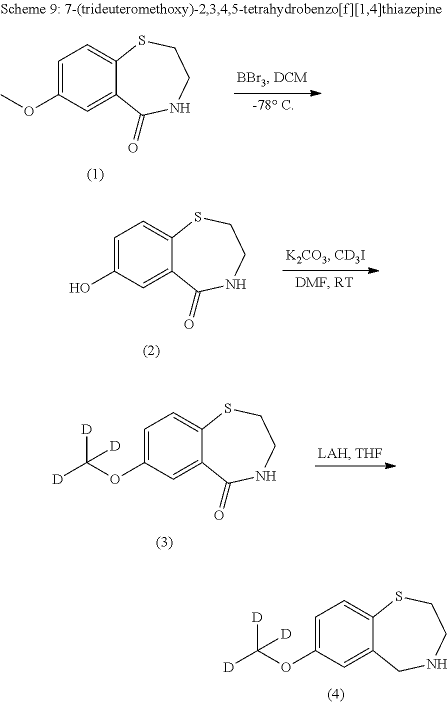 [SMILES: COc1ccc2c(c1)C(=O)NCCS2 ; O=C1NCCSc2ccc(O)cc21 ; [2H]C([2H])([2H])Oc1ccc2c(c1)C(=O)NCCS2 ; [2H]C([2H])([2H])Oc1ccc2c(c1)CNCCS2]

The examples presented below are intended to illustrate particular aspects of the invention and are not intended to limit the scope of the specification or the claims in any way.

### Example 1

### 4-((5,5-Dideutero-7-methoxy-2,3-dihydrobenzo[f][1,4]thiazepin-4(5H)-yl)deuteromethyl)benzoic acid

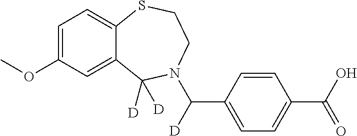 [SMILES: [2H]C(c1ccc(C(=O)O)cc1)N1CCSc2ccc(OC)cc2C1([2H])[2H]]

### Step 1: 5,5-Dideutero-7-methoxy-2,3,4,5-tetrahydrobenzo[f][1,4]thiazepine

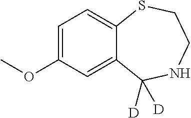

A round bottom flask was charged with lithium aluminum deuteride (0.802 g, 19.1 mmol, 2.0 equiv.), THF (20 mL) and 7-methoxy-3,4-dihydrobenzo[f][1,4]thiazepin-5(2H)-one, which was prepared according to the procedure given in WO2009026444, (2.000 g, 9.6 mmol, 1.0 equiv.) in THF (20 ml) was added. The reaction mixture was heated to reflux under nitrogen overnight. The reaction mixture was cooled to 0° C. and quenched with a few drops of water and stirred for 30 minutes and filtered over celite. The celite cake was washed with ethyl acetate repeatedly. The organic solvents were concentrated to dryness under reduced pressure to give the product as a white solid (1.75 g, 92.8%) which was used without further purification. LC/MS: 198.1 [M+1]⁺. 1H NMR (300 MHz, CDCl3): δ 7.47 (d, J=8.4 Hz, 1H), 6.79 (d, J=3.0 Hz, 1H), 6.68 (dd, J=2.7 and 8.7 Hz, 1H), 3.79 (s, 3H), 3.40-3.37 (m, 2H), 2.71-2.67 (m, 2H).

### Step 2: Methyl 4-((5,5-dideutero-7-methoxy-2,3-dihydrobenzo[f][1,4]thiazepin-4(5H)-yl)deuteromethyl)benzoate

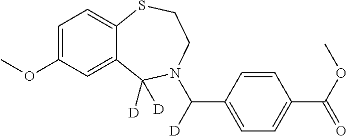 [SMILES: [2H]C(c1ccc(C(=O)OC)cc1)N1CCSc2ccc(OC)cc2C1([2H])[2H]]

A round bottom flask was charged with 5,5-dideutero-7-methoxy-2,3,4,5-tetrahydrobenzo[f][1,4]thiazepine (1.75 g, 8.8 mmoles), methyl 4-formyl benzoate (5.82 g, 35.48 mmoles) and 1,2-dichloroethane (30 ml) and the reaction mixture stirred for 30 minutes at room temperature under nitrogen. Sodium cyanoborodeuteride (1.46 g, 22.1 mmoles) was then added and the reaction mixture was stirred for 4 days at room temperature. The reaction mixture was quenched with water and extracted with DCM (3×30 mL). The organic solvents were dried over MgSO4 and concentrated to dryness under reduced pressure and the residue purified by flash chromatography over silica gel (0-60% ethyl acetate in hexanes) to give the product as a white solid (1.25 g) LC/MS: 347.1 [M+1]⁺. 1H NMR (300 MHz, CDCl3): δ 7.99 (dd, J=8.1 and 1.8 Hz, 2H), 7.47 (d, J=9.0 Hz, 1H), 7.38 (d, J=8.4 Hz, 2H), 6.70 (dd, J=3.0 and 8.4 Hz, 1H), 6.49 (d, J=2.4 Hz, 1H), 3.92 (s, 3H), 3.73 (s, 3H), 3.56 (s, 1H), 3.36 (t, J=5.4 Hz, 2H), 2.73 (t, J=4.8 Hz, 2H).

### Step 3: 4-((5,5-Dideutero-7-methoxy-2,3-dihydrobenzo[f][1,4]thiazepin-4(5H)-yl)deuteromethyl)benzoic acid

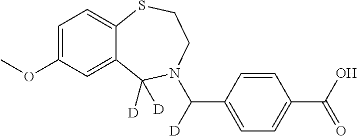 [SMILES: [2H]C(c1ccc(C(=O)O)cc1)N1CCSc2ccc(OC)cc2C1([2H])[2H]]

A round bottom flask was charged with methyl 4-(1-deutero(7-methoxy-5-diduetero-2,3-dihydrobenzo[f][1,4]thiazepin-4(5H)-yl)methyl)benzoate (1.250 g, 3.4 mmol, THF (10 mL), methanol (10 mL), 0.05 mL of water and lithium hydroxide (0.325 g, 13.6 mmol,) and stirred at room temperature overnight. The solvents were concentrated to dryness under reduced pressure and the residue was taken in water and neutralized to pH 7 with 6N HCl. This was extracted with a CHCl₃/IPA mixture (3:1, 3×100 mL). The organic solvents were washed with water, brine and dried over MgSO4. The organic solvents were concentrated to dryness under reduced pressure to give the product as a white solid. LC/MS: 333.2 [M+1]⁺. 1H NMR (300 MHz, DMSO-d6): δ 7.88 (d, J=6.9 Hz, 2H), 7.40 (d, J=8.1 Hz, 1H), 7.32 (d, J=8.7 Hz, 2H), 6.75 (dd, J=9.0 and 3.0 Hz, 1H), 6.62 (d, J=2.4 Hz, 1H), 3.68 (s, 3H), 3.56 (s, 1H), 3.17-3.15 (m, 2H), 2.71-2.68 (m, 2H).

### Example 2

### 4-((7-Methoxy-2,3-dihydropyrido[4,3-f][1,4]thiazepin-4(5H)-yl)methyl)benzoic acid

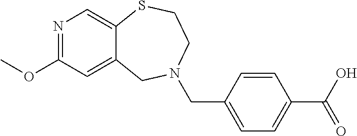 [SMILES: COc1cc2c(cn1)SCCN(Cc1ccc(C(=O)O)cc1)C2]

### Step 1: Methyl 5-((2-((tert-butoxycarbonyl)amino)ethyl)thio)-2-methoxyisonicotinate

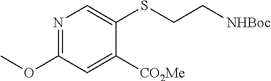 [SMILES: COC(=O)c1cc(OC)ncc1SCCNC(=O)OC(C)(C)C]

A pressure tube was charged with methyl 5-iodo-2-methoxyisonicotinate (7.00 g, 23.9 mmoles), copper(I)iodide (0.910 g, 4.8 mmoles), potassium carbonate (6.60 g, 47.8 mmoles), tert-butyl (2-mercaptoethyl)carbamate (4.04 ml, 23.9 mmoles) and DME (15 ml) and the reaction mixture heated to 80° C. for 3 days. The reaction mixture was filtered over celite and the celite pad washed with DCM (500 mL). The solvents were concentrated to dryness under reduced pressure and the residue purified by flash chromatography over silica gel (0-50% ethyl acetate in hexanes) to give the product as a colorless oil (6.7 g, 75.9%). LC/MS: 342.7 [M+1]⁺. 1H NMR (300 MHz, CDCl3): δ 8.31 s, 1H), 7.03 (s, 1H), 3.95 (s, 6H), 3.32-3.27 (m, 2H), 3.01 (t, J=6.0 Hz, 2H), 1.43 9s, 9H).

### Step 2: Methyl 5-((2-aminoethyl)thio)-2-methoxyisonicotinate

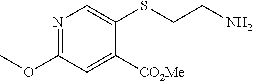 [SMILES: COC(=O)c1cc(OC)ncc1SCCN]

A round bottom flask was charged with methyl 5-((2-((tert-butoxycarbonyl)amino)ethyl)thio)-2-methoxyisonicotinate (6.7 g, 19.6 mmoles), 1,4-dioxan (40 mL) and 4M HCl in 1,4-dioxane (51.06 ml, 204.2 mmoles) was added and the reaction mixture stirred at room temperature overnight under nitrogen. The solvents were concentrated to dryness under reduced pressure to give the product as a white solid (4.4 g). This was used in the next step without further purification. LC/MS: 243.0 [M+1]⁺

### Step 3: 7-Methoxy-3,4-dihydropyrido[4,3-f][1,4]thiazepin-5(2H)-one

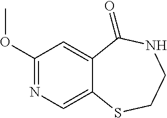

A round bottom flask was charged with methyl 5-((2-aminoethyl)thio)-2-methoxyisonicotinate (4.70 g, 19.4 mmoles), anhydrous THF (20 ml), methanol (20 ml) and sodium methoxide (5.240 g, 97.0 mmoles) was added in one portion at 0° C. The reaction mixture was heated to 48° C. overnight under nitrogen. The reaction mixture was cooled to room temperature and the solvents removed under reduced pressure. The residue was taken in 40 mL of water and extracted with ethyl acetate (3×40 mL). The organic layer was washed with water, brine and dried over MgSO4. The solvents were concentrated to dryness under reduced pressure to dryness to give a tan colored solid (1.7 g, 43.7%). LC/MS: 211.1 [M+1]⁺. 1H NMR (300 MHz, CDCl3): δ 8.26 (s, 1H), 7.04 (s, 1H), 6.8 (bs, 1H), 3.96 (s, 3H), 3.39-3.33 (m, 2H), 3.08 (t, J=6.6 Hz, 2H).

### Step 4: 7-Methoxy-2,3,4,5-tetrahydropyrido[4,3-f][1,4]thiazepine

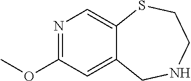

A round bottom flask was charged with lithium aluminum hydride (0.632 g, 16.6 mmoles) and THF (40 mL) and 7-methoxy-3,4-dihydropyrido[4,3-f][1,4]thiazepin-5(2H)-one (1.750 g, 8.3 mmoles) were added under nitrogen and the reaction mixture was heated to 60° C. for 4 h. The reaction mixture was cooled to 0° C. and few drops of water was added and the reaction mixture stirred for 15 minutes. The reaction mixture was then filtered over celite and the filter cake was washed repeatedly with ethyl acetate. The solvents were concentrated to dryness under reduced pressure to give the product as off white solid (1.3 g, 79%). LC/MS: 197.1 [M+1]⁺. 1H NMR (300 MHz, CDCl3): δ 8.27 (s, 1H), 6.60 (s, 1H), 4.04 (s, 2H), 3.91 (s, 3H), 3.41-3.37 (m, 2H), 2.69-2.66 (m, 2H).

### Step 5: Methyl 4-((7-methoxy-2,3-dihydropyrido[4,3-f][1,4]thiazepin-4(5H)-yl)methyl)benzoate

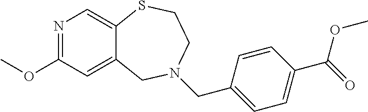

A round bottom flask was charged with 7-methoxy-2,3,4,5-tetrahydropyrido[4,3-f][1,4]thiazepine (0.100 g, 0.5 mmoles), methyl 4-formyl benzoate (0.167 g, 1.0 mmoles) and 1,2-dichloroethane (3 ml) and the reaction mixture stirred for 30 minutes. Sodium cyanoborohydride (0.126 g, 2.0 mmoles) was then added and the reaction mixture was stirred for 3 days. The reaction mixture was quenched with water and extracted with DCM (3×20 ml). The organic layer was washed with water, brine and dried over MgSO4. The solvents were concentrated to dryness under reduced pressure and the residue purified by flash chromatography over silica gel (0-50% ethyl acetate in hexanes) to give the product as a white solid. (73 mg, 41%). LC/MS: 344.1 [M+1]⁺. 1H NMR (300 MHz, CDCl3): δ 8.29 (s, 1H), 7.99 (d, J=8.1 Hz, 2H), 7.35 (d, J=8.4 Hz, 2H), 6.35 (s, 1H), 4.03 (s, 2H), 3.91 (s, 3H), 3.90 (s, 3H), 3.57 (s, 2H), 3.38-3.35 (m, 2H), 2.72-2.69 (m, 2H).

### Step 6: 4-((7-Methoxy-2,3-dihydropyrido[4,3-f][1,4]thiazepin-4(5H)-yl)methyl)benzoic acid

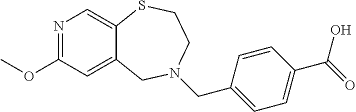 [SMILES: COc1cc2c(cn1)SCCN(Cc1ccc(C(=O)O)cc1)C2]

A round bottom flask was charged with methyl 4-((7-methoxy-2,3-dihydropyrido[4,3-f][1,4]thiazepin-4(5H)-yl)methyl)benzoate (26 mg, 0.1 mmoles), lithium hydroxide (0.007 g, 0.3 mmoles), methanol (3 mL), THF (3 mL) and 0.05 mL of water. The reaction mixture was stirred overnight at room temperature. The organic solvents were removed under reduced pressure and the residue was neutralized with 1N HCl. This was extracted with a CHCl3/IPA mixture (3:1, 3×10 mL). The combined organic layer was washed with water, brine and dried over MgSO4. The organic layer was concentrated to dryness under reduced pressure and the residue purified by flash chromatography over silica gel (0-20% methanol/DCM) to give the product (5 mg, 23%). LC/MS: 331.0 [M+1]⁺. 1H NMR (300 MHz, CD3OD): δ 8.22 (s, 1H), 7.97 (d, J=8.4 Hz, 2H), 7.38 (d, J=8.4 Hz, 2H), 6.44 (s, 1H), 4.07 (s, 2H), 3.91 (s, 3H), 3.87 (s, 3H), 3.36 (s, 2H), 3.34-3.31 (m, 2H), 2.77-2.74 (m, 2H).

### Example 3

### 3-((7-Methoxy-2,3-dihydropyrido[4,3-f][1,4]thiazepin-4(5H)-yl)methyl)benzoic acid

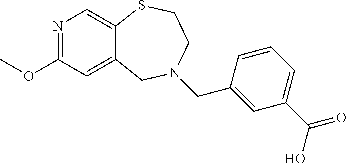 [SMILES: COc1cc2c(cn1)SCCN(Cc1cccc(C(=O)O)c1)C2]

### Step 1: Methyl 3-((7-methoxy-2,3-dihydropyrido[4,3-f][1,4]thiazepin-4(5H)-yl)methyl)benzoate

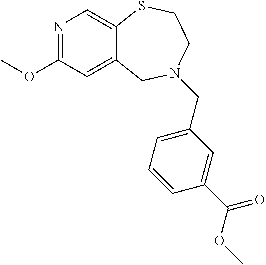 [SMILES: COC(=O)c1cccc(CN2CCSc3cnc(OC)cc3C2)c1]

A round bottom flask was charged with 7-methoxy-2,3,4,5-tetrahydropyrido[4,3-f][1,4]thiazepine (0.100 g, 0.5 mmoles), methyl-3-formylbenzoate (0.167 g, 1.0 mmoles) and 1,2-dichloroethane (3 ml) and the reaction mixture stirred under nitrogen for 30 minutes. Sodium triacetoxyborohydride (0.269 g, 1.3 mmoles) was then added and the reaction mixture was stirred overnight. The reaction mixture was quenched with water and extracted with DCM (3×10 ml). The combined organic solvents were washed with brine and dried over MgSO4. The organic layer was concentrated to dryness under reduced pressure and the residue purified by flash chromatography over silica gel (0-50% ethyl acetate in hexanes) to give the product as colorless oil (115 mg, 65%). LC/MS: 345.0 [M+1]⁺. 1H NMR (300 MHz, CDCl3): δ 8.30 (s, 1H), 7.94 (dd, J=4.8 and 3.0 Hz, 2H), 7.47-7.37 (m, 2H), 6.38 (s, 1H), 4.05 (s, 2H), 3.91 (s, 6H), 3.56 (s, 2H), 3.37-3.34 (m, 2H), 2.72-2.69 (m, 2H).

### Step 2: 3-((7-Methoxy-2,3-dihydropyrido[4,3-f][1,4]thiazepin-4(5H)-yl)methyl)benzoic acid

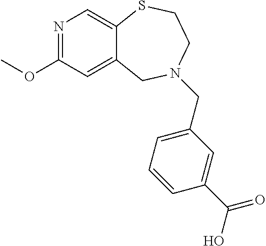 [SMILES: COc1cc2c(cn1)SCCN(Cc1cccc(C(=O)O)c1)C2]

A round bottom flask was charged with methyl 3-((7-methoxy-2,3-dihydropyrido[4,3-f][1,4]thiazepin-4(5H)-yl)methyl)benzoate (115 mg, 0.3 mmoles), MeOH (10 mL), THF (10 ml) and lithium hydroxide (32 mg, 1.3 mmoles) and 0.5 mL of water. The reaction mixture was stirred over night at room temperature. The solvents were removed under reduced pressure and the residue was taken in water and neutralized with 3N HCl. A solid precipitated out which was collected by filtration and washed repeatedly with water and dried to give the product as white solid (62 mg, 54%). LC/MS: 331.1 [M+1]⁺. 1H NMR (300 MHz, DMSO-d6): δ 8.24 (s, 1H), 7.86-7.80 (m, 2H), 7.48-7.39 (m, 2H), 6.57 (s, 1H), 4.02 (s, 2H), 3.81 (s, 3H), 3.55 (s, 2H), 3.21-3.18 (m, 2H), 2.73-2.71 (m, 2H).

### Example 4

### 2-Fluoro-5-((7-methoxy-2,3-dihydropyrido[4,3-f][1,4]thiazepin-4(5H)-yl)methyl)benzoic acid

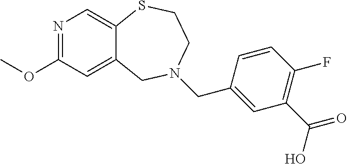 [SMILES: COc1cc2c(cn1)SCCN(Cc1ccc(F)c(C(=O)O)c1)C2]

### Step 1: Methyl 2-fluoro-5-((7-methoxy-2,3-dihydropyrido[4,3-f][1,4]thiazepin-4(5H)-yl)methyl)benzoate

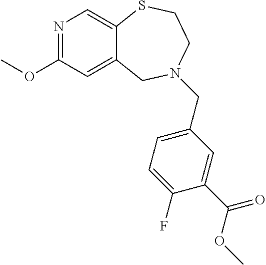 [SMILES: COC(=O)c1cc(CN2CCSc3cnc(OC)cc3C2)ccc1F]

A round bottom flask was charged with 7-methoxy-2,3,4,5-tetrahydropyrido[4,3-f][1,4]thiazepine (0.100 g, 0.5 mmoles), methyl 2-fluoro-5-formylbenzoate (0.186 g, 1.0 mmoles) and 1,2-dichloroethane (3 ml) and the reaction mixture stirred under nitrogen for 30 minutes. Sodium triacetoxy borohydride (0.215 g, 1.0 mmoles) was then added and the reaction mixture was stirred overnight under nitrogen. The reaction mixture was quenched with water and extracted with DCM (2×10 mL). The combined organic solvents were washed with brine and dried over MgSO4. The organic layer was concentrated to dryness under reduced pressure and the residue purified by flash chromatography over silica gel (0-50% ethyl acetate in hexanes) to give the product as white solid (100 mg, 54%). LC/MS: 362.8 [M+1]⁺. 1H NMR (300 MHz, CDCl3): δ 8.29 (s, 1H), 7.82 (dd, J=6.9 and 2.1 Hz, 1H), 7.44-7.39 (m, 1H), 7.13-7.06 (m, 1H), 6.37 (s, 1H), 4.03 (s, 2H), 3.94 (s, 3H), 3.92 (s, 3H), 3.51 (s, 2H), 3.36-3.33 (m, 2H), 2.71-2.68 (m, 2H).

### Step 2: 2-Fluoro-5-((7-methoxy-2,3-dihydropyrido[4,3-f][1,4]thiazepin-4(5H)-yl)methyl)benzoic acid

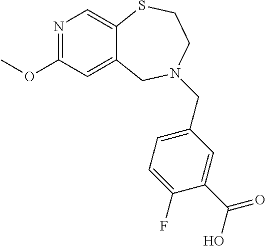 [SMILES: COc1cc2c(cn1)SCCN(Cc1ccc(F)c(C(=O)O)c1)C2]

A round bottom flask was charged with methyl 2-fluoro-5-((7-methoxy-2,3-dihydropyrido[4,3-f][1,4]thiazepin-4(5H)-yl)methyl)benzoate (120 mg, 0.3 mmoles), MeOH (10 mL), THF (10 ml) and lithium hydroxide (32 mg, 1.3 mmoles) and 0.5 mL of water. The reaction mixture was stirred overnight at room temperature. The solvents were removed under reduced pressure and the residue was taken in water and neutralized with 3N HCl and extracted with IPA/CHCl₃ (1:3, 2×15 mL), washed with water, brine and dried over MgSO₄. The organic layer was concentrated to dryness under reduced pressure and the residue dried in a vacuum oven overnight to give the product as white solid (40 mg, 32%). LC/MS: 349.0 [M+1]⁺. 1H NMR (300 MHz, CD₃OD): δ 8.22 (s, 1H), 7.4 (dd, J=7.2 and 2.4 Hz, 1H), 7.53-7.48 (m, 1H), 7.19-7.13 (m, 1H), 6.48 (s, 1H), 4.08 (s, 2H), 3.88 (s, 3H), 3.62 (s, 2H), 3.37-3.34 (m, 2H) 2.79-2.76 (m, 2H).

### Example 5

### 2-((7-Methoxy-2,3-dihydropyrido[4,3-f][1,4]thiazepin-4(5H)-yl)methyl)benzoic acid

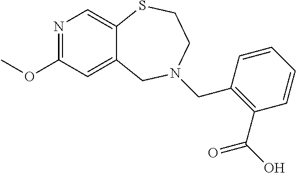 [SMILES: COc1cc2c(cn1)SCCN(Cc1ccccc1C(=O)O)C2]

### Step 1: Methyl 2-((7-methoxy-2,3-dihydropyrido[4,3-f][1,4]thiazepin-4(5H)-yl)methyl)benzoate

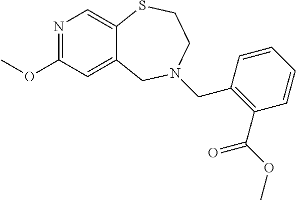 [SMILES: COC(=O)c1ccccc1CN1CCSc2cnc(OC)cc2C1]

A round bottom flask was charged with 7-methoxy-2,3,4,5-tetrahydropyrido[4,3-f][1,4]thiazepine (0.100 g, 0.5 mmoles), methyl 2-formylbenzoate (0.167 g, 1.0 mmoles) and 1,2-dichloroethane (3 ml) and the reaction mixture stirred under nitrogen for 30 minutes. Sodium triacetoxy borohydride (0.215 g, 1.0 mmoles) was then added and the reaction mixture was stirred overnight. The reaction mixture was quenched with water and extracted with DCM (3×10 mL), washed with brine and dried over MgSO4. The organic layer was concentrated to dryness under reduced pressure and the residue purified by flash chromatography over silica gel (0-50% ethyl acetate in hexanes) to give the product as an oil (120 mg, 68%). LC/MS: 345.1 [M+1]⁺. 1H NMR (300 MHz, CDCl3): δ 8.28 (s, 1H), 7.69 (dd, J=7.5 and 1.2 Hz, 1H), 7.40 (dd, J=6.9 and 1.2 Hz, 1H), 7.33-7.27 (m, 2H), 6.40 (s, 1H), 3.95 (s, 2H), 3.91 (s, 3H), 3.82 (s, 3H), 3.77 (s, 2H), 3.30-3.27 (m, 2H), 2.70-2.67 (m, 2H).

### Step 2: 2-((7-Methoxy-2,3-dihydropyrido[4,3-f][1,4]thiazepin-4(5H)-yl)methyl)benzoic acid

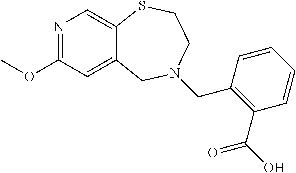 [SMILES: COc1cc2c(cn1)SCCN(Cc1ccccc1C(=O)O)C2]

A round bottom flask was charged with methyl 2-((7-methoxy-2,3-dihydropyrido[4,3-f][1,4]thiazepin-4(5H)-yl)methyl)benzoate (100 mg, 0.3 mmoles), MeOH (10 mL), THF (10 ml) and lithium hydroxide (28 mg, 1.2 mmoles) and 0.5 mL of water. The reaction mixture was stirred overnight at room temperature. The solvents were removed under reduced pressure and the residue was taken in water and neutralized with 3N HCl. This was extracted with IPA/CHCl3 (1:3, 3×10 mL). The combined organic solvents were washed with water, brine and dried over MgSO4. The solvents were concentrated to dryness under reduced pressure and the residue purified by flash chromatography over silica gel (0-20% methanol in DCM) to give the product as a white solid (46 mg, 47%). LC/MS: 331.1 [M+1]⁺. 1H NMR (300 MHz, CD3OD): δ 8.31 (s, 1H), 7.97-7.94 (m, 1H), 7.51-7.48 (m, 2H), 7.35-7.32 (m, 1H), 6.61 (s, 1H), 4.39 (s, 2H), 4.15 (s, 2H), 3.90 (s, 3H), 3.51-3.47 (m, 2H), 3.00-2.97 (m, 2H).

### Example 6

### 2-Methoxy-4-((7-methoxy-2,3-dihydropyrido[4,3-f][1,4]thiazepin-4(5H)-yl)methyl)benzoic acid

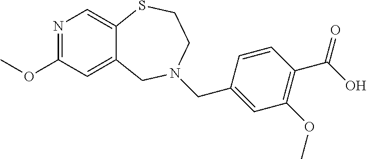 [SMILES: COc1cc2c(cn1)SCCN(Cc1ccc(C(=O)O)c(OC)c1)C2]

### Step 1: Methyl 2-methoxy-4-((7-methoxy-2,3-dihydropyrido[4,3-f][1,4]thiazepin-4(5H)-yl)methyl)benzoate

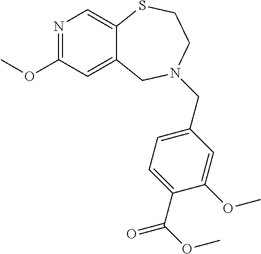

A round bottom flask was charged with 7-methoxy-2,3,4,5-tetrahydropyrido[4,3-f][1,4]thiazepine (0.150 g, 0.8 mmoles), methyl 4-formyl-2-methoxybenzoate (0.297 g, 1.5 mmoles) and 1,2-dichloroethane (3 ml) and the reaction mixture stirred under nitrogen for 30 minutes. Sodium cyanoborohydride (0.189 g, 3.1 mmoles) was then added and the reaction mixture was stirred for 3 days. The reaction mixture was quenched with water and extracted with DCM (2×30 mL). The combined organics were washed with water, brine, dried over MgSO4. The solvents were concentrated to dryness under reduced pressure and the residue purified by flash chromatography over silica gel (0-40% ethyl acetate in hexanes) to give the product as a white solid (100 mg, 35%). LC/MS: 375.0 [M+1]⁺. 1H NMR (300 MHz, CDCl3): δ 8.30 (s, 1H), 7.75 (d, J=8.1 Hz, 1H), 6.93 (s, 1H0, 6.87 (d, J=8.1 Hz, 1H), 6.37 (s, 1H), 4.03 (s, 2H), 3.90 (s, 3H), 3.89 (s, 3H), 3.53 9s, 1H), 3.40-3.37 (m, 2H), 2.72-2.69 (m, 2H).

### Step 2: 2-Methoxy-4-((7-methoxy-2,3-dihydropyrido[4,3-f][1,4]thiazepin-4(5H)-yl)methyl)benzoic acid

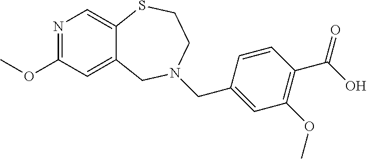 [SMILES: COc1cc2c(cn1)SCCN(Cc1ccc(C(=O)O)c(OC)c1)C2]

A round bottom flask was charged with methyl 2-methoxy-4-((7-methoxy-2,3-dihydropyrido[4,3-f][1,4]thiazepin-4(5H)-yl)methyl)benzoate (100 mg, 0.3 mmoles), MeOH (10 mL), THF (10 ml) and lithium hydroxide (26 mg, 1.1 mmoles) and 0.5 mL of water. The reaction mixture was stirred overnight at room temperature. The reaction was still not complete and so 2 more equivalents of LiOH were added and stirred for another 3 h. The solvents were removed under reduced pressure and the residue was taken in water and neutralized with 3N HCl. A solid precipitated out which was collected by filtration and washed repeatedly with water. The precipitate was then dried in a vacuum to give the product as a white solid (42 mg, 41%). LC/MS: 361.1 [M+1]⁺. 1H NMR (300 MHz, CD3OD): δ 8.23 (s, 1H), 7.79 (d, J=8.1 Hz, 1H), 7.09 (s, 1H), 6.94 (d, J=7.5 Hz, 1H), 6.47 (s, 1H), 4.08 (s, 2H), 3.88 (s, 3H), 3.87 (s, 3H), 3.62 (s, 2H), 3.39-3.36 (m, 2H), 2.79-2.76 (m, 2H).

### Example 7

### 5-((7-Methoxy-2,3-dihydropyrido[4,3-f][1,4]thiazepin-4(5H)-yl)methyl)picolinic acid

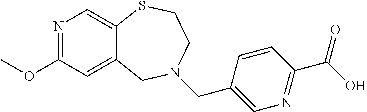 [SMILES: COc1cc2c(cn1)SCCN(Cc1ccc(C(=O)O)nc1)C2]

### Step 1: Methyl 5-((7-methoxy-2,3-dihydropyrido[4,3-f][1,4]thiazepin-4(5H)-yl)methyl)picolinate

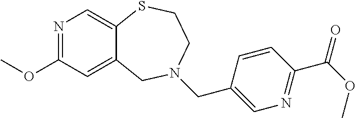 [SMILES: COC(=O)c1ccc(CN2CCSc3cnc(OC)cc3C2)cn1]

A round bottom flask was charged with 7-methoxy-2,3,4,5-tetrahydropyrido[4,3-f][1,4]thiazepine (0.100 g, 0.5 mmoles), methyl 5-formylpicolinate (0.168 g, 1.0 mmoles) and 1,2-dichloroethane (3 ml) and the reaction mixture stirred under nitrogen for 30 minutes. Sodium triacetoxy borohydride (0.215 g, 1.0 mmoles) was then added and the reaction mixture was stirred overnight The reaction mixture was quenched with water and extracted with DCM (2×10 mL). The combined organics were washed with brine and dried over MgSO4. The solvents were concentrated to dryness under reduced pressure and the residue purified by flash chromatography over silica gel (0-100% ethyl acetate in hexanes) to give the product as an oil (70 mg, 40%). LC/MS: 346.0[M+1]⁺. 1H NMR (300 MHz, CDCl3): δ 8.59 (d, J=1.2 Hz, 1H), 8.31 (s, 1H0, 8.12 (d, J=8.1 Hz, 1H), 7.80 (dd, J=8.4 and 2.4 Hz, 1H), 6.35 (s, 1H0, 4.04 (s, 2H0, 4.01 (s, 3H), 3.90 (s, 3H), 3.60 (s, 2H), 3.40-3.37 (m, 2H), 2.72-2.69 (m, 2H).

### Step 2: 5-((7-Methoxy-2,3-dihydropyrido[4,3-f][1,4]thiazepin-4(5H)-yl)methyl)picolinic acid

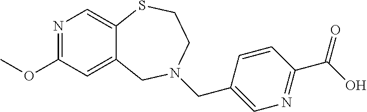 [SMILES: COc1cc2c(cn1)SCCN(Cc1ccc(C(=O)O)nc1)C2]

A round bottom flask was charged with methyl 5-((7-methoxy-2,3-dihydropyrido[4,3-f][1,4]thiazepin-4(5H)-yl)methyl)picolinate (70 mg, 0.2 mmoles), MeOH (10 mL), THF (10 ml) and lithium hydroxide (19 mg, 0.8 mmoles) and 0.5 mL of water. The reaction mixture was stirred overnight at room temperature. The solvents were removed under reduced pressure and the residue was taken in water and neutralized with 3N HCl. A solid precipitated out which was collected by filtration and washed repeatedly with water. This was purified by flash chromatography over silica gel (0-15% methanol in DCM) to give the product as white solid (32 mg, 45%). LC/MS: 332.0 [M+1]⁺. 1H NMR (300 MHz, CD3OD): δ 8.35 (m, 1H), 8.21 (s, 1H), 8.10 (m, 1H), 7.91 (m, 1H), 6.33 (s, 1H), 4.04 (s, 2H0, 4.01 (s, 2H), 3.85 (s, 3H), 3.62 (s, 2H), 3.25 (m, 2H), 2.71 (m, 2H).

### Example 8

### 2-Fluoro-4-((7-methoxy-2,3-dihydropyrido[4,3-f][1,4]thiazepin-4(5H)-yl)methyl)benzoic acid

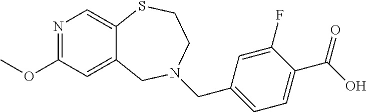 [SMILES: COc1cc2c(cn1)SCCN(Cc1ccc(C(=O)O)c(F)c1)C2]

### Step 1: Methyl 2-fluoro-4-((7-methoxy-2,3-dihydropyrido[4,3-f][1,4]thiazepin-4(5H)-yl)methyl)benzoate

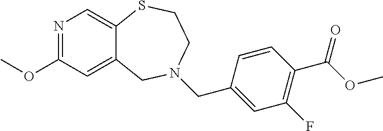

A round bottom flask was charged with 7-methoxy-2,3,4,5-tetrahydropyrido[4,3-f][1,4]thiazepine (0.070 g, 0.4 mmoles), methyl 2-fluoro-4-formylbenzoate (0.097 g, 0.5 mmoles) and 1,2-dichloroethane (3 ml) and the reaction mixture stirred for 30 minutes at room temperature under nitrogen. Sodium triacetoxy borohydride (0.188 g, 0.9 mmoles) was then added and the reaction mixture was stirred overnight at room temperature under nitrogen. The reaction mixture was quenched with water and extracted with DCM (3×10 mL). The combined organics were washed with brine and dried over MgSO4. The organic solvents were concentrated to dryness under reduced pressure and the residue purified by flash chromatography over silica gel (0-50% ethyl acetate in hexanes) to give the product as an oil (80 mg, 62%). LC/MS: 363.2[M+1]⁺. 1H NMR (300 MHz, CDCl3): δ 8.30 (s, 1H), 7.89 (t, J=7.8 Hz, 1H), 7.10 (t, J=11.1 Hz, 2H), 6.36 (s, 1H), 4.04 (s, 2H), 3.93 (s, 3H), 3.91 (s, 3H), 3.54 (s, 2H), 3.38-3.35 (m, 2H), 2.71-2.68 (m, 2H).

### Step 2: 2-Fluoro-4-((7-methoxy-2,3-dihydropyrido[4,3-f][1,4]thiazepin-4(5H)-yl)methyl)benzoic acid

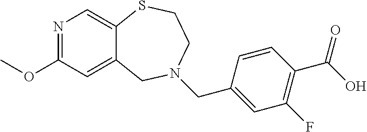 [SMILES: COc1cc2c(cn1)SCCN(Cc1ccc(C(=O)O)c(F)c1)C2]

A round bottom flask was charged with methyl 2-fluoro-4-((7-methoxy-2,3-dihydropyrido[4,3-f][1,4]thiazepin-4(5H)-yl)methyl)benzoate (80 mg, 0.2 mmoles), lithium hydroxide (0.021 g, 0.9 mmoles), methanol (3 mL), THF (3 mL) and 0.1 mL of water. The reaction mixture was stirred over night at room temperature. The organic solvents were removed under reduced pressure and the residue was neutralized with 3N HCl. This was extracted with CHCl₃/IPA mixture (3:1, 3×20 ml). The combined organics were washed with water, brine and dried over MgSO4. The organic solvents were concentrated to dryness under reduced pressure and the residue recrystallized from DCM to give the product as a white solid (40 mg, 48%). LC/MS: 349.0 [M+1]⁺. 1H NMR (300 MHz, CD3OD): δ 8.37 (s, 1H), 8.08 (m, 2H), 7.51-7.48 (m, 2H), 6.97 (s, 1H), 4.73 (s, 2H), 4.53 (s, 2H), 3.95 (s, 3H), 3.70-3.65 (m, 2H), 3.16 (m, 2H).

### Example 9

### 4-((7-Methoxy-2,3-dihydropyrido[2,3-f][1,4]thiazepin-4(5H)-yl)methyl)benzoic acid

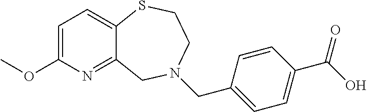 [SMILES: COc1ccc2c(n1)CN(Cc1ccc(C(=O)O)cc1)CCS2]

### Step 1: 3-((2-((tert-Butoxycarbonyl)amino)ethyl)thio)-6-methoxypicolinic acid

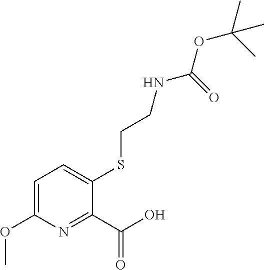 [SMILES: COc1ccc(SCCNC(=O)OC(C)(C)C)c(C(=O)O)n1]

A pressure tube was charged with methyl 3-bromo-6-methoxypicolinate (2.00 g, 8.1 mmoles), potassium carbonate (4.77 g, 34.5 mmoles), tert-butyl (2-mercaptoethyl)carbamate (5.15 ml, 30.5 mmoles) and DMSO (20 mL) and the reaction mixture heated to 60° C. for 3 h in the sealed tube. The reaction mixture was cooled to room temperature and quenched with water and extracted with ethyl acetate (2×40 mL), washed with brine and dried over MgSO4. The organic solvents were concentrated to dryness under reduced pressure to give a white solid (2 g). This was taken in methanol (30 mL), THF (30 mL) and 3 mL of 8N NaOH was added and the reaction mixture was stirred at room temperature overnight. The solvents were removed under reduced pressure and the residue was taken in water (20 mL) and extracted with ethyl acetate (2×60 mL). The organic layer contained only impurity and was discarded. The aqueous layer was neutralized with 3N HCl and extracted with IPA/CHCl3 (1:3, 3×100 mL), washed with water, brine and then dried over MgSO4. The solvents were concentrated to dryness under reduced pressure to give the desired compound as an oil (450 mg, 6.7%). LC/MS: 328.8 [M]⁺1H NMR (300 MHz, CDCl3): δ 7.90 (d, J=8.4 Hz, 1H), 7.05 (d, J=9.0 Hz, 1H), 4.98 (bs, 1H), 3.98 9s, 3H0, 3.43-3.37 (m, 2H0, 3.12-3.08 (m, 2H), 1.44 (s, 9H).

### Step 2: Methyl 3-((2-aminoethyl)thio)-6-methoxypicolinate

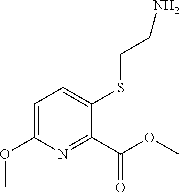 [SMILES: COC(=O)c1nc(OC)ccc1SCCN]

A round bottom flask was charged with 3-((2-((tert-butoxycarbonyl)amino)ethyl)thio)-6-methoxypicolinic acid (0.450 g, 1.4 mmoles), anhydrous DCM (10 mL) under nitrogen at 0° C. Oxalyl chloride (0.353 ml, 4.1 mmoles) was then added slowly followed by 0.05 mL of DMF. The reaction mixture was stirred at room temperature for 2 h. The solvents were removed under reduced pressure to give the product as its HCl salt. This was used directly in the next reaction. LC/MS: 242.9 [M+1]⁺.

### Step 3: 7-Methoxy-3,4-dihydropyrido[2,3-f][1,4]thiazepin-5(2H)-one

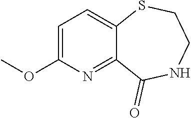

A round bottom flask was charged with methyl 3-((2-aminoethyl)thio)-6-methoxypicolinate (0.300 g, 1.2 mmoles), anhydrous THF (20 ml), methanol (20 ml) and sodium methoxide (0.334 g, 6.2 mmoles) was added in one portion at 0° C. The reaction mixture was heated to 48° C. overnight. LC/MS indicated complete consumption of the starting material with two new peaks, one from the product and another from hydrolysis of the ester. The reaction mixture was cooled to room temperature and the solvents removed under reduced pressure. The residue was taken in 40 mL of water and extracted with ethyl acetate (2×50 mL). The combined organics were washed with brine and dried over MgSO4. Only the desired product came into the organic layer which was concentrated to dryness under reduced pressure to give a tan colored solid (168 mg, 69%). LC/MS: 211.1 [M+1]⁺. 1H NMR (300 MHz, CDCl3): δ 7.67 (d, J=8.1 Hz, 1H), 6.79 (d, J=8.1 Hz, 1H), 4.01 (s, 3H), 3.40-3.35 (m, 2H), 3.13-3.09 (m, 2H).

### Step 4: 7-Methoxy-2,3,4,5-tetrahydropyrido[2,3-f][1,4]thiazepine

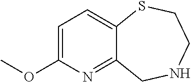

A round bottom flask was charged with 7-methoxy-3,4-dihydropyrido[2,3-f][1,4]thiazepin-5(2H)-one (0.150 g, 0.7 mmoles) and THF (40 mL) and lithium aluminum hydride (0.054 g, 1.4 mmoles) was added and the reaction mixture was heated to 60° C. under nitrogen for 4 h. The reaction mixture was cooled to 0° C. and few drops of water was added and the reaction mixture stirred for 15 minutes. The reaction mixture was then filtered over celite and washed repeatedly with ethyl acetate. The solvents were concentrated to dryness under reduced pressure to give the product as an oil (80 mg, 69%). LC/MS: 197.1 [M+1]⁺. 1H NMR (300 MHz, CDCl3): δ 7.65 (d, J=8.4 Hz, 1H), 6.49 (d, J=8.1 Hz, 1H), 4.18 (s, 2H), 3.96 (s, 3H), 3.37-3.34 (m, 2H), 2.74-2.69 (m, 2H).

### Step 5: Methyl 4-((7-methoxy-2,3-dihydropyrido[2,3-f][1,4]thiazepin-4(5H)-yl)methyl)benzoate

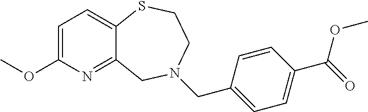

A round bottom flask was charged with 7-methoxy-2,3,4,5-tetrahydropyrido[2,3-f][1,4]thiazepine (0.070 g, 0.4 mmoles), Methyl 4-formyl benzoate (0.100 g, 0.6 mmoles) and 1,2-dichloroethane (3 ml) and the reaction mixture stirred at room temperature under nitrogen for 30 minutes. Sodium triacetoxy borohydride (0.188 g, 0.9 mmoles) was then added and the reaction mixture was stirred overnight. The reaction mixture was quenched with water and extracted with DCM (3×10 mL). The combined organics were washed with brine and dried over MgSO4. The solvents were concentrated to dryness under reduced pressure and the residue purified by flash chromatography over silica gel (0-20% ethyl acetate in hexanes) to give the product as an oil (50 mg, 40%). LC/MS: 345.2[M+1]⁺. 1H NMR (300 MHz, CDCl3): δ 7.98 (dd, J=6.6 and 1.8 Hz, 2H), 7.69 (d, J=9.0 Hz, 1H), 7.40 (d, J=8.4 Hz, 2H), 6.55 (d, J=8.4 Hz, 1H), 4.25 (s, 2H), 3.91 (s, 3H), 3.84 (s, 3H), 3.63 (s, 2H), 3.35-3.31 (m, 2H), 2.75-2.72 (m, 2H).

### Step 6: 4-((7-Methoxy-2,3-dihydropyrido[2,3-f][1,4]thiazepin-4(5H)-yl)methyl)benzoic acid

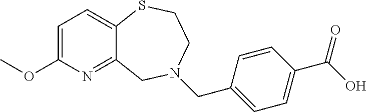 [SMILES: COc1ccc2c(n1)CN(Cc1ccc(C(=O)O)cc1)CCS2]

A round bottom flask was charged with methyl 4-((7-methoxy-2,3-dihydropyrido[2,3-f][1,4]thiazepin-4(5H)-yl)methyl)benzoate (50 mg, 0.1 mmoles), lithium hydroxide (0.014 g, 0.6 mmoles), methanol (3 mL), THF (3 mL) and 0.1 mL of water. The reaction mixture was stirred over night at room temperature. The organic solvents were removed under reduced pressure and the residue was neutralized with 3N HCl. This was extracted with CHCl3/IPA mixture (3:1. 3×10 ml). The combined organics were washed with brine and dried over MgSO4. The solvents were concentrated to dryness under reduced pressure and the residue was recrystallized from DCM to give the product as a pale yellow solid 931 mg). LC/MS: 331.1 [M+1]⁺. 1H NMR (300 MHz, CD3OD): δ 8.15 (d, J=8.1 Hz, 2H), 7.86 (d, J=8.7 Hz, 1H), 7.69 (d, J=8.1 Hz, 2H), 6.84 (d, J=9.0 Hz, 1H), 4.77 (s, 2H), 4.51 (s, 2H), 3.89 (s, 3H), 3.76 (m, 2H), 3.20 (m, 2H).

### Example 10

### 5-((7-Methoxy-2,3-dihydropyrido[2,3-f][1,4]thiazepin-4(5H)-yl)methyl)picolinic acid

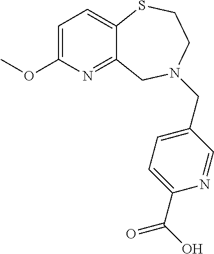 [SMILES: COc1ccc2c(n1)CN(Cc1ccc(C(=O)O)nc1)CCS2]

### Step 1: Methyl 5-((7-methoxy-2,3-dihydropyrido[2,3-f][1,4]thiazepin-4(5H)-yl)methyl)picolinate

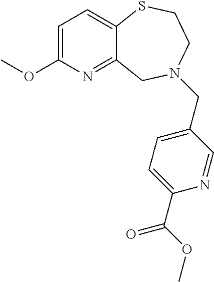 [SMILES: COC(=O)c1ccc(CN2CCSc3ccc(OC)nc3C2)cn1]

A round bottom flask was charged with 7-methoxy-2,3,4,5-tetrahydropyrido[2,3-f][1,4]thiazepine (0.050 g, 0.3 mmoles), methyl 5-formylpicolinate (0.072 g, 0.4 mmoles) and 1,2-dichloroethane (3 ml) and the reaction mixture stirred at room temperature for 30 minutes. Sodium triacetoxy borohydride (0.134 g, 0.6 mmoles) was then added and the reaction mixture was stirred overnight. The reaction mixture was quenched with water and extracted with DCM (3×10 mL). The combined organics were washed with brine and dried over MgSO4. The solvents were concentrated to dryness under reduced pressure and the residue purified by flash chromatography over silica gel (0-60% ethyl acetate in hexanes) to give the product as an oil (32 mg, 34%). LC/MS: 346.0 [M+1]⁺. 1H NMR (300 MHz, CDCL3): δ 8.62 (d, J=1.8 Hz, 1H), 8.10 (d, J=8.4 Hz, 1H), 7.87 (dd, J=7.5 and 1.8 Hz, 1H), 7.70 (d, J=8.7 Hz, 1H), 6.55 (d, J=8.1 Hz, 1H), 4.22 (s, 2H), 4.01 (s, 3H), 3.82 (s, 3H), 3.66 (s, 2H), 3.38-3.35 (m, 2H), 2.76-2.73 (m, 2H).

### Step 2: 5-((7-Methoxy-2,3-dihydropyrido[2,3-f][1,4]thiazepin-4(5H)-yl)methyl)picolinic acid

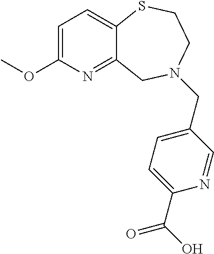 [SMILES: COc1ccc2c(n1)CN(Cc1ccc(C(=O)O)nc1)CCS2]

A round bottom flask was charged with methyl 5-((7-methoxy-2,3-dihydropyrido[2,3-f][1,4]thiazepin-4(5H)-yl)methyl)picolinate (48 mg, 0.1 mmoles), lithium hydroxide (0.014 g, 0.6 mmoles), methanol (3 mL), THF (3 mL) and 0.1 mL of water. The reaction mixture was stirred over night at room temperature. The organic solvents were concentrated under reduced pressure and the residue was neutralized with 3N HCl. This was extracted with CHCl3/IPA mixture (3:1, 3×10 mL). The combined organics were washed with water, brine and dried over MgSO4. The organic solvents were concentrated to dryness and the residue was purified by reverse phase chromatography to give the product (8 mg). LC/MS: 331.9 [M+1]⁺. 1H NMR (300 MHz, CD3OD): δ 8.68 (s, 1H), 8.16 (d, J=16.5 Hz, 2H), 7.79 (d, J=8.1 Hz, 1H), 6.69 (d, J=8.7 Hz, 1H), 4.39 (s, 2H), 4.04 (s, 2H), 3.83 (s, 3H), 3.55 (m, 2H), 2.97 (m, 2H).

### Example 11

### 2-Fluoro-4-((7-methoxy-2,3-dihydropyrido[2,3-f][1,4]thiazepin-4(5H)-yl)methyl)benzoic acid

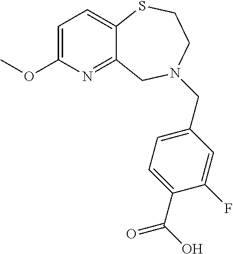 [SMILES: COc1ccc2c(n1)CN(Cc1ccc(C(=O)O)c(F)c1)CCS2]

### Step 1: Methyl 2-fluoro-4-((7-methoxy-2,3-dihydropyrido[2,3-f][1,4]thiazepin-4(5H)-yl)methyl)benzoate

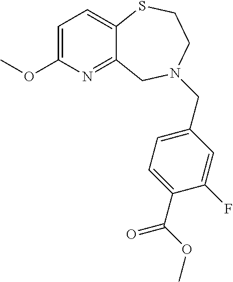 [SMILES: COC(=O)c1ccc(CN2CCSc3ccc(OC)nc3C2)cc1F]

A round bottom flask was charged with 7-methoxy-2,3,4,5-tetrahydropyrido[2,3-f][1,4]thiazepine (0.050 g, 0.3 mmoles), methyl 2-fluoro-4-formylbenzoate (0.079 g, 0.4 mmoles) and 1,2-dichloroethane (3 ml) and the reaction mixture stirred at room temperature for 30 minutes. Sodium triacetoxy borohydride (0.134 g, 0.6 mmoles) was then added and the reaction mixture was stirred overnight. The reaction was complete by LC/MS. The reaction mixture was quenched with water and extracted with DCM (3×10 mL). The combined organic solvents were washed with brine and dried over MgSO4. The solvents were concentrated to dryness under reduced pressure and the residue purified by flash chromatography over silica gel (0-60% ethyl acetate in hexanes) to give the product as an oil (40 mg, 43%). LC/MS: 363.0 [M+1]⁺. 1H NMR (300 MHz, CDCl3): δ 7.87 (t, J=8.4 Hz, 1H), 7.69 (d, J=8.1 Hz, 1H), 7.20-7.13 (m, 2H), 6.55 (d, J=8.4 Hz, 1H), 4.21 (s, 2H), 3.92 (s, 3H), 3.84 (s, 3H), 3.60 (s, 2H), 3.37-3.34 (m, 2H), 2.75-2.72 (m, 2H).

### Step 2: 2-Fluoro-4-((7-methoxy-2,3-dihydropyrido[2,3-f][1,4]thiazepin-4(5H)-yl)methyl)benzoic acid

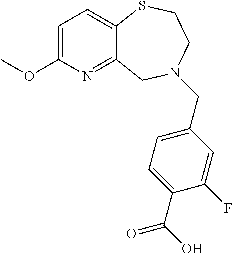 [SMILES: COc1ccc2c(n1)CN(Cc1ccc(C(=O)O)c(F)c1)CCS2]

A round bottom flask was charged with methyl 2-fluoro-4-((7-methoxy-2,3-dihydropyrido[2,3-f][1,4]thiazepin-4(5H)-yl)methyl)benzoate (40 mg, 0.1 mmoles), lithium hydroxide (0.011 g, 0.5 mmoles), methanol (3 mL), THF (3 mL) and 0.1 mL of water. The reaction mixture was stirred over night at room temperature. The organic solvents were removed under reduced pressure and the residue was neutralized with 3N HCl. This was extracted with CHCl3/IPA mixture (3:1, 3×10 mL). The combined organic solvents were washed with brine and dried over MgSO4. The solvents were concentrated to dryness under reduced pressure and the residue was recrystallized from DCM to give the product as a white solid (14 mg). LC/MS: 349.0 [M+1]⁺. 1H NMR (300 MHz, CD3OD): δ 7.88 (t, J=7.8 Hz, 1H), 7.75 (d, J=8.1 Hz, 1H), 7.23 (d, J=8.7 Hz, 2H), 6.62 (d, J=8.1 Hz, 1H), 4.24 (s, 2H), 3.82 (s, 3H), 3.74 (s, 2H), 3.41-3.39 (m, 2H), 2.85-2.82 (m, 2H).

### Example 12

### 2-Methoxy-4-((7-methoxy-2,3-dihydropyrido[2,3-f][1,4]thiazepin-4(5H)-yl)methyl)benzoic acid

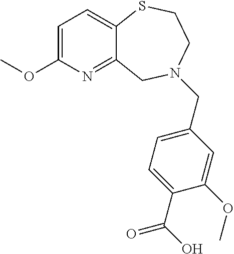 [SMILES: COc1ccc2c(n1)CN(Cc1ccc(C(=O)O)c(OC)c1)CCS2]

### Step 1: Methyl 2-methoxy-4-((7-methoxy-2,3-dihydropyrido[2,3-f][1,4]thiazepin-4(5H)-yl)methyl)benzoate

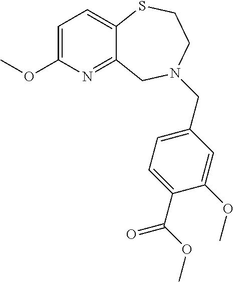 [SMILES: COC(=O)c1ccc(CN2CCSc3ccc(OC)nc3C2)cc1OC]

A round bottom flask was charged with 7-methoxy-2,3,4,5-tetrahydropyrido[2,3-f][1,4]thiazepine (0.050 g, 0.3 mmoles), methyl 4-formyl-2-methoxybenzoate (0.084 g, 0.4 mmoles) and 1,2-dichloroethane (3 ml) and the reaction mixture stirred at room temperature for 30 minutes. Sodium triacetoxy borohydride (0.134 g, 0.6 mmoles) was then added and the reaction mixture was stirred overnight. The reaction was complete by LC/MS. The reaction mixture was quenched with water and extracted with DCM (3×10 mL). The combined organic solvents were washed with brine and dried over MgSO4. The solvents were concentrated to dryness under reduced pressure and the residue purified by flash chromatography over silica gel (0-20% ethyl acetate in hexanes) to give the product as an oil (40 mg, 42%). LC/MS: 375.0[M+1]⁺. 1H NMR (300 MHz, CDCl3): δ 7.75-7.67 (m, 2H), 6.99 (s, 1H), 6.89 (d, J=7.2 Hz, 1H), 6.55 (d, J=8.4 HZ, 1H), 4.27 (s, 2H), 3.89 (s, 3H), 3.88 (s, 3H), 3.84 (s, 3H), 3.60 (s, 2H), 3.33-3.30 (m, 2H), 2.75-2.71 (m, 2H).

### Step 2: 2-Methoxy-4-((7-methoxy-2,3-dihydropyrido[2,3-f][1,4]thiazepin-4(5H)-yl)methyl)benzoic acid

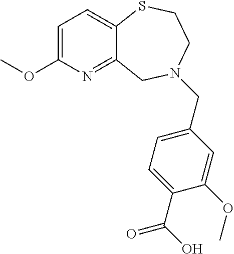 [SMILES: COc1ccc2c(n1)CN(Cc1ccc(C(=O)O)c(OC)c1)CCS2]

A round bottom flask was charged with methyl 2-methoxy-4-((7-methoxy-2,3-dihydropyrido[3,2-f][1,4]thiazepin-4(5H)-yl)methyl)benzoate (40 mg, 0.1 mmoles), lithium hydroxide (0.011 g, 0.5 mmoles), methanol (3 mL), THF (3 mL) and 0.1 mL of water. The reaction mixture was stirred over night at room temperature. The reaction went to completion by LC/MS. The organic solvents were removed under reduced pressure and the residue was neutralized with 3N HCl. This was extracted with CHCl3/IPA mixture (3:1, 3×10 mL). The combined organic solvents were washed with brine and dried over MgSO4. The solvents were concentrated to dryness under reduced pressure and the residue purified by reverse phase HPLC to give the product (8 mg). LC/MS: 361.0 [M+1]⁺. 1H NMR (300 MHz, CD3OD): δ 7.74 (d, J=8.1 Hz, 1H), 7.71 (d, J-8.1 Hz, 1H), 7.11 9s, 1H), 6.95 (d, 6.9 Hz, 1H), 4.23 (s, 2H), 3.86 (s, 3H), 3.82 (s, 3H), 3.66 (s, 2H), 3.33-3.30 (m, 2H), 2.82-2.79 (m, 2H).

### Example 13

### 4-((7-Methoxy-2,3-dihydropyrido[3,2-f][1,4]thiazepin-4(5H)-yl)methyl)benzoic acid

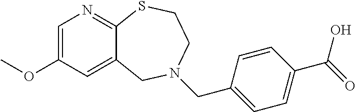 [SMILES: COc1cnc2c(c1)CN(Cc1ccc(C(=O)O)cc1)CCS2]

### Step 1: Methyl 2-chloro-5-methoxynicotinate

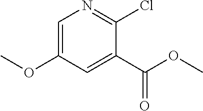

A round bottom flask was charged with 2-chloro-5-methoxynicotinic acid (3.000 g, 16.0 mmoles), methanol (20 ml) and thionyl chloride (5.708 g, 48.0 mmoles) was added at 0° C. under nitrogen and the reaction mixture was stirred for 3 hours at 56° C. The reaction mixture was cooled and the solvents removed under reduced pressure to give the product as a yellow solid. This was used in the next step without further purification. LC/MS: 202.1 (M+1). 1H NMR (300 MHz, CDCL3): δ 8.20 (d, J=2.7 Hz, 1H), 7.67 (d, J=3.0 Hz, 1H), 3.96 (s, 3H), 3.89 (s, 3H).

### Step 2: Methyl 2-((2-((tert-butoxycarbonyl)amino)ethyl)thio)-5-methoxynicotinate

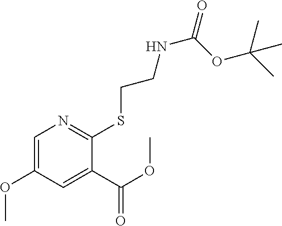 [SMILES: COC(=O)c1cc(OC)cnc1SCCNC(=O)OC(C)(C)C]

A round bottom flask was charged with methyl 2-chloro-5-methoxynicotinate (5.08 g, 25.2 mmoles), DMF (50 mL) and cesium carbonate (16.42 g, 50.4 mmoles) and tert-butyl (2-mercaptoethyl)carbamate (6.39 ml, 37.8 mmoles) under nitrogen and the reaction mixture stirred at room temperature for 2 h. The reaction mixture was quenched with water and extracted with ethyl acetate (3×100 ml). The combined organic solvents were washed with brine and dried over MgSO4. The solvents were concentrated to dryness under reduced pressure and the residue purified (0-30% ethyl acetate in hexanes) to give the product as a colorless oil (2.23 g, 25%). 1H NMR (300 MHz, CDCl₃): δ 8.3 (d, J=3.0 Hz, 1H), 7.76 (d, J=2.4 Hz, 1H), 3.94 (s, 3H), 3.87 (s, 3H), 3.31 (d, J=6.0 Hz, 2H), 2.80 (t, J=6.2 Hz, 2H), 1.43 (s, 9H).

### Step 3: Methyl 2-((2-aminoethyl)thio)-5-methoxynicotinate

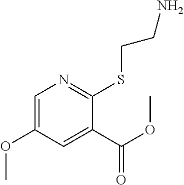 [SMILES: COC(=O)c1cc(OC)cnc1SCCN]

A round bottom flask was charged with methyl 2-((2-((tert-butoxycarbonyl)amino)ethyl)thio)-5-methoxynicotinate (2.23 g, 6.5 mmoles), 1,4-dioxan and 4M HCl in 1,4-dioxan (19.54 ml, 78.2 mmoles) was added and the reaction mixture stirred at room temperature overnight. The solvents were removed under reduced pressure to give the product as a white solid (1.2 g, 76%) which was used in the next step without further purification. LC/MS: 242.9 [M+1]⁺.

### Step 4: 2-((2-Aminoethyl)thio)-5-methoxynicotinic acid

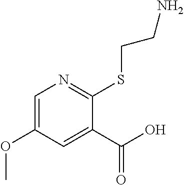

A round bottom flask was charged with methyl 2-((2-aminoethyl)thio)-5-methoxynicotinate (2.02 g, 8.3 mmoles), MeOH (30 mL), THF (30 ml) and lithium hydroxide (1.20 g, 50.1 mmoles) and 0.1 mL of water. The reaction mixture was stirred over night at room temperature. The solvents were removed under reduced pressure and dried in a vacuum oven and used in the next step without further purification. LC/MS: 228.9 [M+1]⁺.

### Step 5: 7-Methoxy-3,4-dihydropyrido[3,2-f][1,4]thiazepin-5(2H)-one

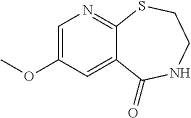

A reaction vial was charged with 2-((2-aminoethyl)thio)-5-methoxynicotinic acid (0.500 g, 2.2 mmoles), DCM (10 mL), EDCI (0.462 g, 2.4 mmoles) and DIPEA (0.572 ml, 3.3 mmoles) and the reaction stirred at room temperature under nitrogen for 2 days. The reaction mixture was filtered and the solvents concentrated to dryness under reduced pressure and the residue purified by flash chromatography to get the product as white solid (80 mg, 17%). LC/MS: 211.1 [M+1]⁺. 1H NMR (3000 MHz, CDCl3): δ 8.33 (d, J=3.3 Hz, 1H), 7.54 (d, J=3.6 Hz, 1H), 6.43 (bs, 1H), 3.89 (s, 3H), 3.50-3.44 (m, 2H), 3.32-3.28 (m, 2H).

### Step 6: 7-Methoxy-2,3,4,5-tetrahydropyrido[3,2-f][1,4]thiazepine

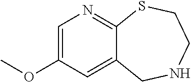

A round bottom flask was charged with lithium aluminum hydride (0.090 g, 2.4 mmoles) and THF (40 mL) and 7-methoxy-3,4-dihydropyrido[3,2-f][1,4]thiazepin-5(2H)-one (0.250 g, 1.2 mmoles) was added and the reaction mixture was heated under nitrogen to 60° C. for 4 h. The reaction mixture was cooled to 0° C. and few drops of water was added and the reaction mixture stirred for 15 minutes. The reaction mixture was then filtered over celite and washed repeatedly with ethyl acetate. The solvents were removed under reduced pressure to give the product as an oil (140 mg). LC/MS: 197.1 [M+1]⁺. 1H NMR (300 MHz, CDCl3): δ 8.05 (d, J=3.0 Hz, 1H), 7.03 (d, J=2.7 Hz, 1H), 4.05 (s, 2H), 3.85 (s, 3H), 3.39-3.36 (m, 2H), 2.85-2.82 (m, 2H).

### Step 7: Methyl 4-((7-methoxy-2,3-dihydropyrido[3,2-f][1,4]thiazepin-4(5H)-yl)methyl)benzoate

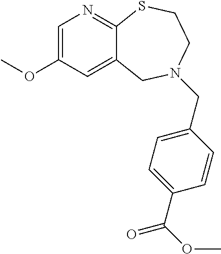

A round bottom flask was charged with 7-methoxy-2,3,4,5-tetrahydropyrido[3,2-f][1,4]thiazepine (0.070 g, 0.4 mmoles), methyl 4-formyl benzoate (0.088 g, 0.5 mmoles) and 1,2-dichloroethane (3 ml) and the reaction mixture stirred under nitrogen for 30 minutes. Sodium triacetoxy borohydride (0.188 g, 0.9 mmoles) was then added and the reaction mixture was stirred overnight. The reaction mixture was quenched with water and extracted with DCM. The combined organic solvents were washed with brine and dried over MgSO4. The solvents were concentrated to dryness under reduced pressure and the residue purified by flash chromatography over silica gel (0-50% ethyl acetate in hexanes) to give the product as an oil (42 mg, 34%). LC/MS: 343.2[M+1]⁺. 1H NMR (300 MHz, CDCl3): δ 8.07 (d, J=3.0 Hz, 1H), 8.01 (d, J=8.7 Hz, 2H), 7.38 (d, J=8.4 Hz, 2H), 6.73 (d, J=2.7 Hz, 1H), 4.00 (s, 2H), 3.92 (s, 3H) 3.78 (s, 3H), 3.66 (s, 2H), 3.38-3.35 (m, 2H), 2.89-2.85 (m, 2H).

### Step 8: 4-((7-Methoxy-2,3-dihydropyrido[3,2-f][1,4]thiazepin-4(5H)-yl)methyl)benzoic acid

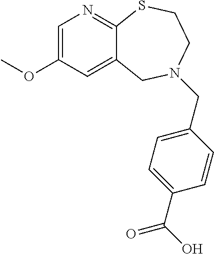 [SMILES: COc1cnc2c(c1)CN(Cc1ccc(C(=O)O)cc1)CCS2]

A round bottom flask was charged with methyl 4-((7-methoxy-2,3-dihydropyrido[3,2-f][1,4]thiazepin-4(5H)-yl)methyl)benzoate (40 mg, 0.1 mmoles), MeOH (10 mL), THF (10 ml) and lithium hydroxide (11 mg, 0.5 mmoles) and few drops of water. The reaction mixture was stirred overnight at room temperature. The solvents were removed under reduced pressure and the residue was taken in water and neutralized with 3N HCl. This was then extracted with IPA/CHCl3 (1:3, 3×10 mL). The combined organic solvents were washed with brine and dried over MgSO4. The solvents were concentrated to dryness under reduced pressure and the product recrystallized from DCM to give the product as white solid (19 mg). LC/MS: 331.0 [M+1]⁺. 1H NMR (300 MHz, CD3OD): δ 8.01-7.99 (m, 3H), 7.45 (d, J=7.5 Hz, 2H), 7.03 (s, 1H), 4.09 (s, 2H), 3.82 (s, 3H), 3.76 (s, 2H), 3.35-3.32 (m, 2H), 2.92-2.89 (m, 2H).

### Example 14

### 2-Methoxy-4-((7-methoxy-2,3-dihydropyrido[3,2-f][1,4]thiazepin-4(5H)-yl)methyl)benzoic acid

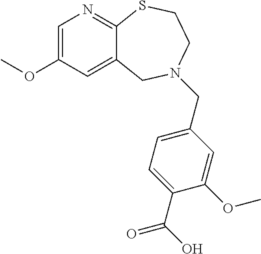

### Step 1: Methyl 2-methoxy-4-((7-methoxy-2,3-dihydropyrido[3,2-f][1,4]thiazepin-4(5H)-yl)methyl)benzoate

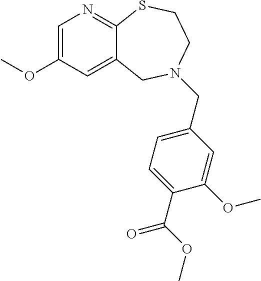

A round bottom flask was charged with 7-methoxy-2,3,4,5-tetrahydropyrido[3,2-f][1,4]thiazepine (0.050 g, 0.3 mmoles), methyl 4-formyl-2-methoxybenzoate (0.084 g, 0.4 mmoles) and 1,2-dichloroethane (3 ml) and the reaction mixture stirred at room temperature for 30 minutes. Sodium triacetoxy borohydride (0.134 g, 0.6 mmoles) was then added and the reaction mixture was stirred overnight. The reaction mixture was quenched with water and extracted with DCM (3×10 ml). The combined organic solvents were washed with brine and dried over MgSO4. The solvents were concentrated to dryness under reduced pressure and the residue purified by flash chromatography over silica gel (0-60% ethyl acetate in hexanes) to give the product as an oil (50 mg, 52%). LC/MS: 375.0 [M+1]⁺. 1H NMR (300 MHz, CDCl3): δ 8.07 (d, J=3.0 Hz, 1H), 7.76 (d, J=8.4 Hz, 1H), 6.99 (s, 1H), 6.87 (d, J=8.1 Hz, 1H), 6.74 (d, J=3.0 Hz, 1H), 4.0 (s, 2H), 3.89 9s, 3H), 3.87 (s, 3H), 3.78 9s, 3H), 3.62 (s, 2H0, 3.39-3.36 (m, 2H), 2.88-2.85 (m, 2H).

### Step 2: 2-Methoxy-4-((7-methoxy-2,3-dihydropyrido[3,2-f][1,4]thiazepin-4(5H)-yl)methyl)benzoic acid

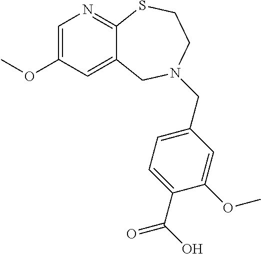

A round bottom flask was charged with methyl 2-methoxy-4-((7-methoxy-2,3-dihydropyrido[3,2-f][1,4]thiazepin-4(5H)-yl)methyl)benzoate (54 mg, 0.1 mmoles), lithium hydroxide (14 mg, 0.6 mmoles), methanol (3 mL), THF (3 mL) and 0.1 mL of water. The reaction mixture was stirred overnight at room temperature. The organic solvents were removed under reduced pressure and the residue was neutralized with 3N HCl. This was extracted with CHCl3/IPA mixture (3:1, 3×10 mL). The combined organic solvents were washed with brine and dried over MgSO4. The solvents were concentrated to dryness under reduced pressure and the residue was purified by reverse phase HPLC (acetonitrile:water) to give the compound as white solid after drying (18 mg). LC/MS: [361.0]⁺. 1H NMR (300 MHz, CD3OD): δ 8.21 (d, J=2.7 Hz, 1H), 7.89 (d, J=7.8 Hz, 1H), 7.42 (d, J=3.0 Hz, 1H), 7.28 (s, 1H), 7.14 (d, J=8.4 Hz, 1H), 4.63 (s, 2H), 4.39 (s, 2H), 3.93 (s, 3H), 3.90 (s, 3H), 3.66-3.63 (m, 2H), 3.23 (m, 2H).

### Example 15

### 2-Fluoro-4-((7-methoxy-2,3-dihydropyrido[3,2-f][1,4]thiazepin-4(5H)-yl)methyl)benzoic acid

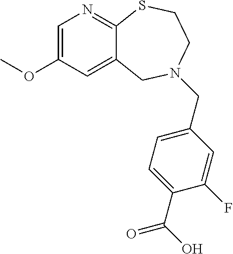 [SMILES: COc1cnc2c(c1)CN(Cc1ccc(C(=O)O)c(F)c1)CCS2]

### Step 1: Methyl 2-fluoro-4-((7-methoxy-2,3-dihydropyrido[3,2-f][1,4]thiazepin-4(5H)-yl)methyl)benzoate

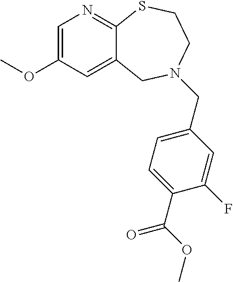 [SMILES: COC(=O)c1ccc(CN2CCSc3ncc(OC)cc3C2)cc1F]

A round bottom flask was charged with 7-methoxy-2,3,4,5-tetrahydropyrido[3,2-f][1,4]thiazepine (0.070 g, 0.4 mmoles), methyl 2-fluoro-4-formylbenzoate (0.097 g, 0.5 mmoles) and 1,2-dichloroethane (3 ml) and the reaction mixture stirred under nitrogen for 30 minutes. Sodium triacetoxy borohydride (0.188 g, 0.9 mmoles) was then added and the reaction mixture was stirred overnight. The reaction was determined complete by LC/MS. The reaction mixture was quenched with water and extracted with DCM (3×10 mL). The combined organic solvents were washed with brine and dried over MgSO4. The solvents were concentrated to dryness under reduced pressure and the residue purified by flash chromatography over silica gel (50% ethyl acetate in hexanes) to give the product as an oil (30 mg, 23%). LC/MS: 363.2[M+1]⁺. 1H NMR (300 MHz, CDCl3): δ 8.07 (d, J=2.4 Hz, 1H), 7.89 (t, J=7.8 Hz, 1H), 7.17-7.12 (m, 2H), 8.75 (d, J=3.0 Hz, 1H), 3.99 (s, 2H), 3.92 (s, 3H), 3.79 (s, 3H), 3.63 (s, 2H), 3.37-3.34 (m, 2H), 2.86-2.83 (m, 2H).

### Step 2: 2-Fluoro-4-((7-methoxy-2,3-dihydropyrido[3,2-f][1,4]thiazepin-4(5H)-yl)methyl)benzoic acid

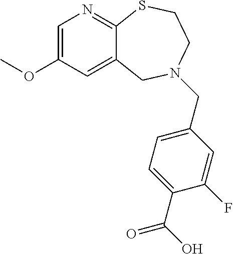 [SMILES: COc1cnc2c(c1)CN(Cc1ccc(C(=O)O)c(F)c1)CCS2]

A round bottom flask was charged with methyl 2-fluoro-4-((7-methoxy-2,3-dihydropyrido[3,2-f][1,4]thiazepin-4(5H)-yl)methyl)benzoate (16 mg, 0.044 mmoles), lithium hydroxide (0.004 g, 0.2 mmoles), methanol (3 mL), THF (3 mL) and 0.1 ml of water. The reaction mixture was stirred over night at room temperature. The organic solvents were removed under reduced pressure and the residue was neutralized with 6N HCl. This was extracted with CHCl3/IPA mixture (75:25, 3×15 ml). The combined organics were washed with water, brine and dried over MgSO4. The solvents were concentrated to dryness under reduced pressure and the residue purified by flash chromatography over silica gel (0-20% methanol/DCM) the product eluted at 5% methanol/DCM. The solvents were concentrated to dryness under reduced pressure to give the product (11 mg, 67%). LC/MS: 349.0 [M+1]⁺. 1H NMR (300 MHZ, CD3OD): δ 8.07 (s, 1H), 7.94 (t, J=7.5 Hz, 1H), 7.33-7.25 (m, 3H), 4.30 (s, 2H), 4.00 (s, 2H), 3.79 (s, 3H), 3.43 (m, 2H), 3.02-3.00 (m, 2H).

### Example 16

### 5-((7-Methoxy-2,3-dihydropyrido[3,2-f][1,4]thiazepin-4(5H)-yl)methyl)picolinic acid

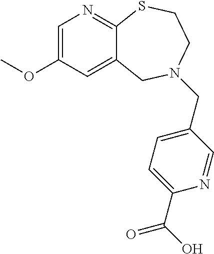 [SMILES: COc1cnc2c(c1)CN(Cc1ccc(C(=O)O)nc1)CCS2]

### Step 1: Methyl 5-((7-methoxy-2,3-dihydropyrido[3,2-f][1,4]thiazepin-4(5H)-yl)methyl)picolinate

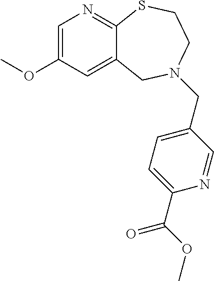 [SMILES: COC(=O)c1ccc(CN2CCSc3ncc(OC)cc3C2)cn1]

A round bottom flask was charged with 7-methoxy-2,3,4,5-tetrahydropyrido[3,2-f][1,4]thiazepine (0.050 g, 0.3 mmoles), methyl 4-formyl-2-methoxybenzoate (0.084 g, 0.4 mmoles) and 1,2-dichloroethane (3 ml) and the reaction mixture stirred at room temperature for 30 minutes. Sodium triacetoxy borohydride (0.134 g, 0.6 mmoles) was then added and the reaction mixture was stirred overnight. The reaction was complete by LC/MS. The reaction mixture was quenched with water and extracted with DCM (3×10 mL). The combined organic solvents were washed with brine and dried over MgSO4. The solvents were concentrated to dryness under reduced pressure and the residue purified by flash chromatography over silica gel (0-60% ethyl acetate in hexanes) to give the product as oil (50 mg, 52%). LC/MS: 375.0 [M+1]⁺. 1H NMR (300 MHz, CDCl₃): δ 8.07 (d, J=3.0 Hz, 1H), 7.76 (d, J=8.4 Hz, 1H), 6.99 (s, 1H), 6.87 (d, J=8.1 Hz, 1H), 6.74 (d, J=3.0 Hz, 1H), 4.0 (s, 2H), 3.89 (s, 3H), 3.87 (s, 3H), 3.78 9s, 3H), 3.62 (s, 2H0, 3.39-3.36 (m, 2H), 2.88-2.85 (m, 2H).

### Step 2: 5-((7-Methoxy-2,3-dihydropyrido[3,2-f][1,4]thiazepin-4(5H)-yl)methyl)picolinic acid

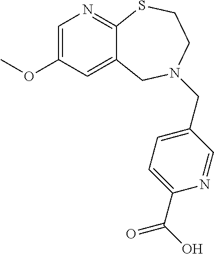 [SMILES: COc1cnc2c(c1)CN(Cc1ccc(C(=O)O)nc1)CCS2]

A round bottom flask was charged with methyl 5-((7-methoxy-2,3-dihydropyrido[3,2-f][1,4]thiazepin-4(5H)-yl)methyl)picolinate (50 mg, 0.1 mmoles), lithium hydroxide (0.014 g, 0.6 mmoles), methanol (3 mL), THF (3 mL) and 0.1 mL of water. The reaction mixture was stirred over night at room temperature. The reaction went to completion by LC/MS. The organic solvents were removed under reduced pressure and the residue was neutralized with 3N HCl. This was extracted with CHCl3/IPA mixture (3:1, 3×10 ml). The combined organic solvents were washed with brine and dried over MgSO4. The solvents were concentrated to dryness under reduced pressure and the residue purified by reverse phase LC/MS: 331.1 [M+1]⁺.

### Example 17

### 4-((2-Methoxy-6,7-dihydropyrimido[4,5-f][1,4]thiazepin-8(9H)-yl)methyl)benzoic acid

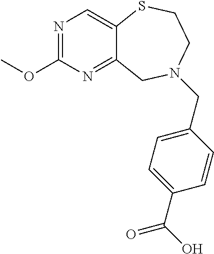 [SMILES: COc1ncc2c(n1)CN(Cc1ccc(C(=O)O)cc1)CCS2]

### Step 1: Ethyl 5-iodo-2-methoxypyrimidine-4-carboxylate

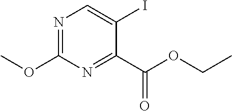

A round bottom flask was charged with ethylpyruvate (7.06 ml, 63.6 mmoles) and the flask was cooled to −10° C. and AcOH (25 mL) was added while maintaining the temperature below −5° C. 30.0% hydrogen peroxide (7.21 ml, 63.6 mmoles) was slowly added dropwise so as to maintain the temperature at −5° C. Another flask was charged with 5-iodo-2-methoxypyrimidine (5.00 g, 21.2 mmoles), toluene (100 ml) and water (25 ml) and the reaction mixture cooled to −10° C. and sulfuric acid (3.388 ml, 63.6 mmoles) was added followed by iron(II)sulfate heptahydrate (17.96 g, 64.6 mmoles). To this, under vigorous stirring, was added the peroxide solution over 1 h while keeping the temperature at −10° C. The reaction mixture was further stirred for 30 minutes. The reaction mixture was poured into ice water and neutralized to pH 7 with 1N NaOH solution and filtered over celite. The celite cake was washed with DCM. The layers were separated and the aqueous layer was extracted with DCM (3×200 mL). The combined organic layers were washed with aq. NaHSO3, brine and dried over Na2SO4. The solvents were concentrated to dryness under reduced pressure and the residue purified by flash chromatography over silica gel (1-50% ethyl acetate in hexanes) to give the product as a colorless oil (1.35 g, 21%). LC/MS: 308.0[M+]+. 1H NMR (300 MHz, CDCl3): δ 8.78 (s, 1H), 4.39 (q, J=7.2 Hz, 2H), 3.95 (s, 3H). 1.36 (t, J=6.9 Hz, 3H).

### Step 2: Ethyl 5-((2-((tert-butoxycarbonyl)amino)ethyl)thio)-2-methoxypyrimidine-4-carboxylate

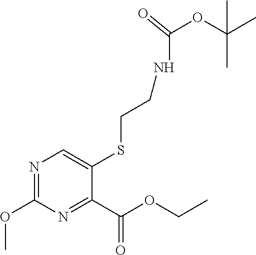 [SMILES: CCOC(=O)c1nc(OC)ncc1SCCNC(=O)OC(C)(C)C]

A pressure tube was charged with ethyl 5-iodo-2-methoxypyrimidine-4-carboxylate (1.900 g, 6.2 mmoles), copper(I)iodide (0.235 g, 1.2 mmoles), potassium carbonate (1.705 g, 12.3 mmoles), tert-butyl (2-mercaptoethyl)carbamate (1.563 ml, 9.3 mmoles) and DME (6 ml) and the sealed reaction reaction tube heated to 80° C. for 3 days. The reaction mixture was filtered over celite and washed with DCM. The filtrate was concentrated to dryness under reduced pressure and the residue purified by flash chromatography over silica gel (0-50% EA in hexanes) to give the product as a colorless oil (1.8 g). LC/MS: 357.6 [M+]⁺ 1H NMR (300 MHz, CDCl3): δ 8.71 (s, 1H), 4.47 (q, J=6.9 Hz, 2H), 4.05 (s, 3H), 3.31-3.28 (m, 2H), 3.01-2.97 (m, 2H), 1.48-1.41 (m, 12H).

### Step 3: Ethyl 5-((2-aminoethyl)thio)-2-methoxypyrimidine-4-carboxylate

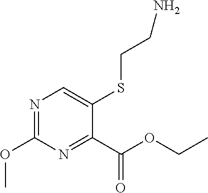 [SMILES: CCOC(=O)c1nc(OC)ncc1SCCN]

A round bottom flask was charged with ethyl 5-((2-((tert-butoxycarbonyl)amino)ethyl)thio)-2-methoxypyrimidine-4-carboxylate (0.600 g, 1.7 mmoles), 1,4-dioxan and TFA (0.386 ml, 5.0 mmoles) was added and the reaction mixture stirred at room temperature overnight. The solvents were concentrated to dryness under reduced pressure to give the product as a yellow oil (430 mg, 99%). This was used in the next step without further purification. LC/MS: 257.0 [M+1]⁺.

### Step 4: 2-Methoxy-7,8-dihydropyrimido[4,5-f][1,4]thiazepin-9(6H)-one

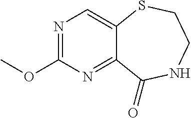

A round bottom flask was charged with ethyl 5-((2-aminoethyl)thio)-2-methoxypyrimidine-4-carboxylate (1.00 g, 3.9 mmoles), anhydrous THF (20 ml), methanol (20 ml) and sodium methoxide (1.05 g, 19.4 mmoles) was added in one portion at 0° C. under nitrogen. The reaction mixture was heated to 48° C. overnight. The reaction mixture was cooled to room temperature and the solvents removed under reduced pressure. The residue was taken in 40 mL of water and extracted with ethyl acetate (2×30 mL). The combined organic solvents were washed with brine and dried over MgSO4. The solvents were concentrated to dryness under reduced pressure to give a tan colored solid (450 mg, 54%). LC/MS: 212.1 [M+1]⁺. 1H NMR (300 MHz, CDCl3): δ 8.62 (s, 1H), 7.01 (bs, 1H), 4.00 (s, 3H), 3.42-3.38 (m, 2H), 3.15-3.11 (m, 2H).

### Step 5: 2-Methoxy-6,7,8,9-tetrahydropyrimido[4,5-f][1,4]thiazepine

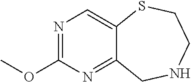

A round bottom flask was charged with lithium aluminum hydride (0.115 g, 3.0 mmoles) and THF (40 mL) and 2-methoxy-7,8-dihydropyrimido[4,5-f][1,4]thiazepin-9(6H)-one (0.320 g, 1.5 mmoles) was added and the reaction mixture was heated to 60° C. under nitrogen for 2 h. The reaction mixture was cooled to 0° C. and few drops of water was added and the reaction mixture stirred for 15 minutes. The reaction mixture was then filtered over celite and washed repeatedly with ethyl acetate. The solvents were removed under reduced pressure to give the product as oil. The crude mixture was used in the following reaction without additional purification. LC/MS: 198.1 [M+1]⁺.

### Step 6: Methyl 4-((2-methoxy-6,7-dihydropyrimido[4,5-f][1,4]thiazepin-8(9H)-yl)methyl)benzoate

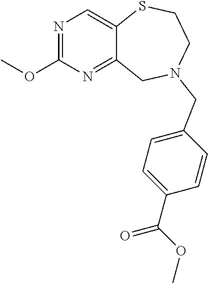 [SMILES: COC(=O)c1ccc(CN2CCSc3cnc(OC)nc3C2)cc1]

A round bottom flask was charged with 2-methoxy-6,7,8,9-tetrahydropyrimido[4,5-f][1,4]thiazepine (0.070 g, 0.4 mmoles), methyl 4-formyl benzoate (0.079 g, 0.5 mmoles) and 1,2-dichloroethane (3 ml) and the reaction mixture stirred at room temperature under nitrogen for 30 minutes. Sodium triacetoxy borohydride (0.188 g, 0.9 mmoles) was then added and the reaction mixture was stirred overnight. The reaction mixture was quenched with water and extracted with DCM (3×10 mL). The combined organic solvents were washed with brine and dried over MgSO4. The solvents were concentrated to dryness under reduced pressure and the residue purified by flash chromatography over silica gel (0-30% ethyl acetate/hexanes) to give the product as an oil (12 mg, 9%). LC/MS: 346.2[M+1]⁺. 1H NMR (300 MHz, CDCl3): δ 8.56 (s, 1H), 7.98 (d, J 8.4 Hz, 2H), 7.35 (d, J=8.4 Hz, 2H), 4.24 (s, 2H), 3.98 (s, 3H), 3.91 (s, 3H), 3.64 (s, 2H), 3.33-3.30 (m, 2H), 2.74-2.70 (m, 2H).

### Step 7: 4-((2-Methoxy-6,7-dihydropyrimido[4,5-f][1,4]thiazepin-8(9H)-yl)methyl)benzoic acid

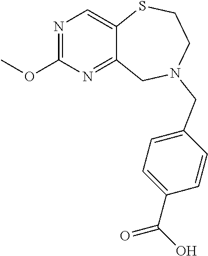 [SMILES: COc1ncc2c(n1)CN(Cc1ccc(C(=O)O)cc1)CCS2]

A round bottom flask was charged with methyl 4-((2-methoxy-6,7-dihydropyrimido[4,5-f][1,4]thiazepin-8(9H)-yl)methyl)benzoate (12 mg, 0.035 mmoles), lithium hydroxide (0.003 g, 0.1 mmoles), methanol (3 mL), THF (3 mL) and 0.05 mL of water. The reaction mixture was stirred over night at room temperature. The organic solvents were removed under reduced pressure and the residue was neutralized with 3N HCl. This was extracted with CHCl3/IPA mixture (3:1, 3×10 ml). The combined organic solvents were washed with brine and dried over MgSO4. The solvents were concentrated to dryness under reduced pressure and the residue recrystallized from DCM to give the product as white solid (4 mg). LC/MS: 332.0 [M+1]⁺. 1H NMR (300 MHz, CD3OD): δ 8.73 (s, 1H), 8.13 (d, J=8.7 Hz, 2H), 7.68 (d, J=8.4 Hz, 2H), 4.71 (s, 2H), 4.49 (s, 2H), 3.79 (s, 3H), 3.76 (t, J=5.4 Hz, 2H), 3.22 (m, 2H). LC/MS: 332.0 [M+1]⁺.

### Example 18

### 2-Fluoro-4-((7-trideuteromethoxy)-2,3-dihydrobenzo[f][1,4]thiazepin-4(5H)-yl)methyl)benzoic acid

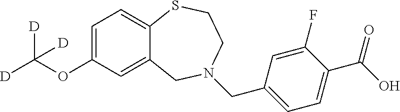 [SMILES: [2H]C([2H])([2H])Oc1ccc2c(c1)CN(Cc1ccc(C(=O)O)c(F)c1)CCS2]

### Step 1: 7-Hydroxy-3,4-dihydrobenzo[f][1,4]thiazepin-5(2H)-one

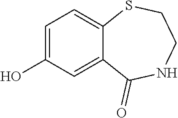 [SMILES: O=C1NCCSc2ccc(O)cc21]

A round bottom flask was charge with 7-methoxy-3,4-dihydrobenzo[f][1,4]thiazepin-5(2H)-one (1.0 g, 4.8 mmoles), anhydrous DCM (60 mL) and the reaction flask was cooled to −78° C. and boron tribromide (4.0 mL, 10.56 mmmols) was added over a period of 10 minutes. The reaction was allowed to warm to room temperature and stirred overnight. The reaction mixture was quenched with methanol and diluted with ice water and extracted with DCM (3×50 mL). The combined organics were washed with water, brine and dried over MgSO4. The solvents were concentrated to dryness under reduced pressure to give the product (0.8 g). LC/MS: 196.0 [M+1]⁺. 1H NMR (300 MHz, DMS)-d6): δ 9.92 (s, 1H), 8.22 (t, J=6.3 Hz, 1H), 7.27 (d, J=8.1 Hz, 1H), 6.91 (d, J=3.0 Hz, 1H), 6.81 (dd, J=8.1 and 3.0 Hz, 1H), 3.15-3.09 (m, 2H), 2.99-2.95 (m, 2H).

### Step 2: 7-(Trideuteromethoxy)-3,4-dihydrobenzo[f][1,4]thiazepin-5(2H)-one

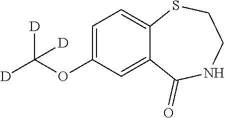 [SMILES: [2H]C([2H])([2H])Oc1ccc2c(c1)C(=O)NCCS2]

A round bottom flask was charged with 7-hydroxy-3,4-dihydrobenzo[f][1,4]thiazepin-5(2H)-one (0.8 g, 4.102 mmols), anhydrous DMF (10 mL), potassium carbonate (2.300 g, 16.408 mmols). The reaction mixture was stirred at room temperature under nitrogen for 30 minutes and iodomethane-d3 (1.014 ml, 16.408 mmol) was added slowly and the reaction mixture stirred for 6 hours. Water was added to quench the reaction and extracted with ethyl acetate (3×50 mL). The combined organics were washed with water, brine and dried over Mg504 to give the product (0.6 g). LC/MS: 213.1 [M+1]⁺. 1H NMR (300 MHz, CDCl3): δ 7.43 (d, J=8.7 Hz, 1H), 7.24 (d, J=3.0 Hz, 1H), 6.94 (dd, J=8.7 and 2.7 Hz, 1H), 3.40-3.34 (m, 2H), 3.13-3.09 (m, 2H).

### Step 3: 7-Trideuteromethoxy-2,3,4,5-tetrahydrobenzo[f][1,4]thiazepine

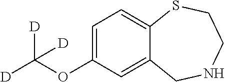 [SMILES: [2H]C([2H])([2H])Oc1ccc2c(c1)CNCCS2]

A round bottom flask was charged with lithium aluminium hydride (0.16 g, 4.245 mmols) and THF (50 ml) was added under nitrogenat 0° C. 7-(Trideuteromeoxy)-3,4-dihydrobenzo[f][1,4]thiazepin-5(2H)-one (0.600 g, 2.83 mmols) was added in portions. The reaction mixture was then refluxed for 6 hours. The reaction mixture was cooled to 0° C. and few drops of water was added and stirred for 15 minutes. This was filtered over celite and washed repeatedly with ethyl acetate. The solvents were concentrated to dryness under reduced pressure to give the product as tan colored solid (0.56 g). LC/MS: 199.0 [M+1]⁺. 1H NMR (300 MHz, CDCl3): δ 7.48 (d, J=8.4 Hz, 1H), 6.80 (d, J=3.0 Hz, 1H), 6.68 (dd, J=8.7 and 2.7 Hz, 1H), 4.11 9s, 2H), 3.41-3.37 (m, 2H), 2.72-2.69 (m, 2H).

### Step 4: Methyl 2-fluoro-4-((7-(trideuteromethoxy)-2,3-dihydrobenzo[f][1,4]thiazepin-4(5H)-yl)methyl)benzoate

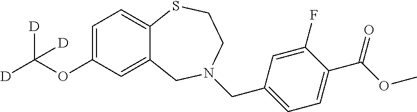 [SMILES: [2H]C([2H])([2H])Oc1ccc2c(c1)CN(Cc1ccc(C(=O)OC)c(F)c1)CCS2]

A round bottom flask was charged 7-trideuteromethoxy-2,3,4,5-tetrahydrobenzo[f][1,4]thiazepine (0.100 g, 0.5 mmoles), 1,2-dichloroethane (5 mL) and methyl 2-fluoro-4-formylbenzoate (0.138 g, 0.8 mmoles). The reaction mixture was stirred under nitrogen for 30 minutes and sodium triacetoxy borohydride (0.266 g, 1.3 mmoles) was added and the reaction mixture stirred at room temperature over night. The reaction was quenched with water and extracted with DCM (3×20 ml). The combined organics were washed with water, brine and dried over MgSO4. The solvents were concentrated to dryness under reduced pressure and the residue purified by flash chromatography over silica gel (0-50% ethyl acetate in hexanes) to give the product as colorless oil (0.13 g, 70%). LC/MS: 365.0 [M+1]⁺. 1H NMR (300 MHz, CDCl3): δ 7.88 (t, J=7.8 Hz, 1H), 7.47 (d, J=9.0 HZ, 1H), 7.17-7.11 (m, 2H), 6.70 (dd, J=8.1 and 2.4 HZ, 1H), 6.49 (d, J=3.0 Hz, 1H), 4.08 (s, 2H), 3.93 (s, 3H), 3.56 (s, 2H), 3.37-3.34 (m, 2H), 2.73-2.70 (m, 2H).

### Step 5: 2-Fluoro-4-((7-trideuteromethoxy)-2,3-dihydrobenzo[f][1,4]thiazepin-4(5H)-yl)methyl)benzoic acid

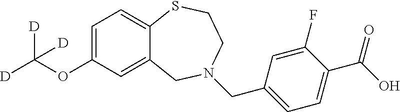 [SMILES: [2H]C([2H])([2H])Oc1ccc2c(c1)CN(Cc1ccc(C(=O)O)c(F)c1)CCS2]

A round bottom flask was charged with methyl 2-fluoro-4-((7-(trideuteromethoxy)-2,3-dihydrobenzo[f][1,4]thiazepin-4(5H)-yl)methyl)benzoate (130 mg, 0.4 mmoles), lithium hydroxide (34 mg, 1.4 mmoles), methanol (5 ml), THF (5 ml) and 0.5 ml of water and the reaction mixture was stirred over night at room temperature. The organic solvents were removed under reduced pressure and the residue was taken in water and neutralized with 3N HCl and extracted with IPA/CHCl3 (1:3, 3×30 mL). The organic solvents were washed with water, brine and dried over MgSO4. The solvents were removed under reduced pressure to give a white solid (115 mg, 91%). LC/MS: 351.0 [M+1]⁺. 1H NMR (300 MHz, CD3OD): δ 7.95 (t, J=7.5 Hz, 1H), 7.50 (d, J=8.1 Hz, 1H), 7.29 (d, J=9.3 Hz, 2H), 6.85 (dd, J=8.7 and 3.0 Hz, 1H), 6.77 (d. J=3.0 Hz, 1H), 4.37 (s, 2H), 4.00 (s, 2H), 3.48-3.45 (m, 2H), 2.92-2.89 (m, 2H).

### Example 19

### 4-(Nitrooxy)butyl 4-((7-methoxy-2,3-dihydrobenzo[f][1,4]thiazepin-4(5H)-yl)methyl)benzoate

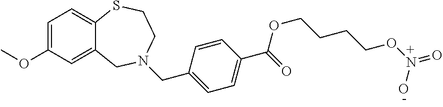

A round bottom flask was charged with 4-hydroxybutyl nitrate (66 mg, 0.5 mmol, 1.0 equiv.), DCM (20 ml) and 4-((7-methoxy-2,3-dihydrobenzo[f][1,4]thiazepin-4(5H)-yl)methyl)benzoic acid (160 mg, 0.5 mmol, 1.0 equiv.) was added followed by N,N′-dicyclohexylcarbodiimide (0.100 g, 0.5 mmol, 1.0 equiv.) and N,N-4-dimethylaminopyridine (0.006 g, 0.05 mmol, 0.1 equiv.) and the reaction mixture was stirred at room temperature under nitrogen overnight. The reaction mixture was filtered and washed with DCM. The solvents were removed under reduced pressure and the residue purified by flash chromatography over silica gel (0-70% ethyl acetate in hexanes) to give the product as pale oil which turned into white solid upon standing in the freezer. LC/MS: 447.0[M+1]⁺. 1H NMR (300 MHz, CDCl3): δ 7.98 (d, J=8.1 Hz, 2H), 7.48 (d, J=8.1 Hz, 1H), 7.39 (d, J=8.4 Hz, 2H), 6.71 (dd, J=8.4 and 3.0 Hz, 1H), 6.51 (d, J=3.0 Hz, 1H), 4.56-4.52 (m, 2H), 4.9-4.36 (m, 2H), 4.09 (s, 2H), 3.74 (s, 3H), 3.59 (s, 2H), 3.37-3.34 (m, 2H0, 2.75-2.71 (m, 2H0, 1.94-1.89 (m, 4H).

### Example 20

### 4-(Nitrooxy)butyl 4-(1-deutero-(7-methoxy-5,5-dideutero-2,3-dihydrobenzo[f][1,4]thiazepin-4(5H)-yl)ethyl)benzoate

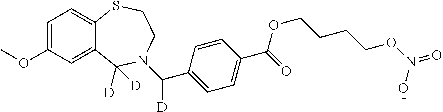

A round bottom flask was charged with 4-hydroxybutyl nitrate (189 mg, 1.4 mmol, 1.0 equiv.), DCM (20 mL) and 4-(1-duetero-(7-methoxy-5-diduetero-2,3-dihydrobenzo[f][1,4]thiazepin-4(5H)-yl)methyl)benzoic acid (460 mg, 1.4 mmol, 1.0 equiv.), N,N′-dicyclohexylcarbodiimide (0.288 g, 1.4 mmol, 1.0 equiv.) and N,N-4-dimethylaminopyridine (0.017 g, 0.14 mmol, 0.1 equiv.) were added and the reaction was stirred under nitrogen overnight. The reaction mixture was filtered and the residue purified by flash chromatography over silica gel (0-70% ethyl acetate in hexanes) to give the product as colorless oil (300 mg). LC/MS: 450.0 (M+1). 1H NMR (300 MHz, CDCl3): δ 7.98 (d, J=8.1 Hz, 2H), 7.47 (d, J=8.4 Hz, 2H), 7.39 9d, J=8.4 Hz, 2H), 6.70 (dd, J=8.4 and 2.4 Hz, 1H), 6.50 (d, J=3.0 Hz, 1H), 4.53 (t, J=6.0 HZ, 2H), 4.37 (t, J=6.7 Hz, 2H), 3.73 (s, 3H), 3.57 9s, 1H), 3.37-3.34 (m, 2H), 2.74-2.71 (m, 2H), 1.93-1.89 (m, 4H).

### Example 21

### 4-(Nitrooxy)butyl 4-((7-methoxy-2,3-dihydropyrido[2,3-f][1,4]thiazepin-4(5H)-yl)methyl)benzoate

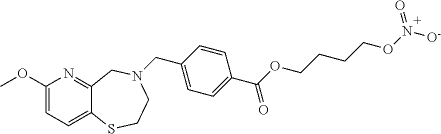

A reaction vial was charged with 4-hydroxybutyl nitrate (0.004 g, 0.03 mmoles), DCM (4 mL) and 4-((7-methoxy-2,3-dihydropyrido[2,3-f][1,4]thiazepin-4(5H)-yl)methyl)benzoic acid (0.010 g, 0.030 mmoles), N,N-dicyclohexylcarbodiimide (0.0061 g, 0.03 mmoles), N,N-dimethylaminopyridine (0.001 g, 0.008 mmoles) were added and the reaction was stirred under nitrogen at room temperature overnight. The reaction mixture was filtered and the filtrate concentrated to dryness and the residue purified by flash chromatography over silica gel (0-60% ethyl acetete in hexanes) to give the product as an oil (6 mg, 44%). LC/MS: 447.9 [M+1]⁺. 1H NMR (300 MHz, CDCl3): δ 7.97 (d, J=8.4 Hz, 2H), 7.69 (d, J=8.7 Hz, 1H), 7.41 (d, J=8.1 Hz, 2H), 6.56 (d, J=8.7 Hz, 1H), 4.53 (t, J=5.7 Hz, 2H), 4.36 (t, J=6.0 Hz, 2H), 4.26 (s, 2H), 3.85 (s, 3H), 3.64 (s, 2H), 3.35-3.32 (m, 2H), 3.74 (m, 2H), 1.93-1.87 (m, 4H).

### Example 22

### 4-(Nitrooxy)butyl 4-((7-methoxy-2,3-dihydropyrido[3,2-f][1,4]thiazepin-4(5H)-yl)methyl)benzoate

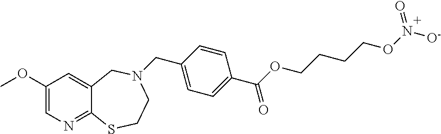

A reaction vial was charged with 4-hydroxybutyl nitrate (0.004 g, 0.03 mmoles), DCM (4 mL) and 4-((7-methoxy-2,3-dihydropyrido[3,2-f][1,4]thiazepin-4(5H)-yl)methyl)benzoic acid (0.011 g, 0.03 mmoles), N,N-dicyclohexylcarbodiimide (0.006 g, 0.03 mmoles), N,N-dimethylaminopyridine (0.001 g, 0.008 mmoles) were added and the reaction was stirred under nitrogen at room temperature overnight. The reaction mixture was filtered and the filtrate concentrated to dryness and the residue purified by flash chromatography over silica gel (0-60% ethyl acetate in hexanes) to give the product as an oil (3 mg, 22%). LC/MS: 447.9 [M+1]⁺. 1H NMR (300 MHz, CDCl3): δ 8.09 (d, J=3.0 Hz, 1H), 8.01 (d, J=8.1 Hz, 2H), 7.42 (d, J=8.1 Hz, 2H), 6.77 (s, 1H), 4.54 (t, 2H), 4.38 (t, 2H0, 4.04 (s, 2H), 3.80 (s, 3H), 3.69 (s, 2H), 3.36-3.35 (m, 2H), 2.89 (m, 2H), 1.94-1.90 (m, 4H).

### Example 23

### 4-(Nitrooxy)butyl 4-((7-methoxy-2,3-dihydropyrido[4,3-f][1,4]thiazepin-4(5H)-yl)methyl)benzoate

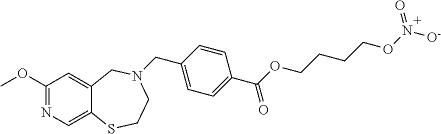

A reaction vial was charged with 4-hydroxybutyl nitrate (0.007 g, 0.052 mmoles), DCM (4 mL) and 4-((7-methoxy-2,3-dihydropyrido[4,3-f][1,4]thiazepin-4(5H)-yl)methyl)benzoic acid (0.017 g, 0.05 mmoles), N,N-dicyclohexylcarbodiimide (0.011 g, 0.05 mmoles), N,N-dimethylaminopyridine (0.001 g, 0.005 mmoles) were added and the reaction was stirred under nitrogen at room temperature overnight. The reaction mixture was filtered and the filtrate concentrated to dryness and the residue purified by flash chromatography over silica gel (0-60% ethyl acetate in hexanes) to give the product as an oil (12 mg, 50%). LC/MS: 447.9 [M+1]⁺. 1H NMR (300 MHz, CD3OD): δ 8.22 (s, 1H), 7.98 (d, J=8.4 Hz, 2H), 87.41 (d, J=8.1 Hz, 2H), 6.44 (s, 1H), 4.57 (t, J=6.0 Hz, 2H), 4.36 (t, J=6.0 Hz, 2H), 4.08 (s, 2H), 3.87 (s, 3H), 3.64 (s, 2H), 3.37-3.34 (m, 2H), 2.78-2.74 (m, 2H), 1.92-1.88 (m, 4H).

### Example 24

### 4-(Nitrooxy)butyl 2-fluoro-4-((7-(trideuteromethoxy)-2,3-dihydrobenzo[f][1,4]thiazepin-4(5H)-yl)methyl)benzoate

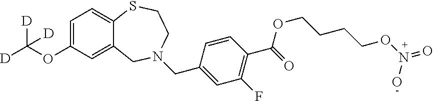

A reaction vial was charged with 4-hydroxybutyl nitrate (0.012 g, 0.08 mmoles), DCM (4 mL) and 2-Fluoro-4-((7-trideuteromethoxy)-2,3-dihydrobenzo[f][1,4]thiazepin-4(5H)-yl)methyl)benzoic acid (0.030 g, 0.08 mmoles), N,N-dicyclohexylcarbodiimide (0.018 g, 0.08 mmoles), N,N-dimethylaminopyridine (0.001 g, 0.008 mmoles) were added and the reaction was stirred under nitrogen at room temperature overnight. The reaction mixture was filtered and the filtrate concentrated to dryness and the residue purified by flash chromatography over silica gel (0-60% ethyl acetate in hexanes) to give the product as an oil (23 mg). LC/MS: 468.0 [M+1]⁺. 1H NMR (300 MHz, CDCl3): δ 7.87 (t, J=7.8 Hz, 1H), 7.48 (d, J=9.0 Hz, 1H), 7.17-7.12 (m, 2H), 6.70 (dd, J 8.4 and 2.4 Hz, 2H), 6.50 (d, J=3.0 Hz, 1H), 4.54 (t, J=5.7 Hz, 2H), 4.38 (t, J=5.7 Hz, 2H), 4.09 (s, 2H), 3.56 (s, 2H), 3.38-3.34 (m, 2H), 2.73-2.70 (m, 2H), 1.93-1.90 (m, 4H).

### Example 25

### 4-(Nitrooxy)butyl 4-((2-methoxy-6,7-dihydropyrimido[4,5-f][1,4]thiazepin-8(9H)-yl)methyl)benzoate

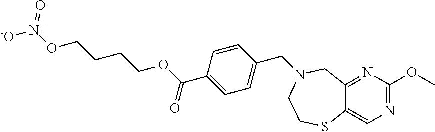

A reaction vial was charged with 4-hydroxybutyl nitrate (0.002 g, 0.015 mmoles), DCM (4 mL) and 4-((2-methoxy-6,7-dihydropyrimido[4,5-f][1,4]thiazepin-8(9H)-yl)methyl)benzoic acid (0.005 g, 0.015 mmoles), N,N-dicyclohexylcarbodiimide (0.003 g, 0.015 mmoles), N,N-dimethylaminopyridine (0.001 g, 0.008 mmoles) were added and the reaction was stirred under nitrogen at room temperature overnight. The reaction mixture was filtered and the filtrate concentrated to dryness and the residue purified by flash chromatography over silica gel (0-60% ethyl acetate in hexanes) to give the product as an oil (2 mg). LC/MS: 448.9 [M+1]⁺. 1H NMR (300 MHz, CDCl3): δ 8.58 (s, 1H), 7.97 (d, J=7.5 Hz, 2H), 7.36 (d, J=8.1 Hz, 2H), 4.53 (t, J=5.7 Hz, 2H), 4.36 (t, 2H), 4.24 (s, 2H), 3.97 (s, 3H), 3.35-3.30 (m, 2H), 2.73-2.70 (m, 2H), 1.92-1.88 (m, 4H).

### Biological Example 1

YS Mouse Model—Flexor Digitorum Brevis (FDB) Muscle Assay (Knoblauch et al., 2013, Lanner et al., 2012).

At least 13 mutations in the skeletal muscle RyR1 are associated with life-threatening responses to exertion, heat challenge and febrile illness. One specific point mutation has been identified in the RyR1 gene in families that exhibit susceptibility to both malignant hyperthermia (MH) and central core disease (CCD). That mutation changes a conserved tyrosine residue at position 522 to a serine residue and is positioned relatively close to five of the six known MH/CCD mutations in the amino terminal region of the RyR1 protein (Quane et al., 1994). A mouse model was created by knocking-in a Y522S (Y524S in mice) mutation in RyR1 associated with MH in humans (Chelu et al., 2006; Durham et al., 2008). The heterozygous mice (RyR1Y524S/WT or YS) demonstrate typical hallmarks of MH (e.g. whole body contractures, elevated core temperature, rhabdomyolysis and death) upon exposure to inhalation anesthetics, and also display an enhanced susceptibility to a heat stroke-like response leading to sudden death when exposed to elevated environmental temperatures (>37° C.) or when exercising under warm (>25° C.) conditions (Chelu et al., 2006). YS-mice are therefore an appropriate and sensitive preclinical model for the study of MH and CCD, as well as being valuable for studying general RyR1-associated disorders as the mutant RyR1 is leaky and calcium handling is altered. Of special interest is the ability of drug substances to alter Ca²⁺ release via RyR1 in isolated FDB fibers from YS mice using fluorescent Ca²⁺ indicators.

### FDB Fiber Isolation:

The FDB muscle was removed and immediately placed into Dulbecco's modified Eagle's medium (DMEM) containing 3 mg/mL collagenase and 10% (v/v) fetal bovine serum. After a two-hour incubation at 37° C., whole FDB muscles were transferred to 1 mL of DMEM and plunged ten times through a 1 mL pipette tip to separate individual fibers. Next, 150 μL of DMEM containing separated FDB fibers were placed onto a 25 mm glass coverslip that had been incubated for 2 hours with 20 μg/mg of laminin in PBS and then subjected to two washes in PBS and a final wash in DMEM. Prior to use, plated fibers were incubated overnight at 37° C. in DMEM containing antibiotic-antimycotic (Gibco, Carlsbad, Calif., USA).

### Isolated FDB Fiber Preparation and Imaging:

After an overnight incubation, the FDB fibers were further incubated for 1 hour at room temperature in either DMEM containing fura-2-acetoxymethyl ester (Fura-2 AM, 10 μM) or incubated for 30 minutes in DMEM containing Mag-fluo-4 (5 μM), with contraction-inhibitor 4-methyl-N-(phenylmethyl)benzenesulfonamide (BTS, 20 μM). Fibers were placed in a temperature controlled chamber (Dagan Corporation, Minneapolis, Minn., USA) on the stage of an inverted epifluorescence microscope (Nikon Inc, Melville, N.Y., USA) and warmed to 32° C. over a 5-minute period in Tyrode's solution. Fluorescence emission was captured using a high speed, digital QE CCD camera (TILL Photonics, Pleasanton, Calif., USA).

### 4-CMC-Induced Ca²⁺ Store Depletion in Isolated Fibers:

To evaluate the effects of drug substance on SR Ca²⁺ store depletion, isolated fibers were exposed to 4-chloro-m-cresol (4-CmC) after 3 minutes of incubation with the drug substances of the present invention. 4-CmC was applied to YS fibers at a dose of 1 mM and WT fibers at a dose of 2.5 mM.

### Drug Preparation:

The stock solutions of all the example compounds were prepared in DMSO to a concentration of 10 mM. On experimental day, FDB fibers was pretreated with 10 μM compound or equivalent volume of DMSO for 2 or 2.5 hours before incubation with florescence dyes (Fura 2 AM or Mega Fura 4).

### Measurement of Ca² Transients During Repetitive Stimulation:

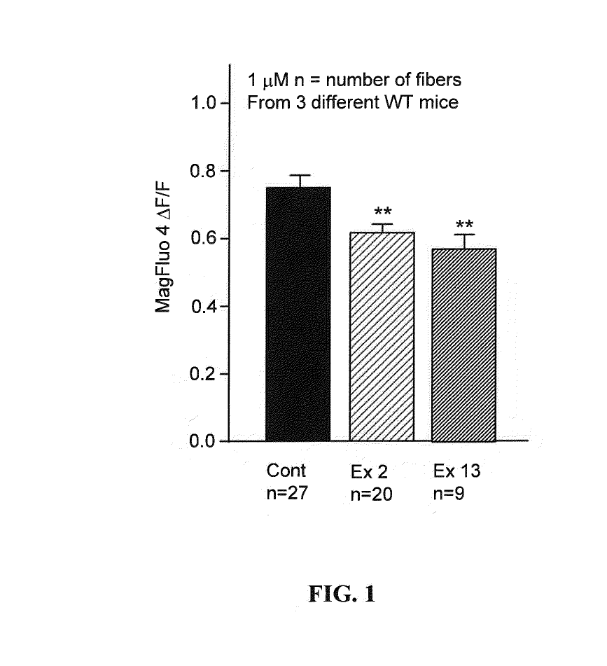

After incubating with drugs (10 μM) for 2.5 hours in the culture medium (DMEM plus 5% FBS and antibiotics), FDB fibers prepared from wild type mice (C57-BLJ) were loaded with 4 μM of mag-fluo-4-AM in fresh DMEM containing 20 μM BTS and 10 μM of each drug or vehicle (DMSO) for 30 minutes at room temperature, followed by two washouts with fresh DMEM. Electrical stimulation was performed using two platinum wires placed at each end of the fiber and uninterrupted electrical trains (100 Hz, 250 ms, every 1.5 seconds; 0.17 duty cycle) was then applied for 300 seconds. To measure RyR1-releasable SR Ca²⁺ store, 1 mM of 4CmC was perfused at 3.25 ml/min immediately after the above mentioned repetitive stimulation. Mag-fluo-4 fluorescence was collected at 20 Hz. Data were collected and analyzed using Metafluor version 6.2 software (Molecular Devices, California, United States). The average of F0/F during the first 10 stimulations was calculated and compared between drug-treated and vehicle-treated groups using un-paired t-test. P<0.05 is considered significantly different. The results for the Example compounds of the present invention are shown in FIG. 1.

### Measurement of Heating-Induced Intracellular Calcium Change:

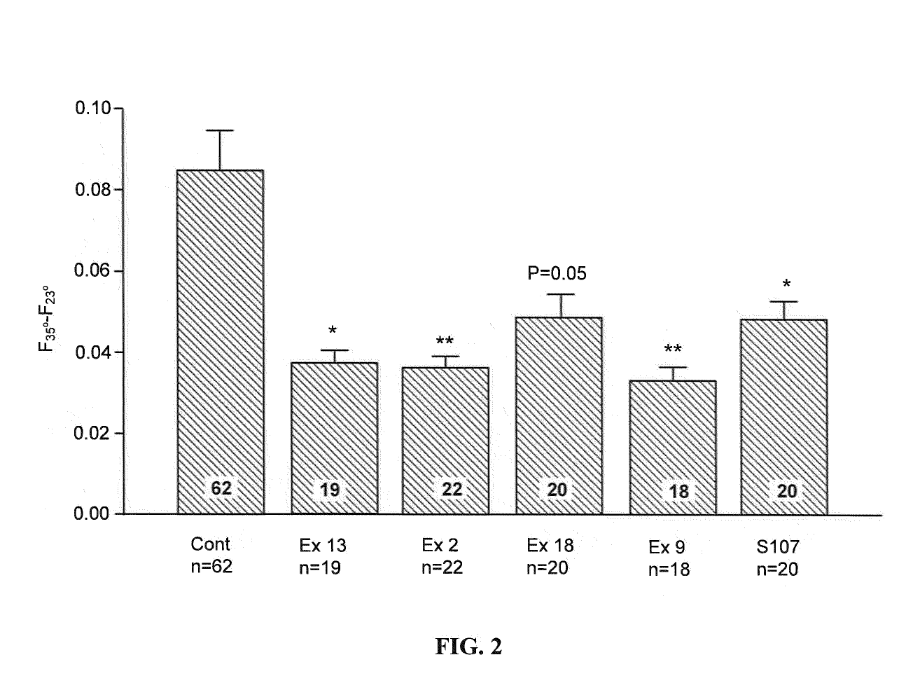

To evaluate the effect of the drugs on heating-induced calcium leak through RyR1, each compound (10 μM) or vehicle was incubated with FDB fibers isolated from YS mice for 2 hours in the culture medium. FDB fibers were mounted to the chamber and loaded with 5 μM Fura 2AM in fresh DMEM containing 20 μM BTS and 10 μM of each drug or vehicle for another 1 hour at room temperature, followed by two washouts with fresh DMEM. Resting fura-2 ratios (R=F340/F380) was recorded for 3 minutes at room temperature and the temperature in the chamber was increased steadily to 32° C. or 35° C. using a SF-28 in-line heater and a bipolar temperature controller TC344B (Warner Instruments). Fura-2 ratios (R=F340/F380) at each temperature were recorded continuously until 2 minutes after reaching plateau. The changes of fluorescence ration (R) from room temperature to 32° C. or 35° C. for each fiber was calculated and then compared between drug-treated and vehicle-treated fibers using unpaired t-test. P<0.05 is considered significantly different. The results for the Example compounds of the present invention along with experimental compound S107 are shown in FIG. 2.

### Statistical Analysis:

A Student's t-test was used for comparison between groups to test significance values of P<0.05 (*), P<0.01 (**), and P<0.001 (***). Dose-response curves were fit using 4-parameter (oxygen consumption (VO2)) or 3-parameter (single-fiber dose-response) Hill function curves in SigmaPlot, version 12.0 (Systat Software, San Jose, Calif., USA). YS data was additionally fitted with a biphasic function using GraphPad Prism, version 6 (GraphPad Software, La Jolla, Calif., USA).

FIG. 1 shows the effects of the test compounds of Example 2 and Example 13, on activity dependent changes in intracellular Ca²⁺ concentrations measured with a Ca²⁺ indicator (MagFluo 4) in FDB fibers from WT mice. The FDB fibers were isolated as described above and after loading with MagFluo 4, the effects of the test compounds on the amplitude of the calcium transients with repetitive electrical stimulation was assessed (FIG. 1). Both Example 2 and Example 13 reduced FDB Ca²⁺ transients>20% at 1 μM.

FIG. 2 shows the effects of Example 2, Example 9, Example 13, Example 18 and experimental compound S107 on heating-induced intracellular calcium change in FDB fibers from YS mice. The FDB fibers were isolated as described and after loading with Fura 2AM, the effects of the test compounds on the heating-induced intracellular calcium change was assessed as described. The experimental compound, S107, has been used by various investigators (Lehnart et al., 2008) over recent years to highlight the potential of this compound to treat conditions associated with aberrant Ca²⁺ handling. For example, compound S107 has been shown to inhibit sarcoplasmic reticulum Ca²⁺ leak, reduce biochemical and histological evidence of muscle damage, improve muscle function and increase exercise performance in mdx mice (Bellinger et al., 2009). It has also been demonstrated that treating sarcoglycan beta deficient mice (Sgcb−/− mice; a murine model for type 2E human limb girdle muscular dystrophy) with the experimental S107 improved muscle specific force, calcium transients, and exercise capacity (Andersson et al., 2012). Treating aged mice with the experimental compound S107 reduced intracellular calcium leak, decreased reactive oxygen species, and enhanced tetanic Ca²⁺ release, muscle-specific force, and improved exercise capacity (Andersson et al., 2011). Experimental compound S107 is therefore a suitable control compound in assays that highlight the calcium modulating activity and therapeutic potential of the compounds of the present invention.

### Biological Example 2

### Analysis of Test Compounds on Calcium Transient Dynamics in Cardiomyocytes Derived from Human Stem Cells by Kinetic Image Cytometry

Culturing of iCell Cardiomyocytes:

Cryopreserved cells (Cellular Dynamics International) were plated onto 96 well tissue culture plates previously coated with Matrigel (250 μg/ml) at a density of 25,000 cells per well, and maintained for two days in plating medium at 37° C. and 7% CO2. After two days in culture, the plating media was replaced with maintenance media and the cells were kept at 37° C. and 7% CO2 with the maintenance media replaced every other day. The cardiomyocytes were maintained in culture for 10 days prior to their use in the assay. The cardiomyocytes displayed a spontaneous beating 48-72 hours after being plated and maintained this phenotype throughout the duration of the experiment.

Loading of Cardiomyocytes with Fluo-4 and Hoechst:

Prior to treatment with the test compounds, the cardiomyocytes were loaded using Fluo-4NW, Hoechst 33342 (200 ng/ml), and 2.5 mM probenecid in the supplied Fluo-4NW assay buffer. Cells were loaded for 1 hour at 37° C.

Exposure of hIPS Cardiomyocytes to Test Compounds:

Test compounds were diluted to their final concentration in Tyrode's solution and brought to 37° C. In addition to standard 2 mM CaCl2 Tyrode's solution, elevated calcium variations using 4 mM and 6 mM CaCl2 were examined in separate test sets. This range of Ca²⁺ concentration promoted prolongation and arrhythmia of the cardiomyocytes (by overload of the sarcoplasmic reticulum calcium stores, leading to store-overload induced calcium release (SOICR) arrhythmia), and demonstrated the ability of the test compounds for proarrhythmia reduction.

Electrical Stimulation of hIPS Cardiomyocytes with Test Compounds:

The hIPS Cardiomyocytes were also prepared for testing with 1 Hz electrical stimulation in standard 2 mM CaCl2 Tyrodes, and test compounds were prepared as described above. Electrical pacing of cardiomyocytes interacts with spontaneous calcium cycling to induce arrhythmia, especially near the onset and end of pacing. Methods of elevated calcium and electrical stimulation have commonly been used for similar arrhythmogenic purposes (Itzahaki et al., 2012; Novak et al., 2012; Hunt et al., 2007).

Treatment with Test Compounds:

Following dye loading, the cells were washed twice with Tyrode's solution, and the test compounds (diluted to their final test concentrations in the appropriate (2 mM, 4 mM or 6 mM CaCl2) Tyrode's solution) were added to the wells. Test compounds were examined at multiple concentrations in triplicate; with the cardiomyocytes incubated with test compounds at 37° C. for 20 minutes prior to being imaged. Bay K8644 (1 uM; a voltage-sensitive L-type dihydropyridine Ca²⁺ channel agonist and positive inotrophic agent), and nifedipine (1 uM; a voltage-sensitive L-type dihydropyridine Ca²⁺ channel blocker and therapeutically used antianginal and antihypertensive agent) were included under each experimental condition as reference compounds and 0.1% DMSO as vehicle control; with Bay K8644 producing a CTD75 prolongation to ≧125% of vehicle control and nifedipine producing a CTD75 shortening to ≦85% of vehicle control.

### Kinetic Imaging of Calcium Transients:

Vala Sciences' Kinetic Image Cytometer (KIC™) was used to capture movies of the intracellular calcium transients. The environmental control chamber of the KIC was set to 37° C. and the KIC was fitted with a 20× objective and captured one image of the nuclei (Hoechst), then recorded 10 seconds of spontaneous activity (Fluo-4NW) at 30 fps from each well.

Transient Analysis with CyteSeer®:

Calcium transient analysis was performed using Vala Sciences' CyteSeer® automated image analysis software. CyteSeer identified, segmented and indexed each cell in each field of view to allow for cell by cell reporting of Ca²⁺ transient measurements. Gating was applied to wells to remove non-responders. All transients recorded in each well were analyzed.

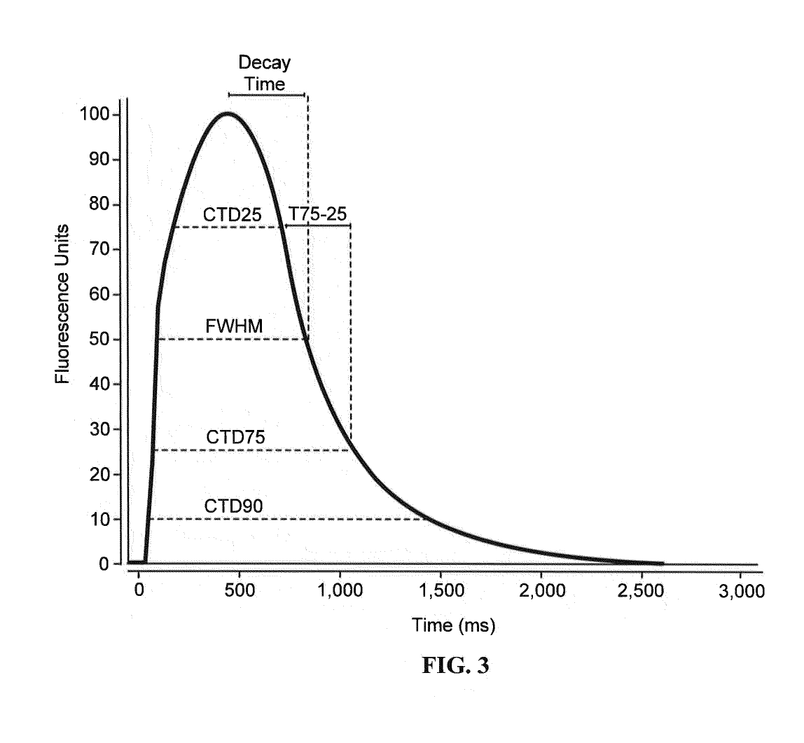

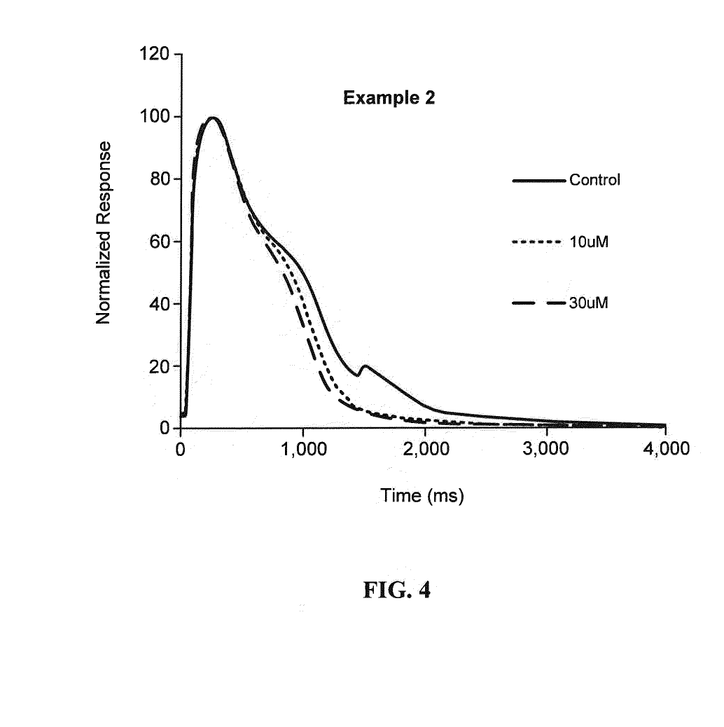

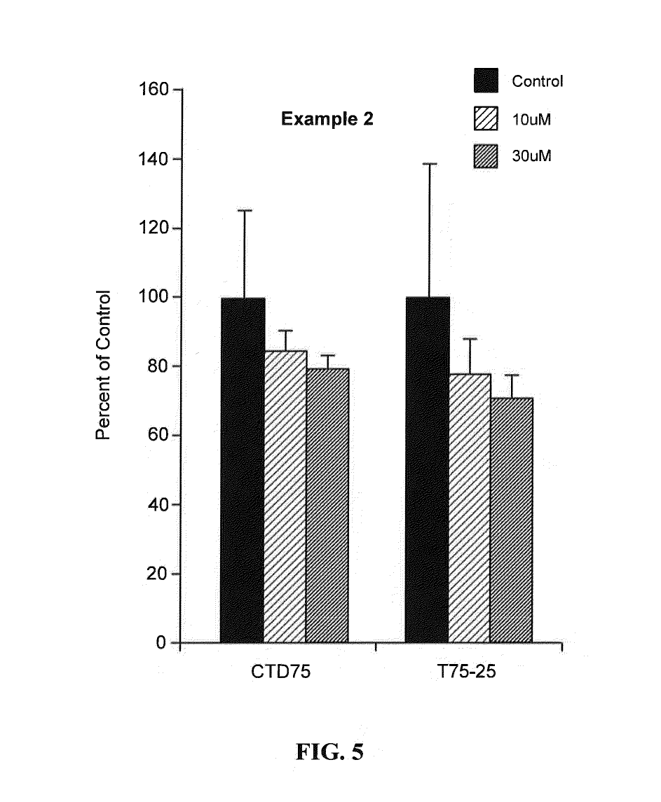

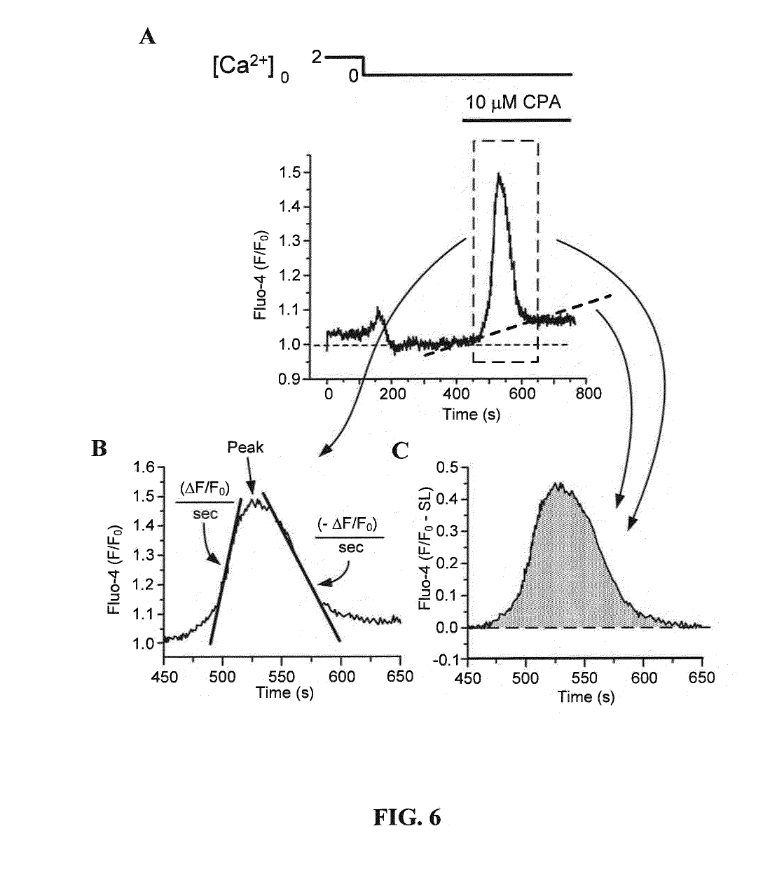

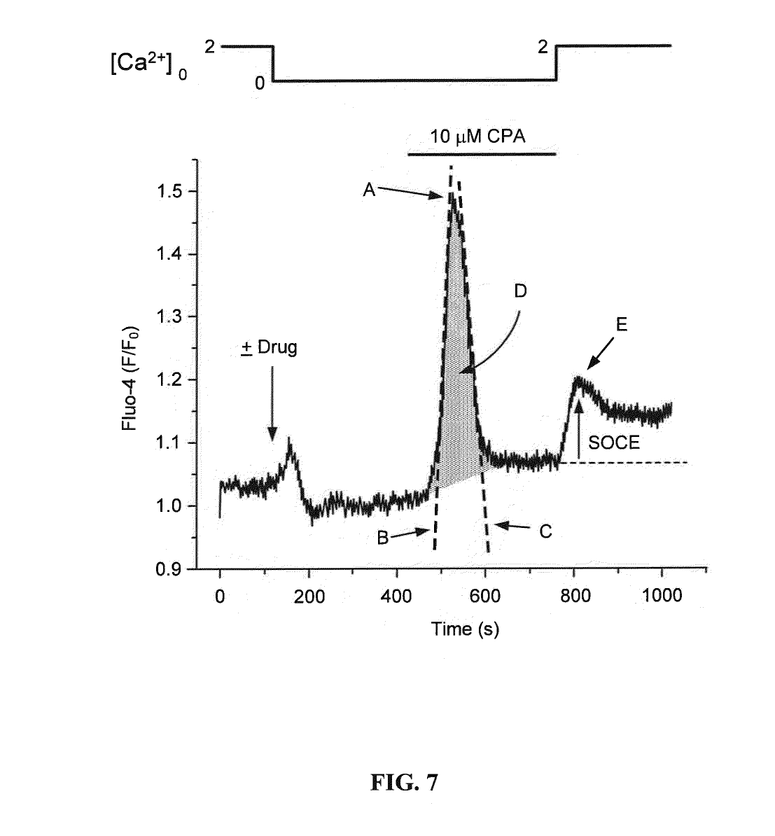

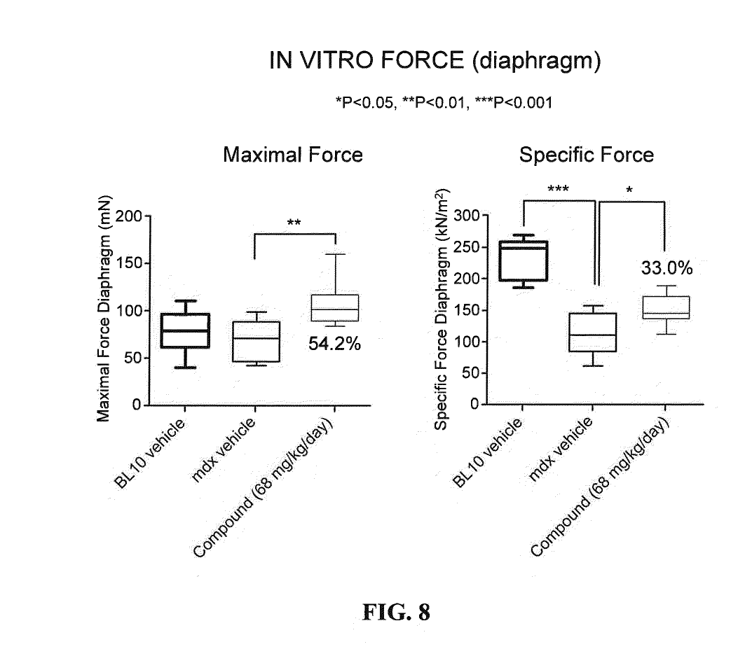

The definitions of Transient Measurements are shown in FIG. 3.

FIG. 4, shows the effect of Example 2 (10 uM, 30 uM), when applied to spontaneously beating cells in 4 mM calcium Tyrode's solution. Example 2 caused a dose-related reduction of proarrhythmia and calcium transient shortening.

FIG. 5 shows the effect of Example 2 (10 uM, 30 uM) which shortened CTD75 to 79% of control, with triangulation T75-25 reduced to 71% of control.

### Biological Example 3

### Analysis of Test Compounds on Calcium Release and Sarcoplasmic Reticulum Calcium Leak on Human Duchenne Muscular Dystrophy Myoblast Cells

Cell Preparation: De-identified telomerized human Duchenne muscular dystrophy (hDMD) myoblast cells were grown under standard cell culture conditions in 35 mm glass bottom petri dishes (MatTek, Ashland, Mass.) specially designed for perfusion and imaging.

Uncoated coverslips were coated with gelatin and hDMD cells seeded at ˜40-60% confluency 24 hours prior to fluorescence experiments.

Step 1. Cell Loading with the Fluorescent Ca²⁺ Indicator Fluo-4/AM (Cell Permeant Form): hDMD cells were loaded with 5 μM Fluo-4/AM (Thermo Fisher Scientific) for 45 min at room temperature in the same buffer used for experiments of which composition is described below. After the incubation, the cells are washed twice with the same buffer without the indicator. Fluorescence experiments were initiated after a 30 min incubation in indicator-free buffer to allow for de-esterification of the dye and conversion to its acid active form by endogenous esterases.

Step 2. Fluo-4 Fluorescence Detection: A 35 mm petri dish containing Fluo-4-loaded myoblasts was set on the stage of an inverted Nikon Diaphot 300 epifluorescence microscope equipped with a dual PMT ratiometric fluorimeter system (Model SFX-2, Solamere Technology Group, Salt Lake City, Utah) and optical switch (Model DX-1000, Salt Lake City, Utah), a B/W camera and monitor, a PC computer running on Windows 7 Pro operating system and A/D-D/A data acquisition hardware (Axon Instruments Inc., Digidata 1440 interface) and software (Axon Instruments Inc., Axoscope v. 9.2). During a typical experiment, ca. one third to one half of a cell (field of view narrowed by a manually-controlled diaphragm) is visualized on the B/W TV monitor with an oil immersion 40× Fluor objective (NA=1.3) and defines the area from which epifluorescence intensity will be measured by one of the two PMTs (non-ratiometric measurements). For all experiments, the cells are excited by visible light produced by a 100 W mercury arc lamp centered at 488 nm. Emitted fluorescence at 520 nm is transmitted to the lateral port of the microscope by means of dichroic mirrors and a specific barrier filter located in one of three microscope filter cubes. Before each day of experiments, background fluorescence is cancelled by measuring emitted fluorescence in an unloaded cell. For all cells loaded with Fluo-4/AM, relative fluorescence intensity at 520 nm (filtered at 5 Hz) is normalized to Fluo-4 fluorescence intensity measured in Ca²⁺-free medium (F/F0) prior to the addition of 10 μM cyclopiazonic acid (CPA; see protocol description below) to monitor Ca²⁺ leakage from the sarcoplasmic reticulum (SR). Free intracellular Ca²⁺ concentration is continuously monitored (>20 min; acquisition rate=200 Hz) with no evidence of photobleaching due to low level of excitation light intensity used and sensitivity of the Hamamatsu PMT set to high power.

Step 3. Composition of the physiological salt solutions (PSS) used in experiments are shown in the table:

Composition of the PSS

| Reagent | Concentration (mM) |
|---|---|
| NaCl | 135 |
| NaHCO3 | 10 |
| KCl | 4.2 |
| KH2PO4 | 1.2 |
| MgCl2 | 1.2 |
| D-Glucose | 5.5 |
| Hepes | 10 |
| CaCl2 | 2 or 0 |
| NaOH | pH-adjusted to 7.4 |

Step 4. FIG. 6 shows a typical experiment demonstrating the in vitro assay developed to evaluate Ca²⁺ release in a human DMD myoblast loaded with the fluorescent Ca²⁺ indicator Fluo-4/AM.

After mounting the petri dish containing Fluo-4-loaded cells on the microscope stage, cell perfusion with normal physiological salt solutions (PSS) containing 2 mM Ca²⁺ was initiated. A myoblast cell was then chosen under normal illumination. Fluo-4 fluorescence intensity recording was then initiated for 2 to 3 minutes to assess the level of loading and stability of the measurement. Once these conditions were met, the solution was switched to Ca²⁺-free PSS for 5 min, and then switched for another 5 min to Ca²⁺-free PSS containing only vehicle (Control; maximal concentration of 0.1% dimethyl sulfoxide) or vehicle plus drug to be tested (Test). The solution was then switched for 5-10 min to Ca²⁺-free PSS containing 10 μM cyclopiazonic acid (CPA; 10 μM), a specific inhibitor of the SR Ca²⁺-ATPase (SERCA), with

(Test) or without drug (Control). The application of this agent produces a slow transient rise in [Ca²⁺]i, that is the result of leakage of Ca²⁺ from the SR lumen to the cytoplasm which is then extruded outside the cell by the PMCA pump and/or the Na+/Ca²⁺ exchanger. This is a standard protocol to investigate store-operated Ca²⁺ entry in various cell types (Parekh and Putney, 2005) (FIG. 5)

Step 5. FIGS. 6B and 6C shows the parameters that are measured with this assay as end point measures:

a. FIG. 6B: (ΔF/F₀)/sec: Rate of Ca²⁺ release in CPA+Ca²⁺-free solution
b. FIG. 6B: Peak ΔF/F₀: Peak Ca²⁺ transient in CPA+Ca²⁺-free solution
c. FIG. 6B: (−ΔF/F₀)/sec: Rate of Ca²⁺ extrusion in CPA+Ca²⁺-free solution
d. FIG. 6C: Area under the Ca²⁺ transient in CPA+Ca²⁺-free solution as an index of total amount of Ca²⁺ released by the SR

FIG. 7 shows a typical experiment demonstrating the in vitro assay developed to evaluate Ca²⁺ release in a human DMD myoblast loaded with the fluorescent Ca²⁺ indicator Fluo-4/AM that includes the reintroduction of 2 mM Ca²⁺ at a later time point to highlight a further Ca²⁺ transit in the DMD myoblast (Peak labelled E in FIG. 7). The effect of a test compound on this SOCE calcium transit (CaT) is measured as an end point measure.

Results: The compounds of the present invention were tested as described above and the results are presented in Tables 1 and 2 below, utilizing experimental compound S107 as a control. The experimental compound, S107, has been used by various investigators (Lehnart et al., 2008) over recent years to highlight the potential of this compound to treat conditions associated with aberrant Ca²⁺ handling. For example, compound S107 has been shown to inhibit sarcoplasmic reticulum Ca²⁺ leak, reduce biochemical and histological evidence of muscle damage, improve muscle function and increase exercise performance in mdx mice (Bellinger et al., 2009). It has also been demonstrated that treating Sgcb−/− mice (sarcoglycan beta deficient mice; a murine model for type 2E human limb girdle muscular dystrophy) with the experimental S107 improved muscle specific force, calcium transients, and exercise capacity (Andersson et al., 2012). Treating aged mice with the experimental compound S107 reduced intracellular calcium leak, decreased reactive oxygen species, and enhanced tetanic Ca²⁺ release, muscle-specific force, and improved exercise capacity (Andersson et al., 2011). Experimental compound S107 is therefore a suitable control compound in assays that highlight the calcium modulating activity and therapeutic potential of compounds of the present invention.

TABLE 1

| | | | % Reduction in Peak |
|---|---|---|---|
| | | | CaT after return to |
| | | | 2 mM Ca²⁺ |
| Example | Concentration | n | (% Reduction in Peak E) |
| 9 | 30 uM | 3 | 40.7 |
| 2 | 30 uM | 4 | 41.4 |
| 13 | 30 uM | 4 | 80.8 |
| 17 | 30 uM | 4 | 21 |
| S107 | 30 uM | 12 | 52.8 |
| Control | | | |

TABLE 2

| | | | Integrated CPA-Induced CaT |
|---|---|---|---|
| | | | (area under the curve) |
| Example | Concentration | n | (% Reduction in Peak A) |
| 9 | 30 uM | 3 | 19.4 |
| 2 | 30 uM | 4 | 43.3 |
| 13 | 30 uM | 4 | 25.9 |
| 17 | 30 uM | 4 | 23.0 |
| S107 | 30 uM | 10 | 74.1 |
| Control | | | |

### Biological Example 4

### Muscular Dystrophy Model (Mdx Mice)

A naturally occurring dystrophin-deficient mutant mouse was first described in 1984 in a colony of C57BL/10 mice (C57BL/10ScSnJ) and has since been referred to as the “mdx-mouse” (Bulfield et al., 1984). This mouse, now called C57BL/10ScSn-Dmdmdx/J, is readily available from commercial breeders and widely used in basic and translational research. It carries a point mutation in exon 23 of the mouse dystrophin gene introducing a premature stop codon, which leads to the absence of full-length dystrophin. This type of mutation accounts for approximately one fifth of the mutations found in DMD patients.

The compounds of the invention can be tested using a reference drug of choice.

mdx C57BL/10 mice (15 mice per group) (Jackson Laboratory; mdx mice stock number 001801; C57BL/10 stock number 000476) were treated with Example 20 formulated in food (347 mg of Example 20 blended into 1 kg of Purina LabDiet Rodent 5001). Based on calculated food consumption, the dose of Example 20 was estimated to be 60 mg/Kg, administered daily in diet for 4 weeks.

Mice were delivered at 4 weeks of age, and acclimatized for an additional seven days prior to initiation of the study. All animals were weighed, and grouped into different treatment groups based on body weight. Each group received an appropriate daily dosage of Example 20 or vehicle. Age and sex-matched C57BL/10 (vehicle) were used as controls. Exposure levels of Example 1 (the metabolism product of Example 20) in mouse plasma was measured in a separate 5-day pharmacokinetic study in mdx mice prior to the initiation of the 4-week study. Plasma samples were also taken at the termination of the 4-week study for pharmacokinetic assessment in which exposure leves of Example 1 were again measured (the results are set out below). The functional measurements (grip strength measurement and exhaustion assay) were performed after treatment. At the end of the trial, the extensor digitorum longus muscle (EDL) and diaphragm in vitro force measurements were taken, and dissection of the mice was performed to collect tissues. Histology evaluation (H&E) was performed on the frozen sections of the diaphragm and gastrocnemius muscles for each group.

Preferred nitric oxide donating compounds of the current invention undergo a rapid and extensive first-pass metabolism to produce the parent calcium modulator and 1,4-butanediol mononitrate (a precursor of nitric oxide) after administration to an animal or person in need of such a compound. Thus, they are considered a nitric oxide releasing prodrug form of the calcium modulator. 1,4-Butanediol mononitrate is subsequently metabolized to NO and 1,4-butanediol.

To demonstrate this, Example 20 and Example 1 were incubated in mouse plasma and the amount of the parent compound was measured at various time points by LC/MS/MS analysis according to the following protocol.

The assay was carried out in 96-well microtiter plates. Compounds were incubated at 37° C. in the presence of the plasma. Reaction mixtures (50 μL) contained a final concentration of 20 μM test compound. The extent of metabolism was calculated as the disappearance of the test compound, compared to the 0-min control reaction incubations. Eucatropine was included as a positive control to verify assay performance.

At each of the time points, 500 μL of quench solution (100% acetonitrile with 0.1% formic acid) with internal standard was transferred to each well. Plates were sealed, vortexed, and centrifuged at 4° C. for 15 minutes at 4000 rpm. The supernatant was transferred to fresh plates for LC/MS/MS analysis.

All samples were analyzed on LC/MS/MS using an AB Sciex API 4000 instrument, coupled to a Shimadzu LC-20AD LC Pump system. Analytical samples were separated using a Waters Atlantis T3 dC18 reverse phase HPLC column (10 mm×2.1 mm) at a flow rate of 0.5 mL/min. The mobile phase consisted of 0.1% formic acid in water (solvent A) and 0.1% formic acid in 100% acetonitrile (solvent B). Elution conditions are detailed in the table below.

### Gradient Conditions:

| Time (min) | Flow (uL/min) | % A | % B |
|---|---|---|---|
| 0 | 500 | 98 | 2 |
| 0.3 | 500 | 98 | 2 |
| 1.4 | 500 | 2 | 98 |
| 2.0 | 500 | 2 | 98 |
| 2.01 | 500 | 98 | 2 |
| 2.5 | 500 | 98 | 2 |

Example 20 was shown to undergo a very rapid hydrolysis of the NO ester prodrug (Table 3) to produce the calcium modulator Example 1. Only, approximately 6% of Example 20 was detected after a 30-minute incubation. The result highlights the high plasma stability of calcium modulator Example 1 which was unchanged after 2-hours of incubation in plasma.

TABLE 3
Plasma Stability

| Compound | Time 0 min | Time 30 min | Time 60 min | Time 240 min |
|---|---|---|---|---|
| Eucatropine | 100.0% | 90.0% | 67.4% | 41.0% |
| Example 1 | 100.0% | 111.2% | 99.8% | 102.1% |
| Example 20 | 100.0% | 6.3% | 0.6% | 0.3% |

### Table 3, Analysis of the Plasma Stability of the Calcium Modulator and the NO-Prodrug Form of the Calcium Modulator.

### Assay to Determine Concentrations of Example 1 in Mouse Plasma Using LC/MS/MS.

Experiment Details: Each 20 μL of mouse plasma samples was first mixed with 100 μL methanol:acetonitrile (5:95 vol:vol) containing internal standard verapamil. The samples were vigorously vortexed for 15 minutes and then centrifuged for 15 minutes at 4000 rpm at 4° C. Finally, 50 μL of the extract was transferred to an injection plate and reconstituted with 70 μL of 0.1% formic acid in water for the injection to an LC/MS/MS system. The calibration standards for Example 1 were prepared by spiking the compound into the pre-dose plasma and processed in the same way as the samples. The LC-MS/MS analysis utilized positive electrospray ionizations under the multiple-reaction monitoring (MRM) mode for the detection of test article and the internal standard. The table below summarizes the LC/MS/MS conditions of the method used in the experiment.

LC/MS/MS Conditions

| | |
|---|---|
| HPLC | Shimadzu LC-20AD |
| Column Type | Thermo Hypersil Gold 50 × 2.1 mm 5μ |
| Mobile Phases | A: H2O with 0.1% FA; B: ACN with 0.1% FA |
| Pump Program | Gradient |
| Flow Rate | 350 uL/minute |

Gradient

| Time (min) | B (%) |
|---|---|
| 0 | 20 |
| 0.02 | 20 |
| 1.5 | 95 |
| 2.9 | 95 |
| 3 | 20 |
| 4.5 | Stop |

Analysis: Mass spec AB Sciex API5000
Results: A bioanalytical method was successfully established to measure Example 1 in mouse plasma. Oral administration of the NO-donating calcium modulator Example 20 (formulated in mouse chow) at an estimated dose of 60 mg/kg to mice afforded high plasma exposure of the parent calcium modulator Example 1 (>6500 ng/ml; 20 uM, over the 4-week period), in keeping with the results from the plasma stability study.

### Histology:

Diaphragm and gastrocnemius muscles of untreated and drug-treated mice were isolated and included in Killik frozen section medium, frozen and cut into 8-μm thick sections with the muscle fibers oriented transversely using a cryostat. Sections were stained with Hematoxylin & Eosin to evaluate markers of inflammation, muscle degeneration and regeneration and percentage central nuclei according to Grounds (TREAT NMD SOP protocol DMD_M.1.2.007, Quantification of histopathology in Haemotoxylin and Eosin stained muscle sections).

### In Vitro Force Measurement of Diaphragm Muscle:

Diaphragm muscles of untreated and drug-treated (Example 20) mice were isolated and tested according to Barton et al., (TREAT NMD SOP protocol DMD_M.1.2.002, Measuring isometric force of isolated mouse muscles in vitro). Treatment with Example 20 (ca. 60 mg/kg as described above) demonstrated a significant improvement in both maximal force and specific force in the diaphragm of mdx mice after four weeks of daily treatment (FIG. 8).

### Measurement of Muscle Regeneration and Markers of Inflammation:

Treatment of mdx mice with example 20 for 4-weeks resulted in a >25% increase in regenerating fibers/mm² in the diaphragm and >50% increase in regenerating fibers/mm² in the gastrocnemius muscle accompanied with >30% reduction in inflammatory foci/mm² in the gastrocnemius muscle.

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A compound having formula I: 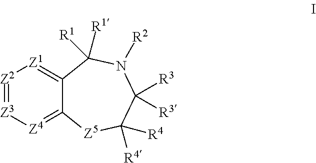
or a pharmaceutically acceptable salt, and including deuterated forms thereof, wherein:
Z¹ is —C(R⁸)— or —N—;
Z² is —C(R⁷)— or —N—;
Z³ is —C(R⁶)— or —N—;
Z⁴ is —C(R⁵)— or —N—;
Z⁵ is —O—, —S—, —S(O)—, —S(O)₂—, —NR^{x}— or —C(R^{x})₂—;
R¹, R^{1′}, R³, and R^{3′} are each independently selected from D, R^{x}, C(H)₂OR^{x}, C(H)₂OC(═O)R^{x}, C(═O)OR^{x}, C(═O)N(H)R^{x}, C(═O)R^{x}, and OC(═O)R^{x}; and optionally R¹ and R^{1′} taken together form oxo (═O); and optionally R³ and R^{3′} taken together form oxo (═O);
each of R⁵, R⁶, R⁷ and R⁸, which can be the same or different, are independently selected from H, D, halo, R^{x}, —OR^{x}, —SR^{x}, —N(R^{x})₂, —N(R^{x})C(═O)OR^{x}, —C(═O)N(R^{x})₂, —C(═O)OR^{x}, —C(═O)R^{x}, —OC(═O)R^{x}, —NO₂, —CN, —N₃, and —P(═O)(R^{x})₂; or
R⁵ and R⁶, together with the carbon atoms to which they are respectively attached, form an unsubstituted or substituted cycloalkyl or heterocyclic ring, wherein the substituents are one to three substituents independently selected from halo, aryl, Rx, hydroxyl nitro, amino, alkoxy, alkylthio, —CO₂H, and CN; or
R⁶ and R⁷, together with the carbon atoms to which they are respectively attached, form an unsubstituted or substituted cycloalkyl or heterocyclic ring, wherein the substituents are one to three substituents independently selected from halo, aryl, Rx, hydroxyl, nitro, amino, alkoxy, alkylthio, —CO₂H, and CN;
R² is -L¹-L²-G;
L¹ is —C(O)—, —C(O)C(O)— or —(C₁-C₆)alkyl optionally substituted with 1-3 halo; —(C₁-C₃)alkyl optionally substituted with 1-3 groups independently selected from halo and D; —(C₁-C₃)alkoxy optionally substituted with 1-3 groups independently selected from halo and D; or a spiro-(C₃-C₆)cycloalkyl optionally substituted with 1-2 groups independently selected from halo, D, methyl, and halogenated methyl;
L² is —O—, oxycarbonylaryl or oxycarbonylheteroaryl, wherein each aryl or heteroaryl group of L² is optionally substituted with one to three substituents independently selected from halo, D, —(C₁-C₆)alkyl, hydroxyl, nitro, amino, alkoxy, alkylthio, —CO₂H, and CN;
G is either absent or is one to three NO donors, provided that when G is absent, at least one of Z¹, Z², Z³ or Z⁴ is a nitrogen atom;
R⁴ and R^{4′} are each independently selected from H, D and R^{x}; or are combined to form oxo; or
R³ and R⁴ together with the carbon atoms to which they are respectively attached, form an unsubstituted or substituted cycloalkyl or heterocyclic ring, wherein the substituents are one to three substituents independently selected from halo, aryl, R^{x}, hydroxyl nitro, amino, alkoxy, alkylthio, —CO₂H, and CN;
each R^{x} is independently selected from H, D, alkyl, alkenyl, alkynyl, alkoxy, alkoxyalkyl, cycloalkyl, heterocyclyl, aryl, heteroaryl, cycloalkylalkyl, heterocyclylalkyl, arylalkyl, and heteroarylalkyl, wherein the alkyl, alkenyl or alkynyl portions of R^{x} can be optionally substituted with one to three substituents selected from halo, D, hydroxyl nitro, amino, —CO₂H and —CN.

2. The compound according to claim 1, wherein Z⁵ is —O—, —S—, —NR^{x}— or —C(R^{x})₂—.

3. The compound according to claim 1 having formula II: 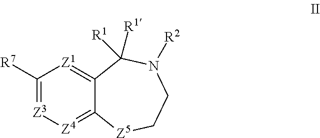
or a pharmaceutically acceptable salt, and including deuterated forms thereof, wherein:
Z¹ is —C(R⁸)— or —N—;
Z³ is —C(R⁶)— or —N—;
Z⁴ is —C(R⁵)— or —N—;
Z⁵ is —O—, —S—, —S(O)—, —S(O)₂—;
R¹ and R^{1′} are each independently selected from D and H;
each of R⁵, R⁶, and R⁸, are independently selected from H, D, halo, R^{x}, —OR^{x}, —SR^{x}, —N(R^{x})₂, —N(R^{x})C(═O)OR^{x}, —C(═O)N(R^{x})₂, —C(═O)OR^{x}, —C(═O)R^{x}, —OC(═O)R^{x}, —NO₂, —CN, —N₃, and —P(═O)(R^{x})₂; or
R⁵ and R⁶, together with the carbon atoms to which they are attached, form an unsubstituted or substituted cycloalkyl or heterocyclic ring, wherein the substituents are one to three substituents independently selected from halo, aryl, R^{x}, hydroxyl nitro, amino, alkoxy, alkylthio, —CO₂H and —CN;
R² is -L¹-L²-G;
L¹ is —C(O)—, —C(O)C(O)—, —(C₁-C₆)alkyl optionally substituted with 1-3 halo, —(C₁-C₃)alkyl optionally substituted with 1-3 groups independently selected from halo and D, —(C₁-C₃)alkoxy optionally substituted with 1-3 groups independently selected from halo and D, or a spiro-(C₃-C₆)cycloalkyl optionally substituted with 1-2 groups independently selected from halo, D, methyl, and halogenated methyl;
L² is —O—, oxycarbonylaryl or oxycarbonylheteroaryl, wherein each aryl or heteroaryl group of L² is optionally substituted with one to three substituents independently selected from halo, D, —(C₁-C₆)alkyl, hydroxyl, nitro, amino, alkoxy, alkylthio, —CO₂H and —CN;
R⁷ is selected from halo, D, R^{x}, —OR^{x}, —SR^{x}, —S(O)R^{x}, —S(O)₂R^{x}, —N(R^{x})₂, —N(R^{x})C(═O)OR^{x}, —C(═O)N(R^{x})₂, —C(═O)OR^{x}, —C(═O)R^{x}, —OC(═O)R^{x}, —NO₂, —CN, —N₃, and —P(═O)(R^{x})₂;
G is absent or is an NO donor, provided that when G is absent, at least one of Z¹, Z³ or Z⁴ is a nitrogen atom; and
each R^{x} is independently selected from H, D, alkyl, alkenyl, alkynyl, alkoxy, alkoxyalkyl, cycloalkyl, heterocyclyl, aryl, heteroaryl, cycloalkylalkyl, heterocyclylalkyl, arylalkyl, and heteroarylalkyl, wherein the alkyl, alkenyl and alkynyl portions of R^{x} are optionally substituted with one to three substituents selected from halo, D, hydroxyl, nitro, amino, alkoxy, alkylthio, —CO₂H and —CN.

4. The compound according to claim 3, wherein Z⁵ is —O— or —S—.

5. The compound according to claim 1, or a pharmaceutically acceptable salt, and including deuterated forms thereof, wherein:
G is absent or is an NO donor selected from —(C₁-C₁₀)alkyl substituted with 1 or 2 —ONO₂ groups, —C(H)₂—O—R⁹, —(C₁-C₆)alkylene-O—C(H)₂C(H)(ONO₂)—(C₁-C₆)alkyl, -phenylene-R⁹, —(C₁-C₆)alkylene-S(O)₂N(H)(OH), 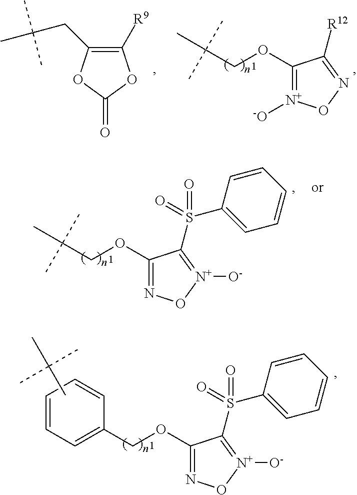
wherein each alkylene group of G is optionally substituted with one or more substituents selected from halo, aryl, hydroxyl, nitro, amino, alkoxy, alkylthio, —CO₂H, and CN;
R⁹ is —(C₂-C₁₀)alkyl substituted with 1 or 2 —ONO₂ groups;
R¹² is H or —(C₁-C₃)alkyl; and
n¹ is an integer from 2-5.

6. The compound according claim 1, having formula III: 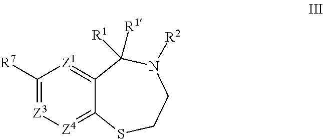
or a pharmaceutically acceptable salt, and including deuterated forms thereof, wherein:
Z¹ is —C(R⁸)— or —N—;
Z³ is —C(R⁶)— or —N—;
Z⁴ is —C(R⁵)— or —N—;
R¹ and R^{1′} are each independently selected from D or H;
each of R⁵, R⁶, and R⁸, which can be the same or different, are independently selected from H, D, halo, —(C₁-C₆)alkyl optionally substituted with 1-3 halo, —O—(C₁-C₆)alkyl optionally substituted with 1-3 halo, SRX, N(R^{x})₂, N(Rx)C(═O)OR^{x}, C(═O)N(R^{x})₂, C(═O)OR^{x}, C(═O)R^{x}, OC(═O)R^{x}, NO₂, —CN, and —N₃; or
R⁵ and R⁶, together with the carbon atoms to which they are attached, form an unsubstituted or substituted cycloalkyl or heterocyclic ring, wherein the substituents are one to three substituents independently selected from halo, R^{x}, hydroxyl, nitro, amino, alkoxy, alkylthio, —CO₂H and —CN;
R² is -L¹-L²-G;
L¹ is —C(O)—, —C(O)C(O)— or —(C₁-C₆)alkyl optionally substituted with 1-3 halo; —(C₁-C₃)alkyl optionally substituted with 1-3 groups independently selected from halo and D; —(C₁-C₃)alkoxy optionally substituted with 1-3 groups independently selected from halo and D; or a spiro-(C₃-C₆)cycloalkyl optionally substituted with 1-2 groups independently selected from halo, D, methyl, and halogenated methyl;
L² is —O—, oxycarbonylaryl or oxycarbonylheteroaryl, wherein each aryl or heteroaryl group of L² is optionally substituted with one to three substituents independently selected from halo, D, —(C₁-C₆)alkyl, hydroxyl, nitro, amino, alkoxy, alkylthio, —CO₂H and —CN;
R⁷ is selected from halo, D, R^{x}, —OR^{x}, —SR^{x}, —N(R^{x})₂, —N(R^{x})C(═O)OR^{x}, —C(═O)N(R^{x})₂, —C(═O)OR^{x}, —C(═O)R^{x}, —OC(═O)R^{x}, —NO₂, —CN, —N₃, and —P(═O)(R^{x})₂;
G is absent or is an NO donor selected from —(C₁-C₁₀)alkyl substituted with 1 or 2 —ONO₂ groups, —C(H)₂—O—(C₁-C₆)alkylene-O—C(H)₂C(H)(ONO₂)—(C₁-C₆)alkyl, -phenylene-R⁹, —(C₁-C₆)alkylene-S(O)₂N(H)(OH), 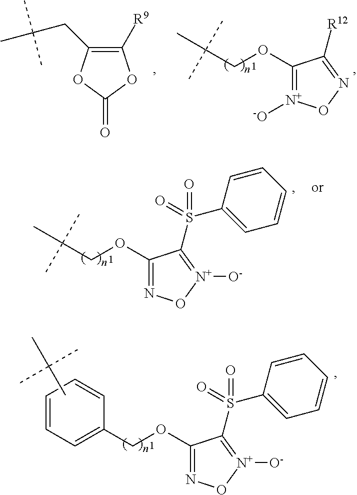
wherein each alkylene group of G is optionally substituted with one to three substituents independently selected from halo, aryl, hydroxyl, nitro, amino, alkoxy, alkylthio, —CO₂H and —CN, provided that when G is absent, at least one of Z¹, Z³ or Z⁴ is a nitrogen atom;
R⁹ is —(C₂-C₁₀)alkyl substituted with 1 or 2 —ONO₂ groups;
R¹² is H or —(C₁-C₃)alkyl;
n¹ is an integer from 0-5; and
each R^{x} is independently selected from H, D, alkyl, alkenyl, alkynyl, alkoxy, alkoxyalkyl, cycloalkyl, heterocyclyl, aryl, heteroaryl, cycloalkylalkyl, heterocyclylalkyl, arylalkyl, or heteroarylalkyl, wherein the alkyl, alkenyl or alkynyl portions of R^{x} are optionally substituted with one to three substituents independently selected from halo, D, hydroxyl, nitro, amino, alkoxy, alkylthio, —CO₂H and —CN.

7. The compound according to claim 1, or a pharmaceutically acceptable salt, and including deuterated forms thereof, wherein G is absent, R¹ and R^{1′} are each D, and one or both of Z¹ and Z³ are selected from —C(H)— or —N—, provided that at least one of Z¹ and Z³ is N.

8. The compound according to claim 1, or a pharmaceutically acceptable salt, and including deuterated forms thereof, wherein R⁷ is selected from halo, D, —O—C₁-C₄alkyl optionally substituted with one to three substituents independently selected from D and halo, —S—(C₁-C₄)alkyl optionally substituted with one to three substituents independently selected from D and halo, —S(O)—(C₁-C₄)alkyl optionally substituted with one to three substituents independently selected from D and halo, —S(O)₂—(C₁-C₄)alkyl optionally substituted with one to three substituents independently selected from D and halo, and —C(O)—(C₁-C₄)alkyl optionally substituted with one to three substituents independently selected from D and halo.

9. The compound according to claim 1 having a formula selected from IV(a), IV(b), IV(c), IV(d), IV(e) and IV(f): 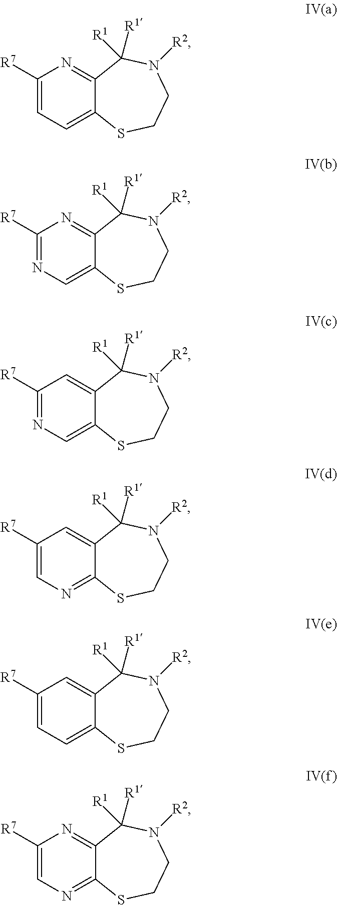
or a pharmaceutically acceptable salt, and including deuterated forms thereof, wherein:
R⁷ is —O—(C₁-C₄)alkyl optionally substituted with one to three substituents independently selected from D and halo, —(C₁-C₄)alkyl optionally substituted with one to three substituents independently selected from D and halo, and halo;
R² is -L¹-L²-G;
L¹ is —C(O)C(O)— or —C(R¹⁰)(R¹¹)—;
L² is —O— or oxycarbonylphenyl optionally substituted with 1-3 substituents independently selected from halo, D, aryl, —(C₁-C₃)alkyl, hydroxyl, nitro, amino, alkoxy, alkylthio, —CO₂H and —CN;
G is absent or is an NO donor selected from —(C₁-C₁₀)alkyl substituted with 1 or 2 —ONO₂ groups, —CH₂—O—R⁹, —(C₁-C₆)alkylene-O—CH₂CH(ONO₂)—(C₁-C₆)alkyl, -phenylene-R⁹, —(C₁-C₆)alkylene-S(O)₂NH(OH), 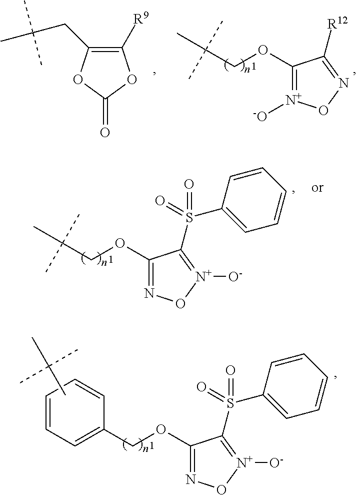
provided that when G is absent, the compound is other than Formula IV(e);
R⁹ is —(C₂-C₁₀)alkyl substituted with 1 or 2 —ONO₂ groups;
R¹⁰ and R¹¹ are each independently selected from H, D, —CH₃, halogenated methyl, —CD₃, or R¹⁰ and R¹¹ taken together with the carbon to which they are attached join to form a spiro-(C₃-C₆)cycloalkyl optionally substituted with 1-2 groups selected from halo, D, methyl, and halogenated methyl;
R¹² is H or —(C₁-C₃)alkyl; and
n¹ is an integer from 0-3,
wherein each alkylene group of G is optionally substituted with 1-2 substituents selected from halo, aryl, hydroxyl, nitro, amino, alkoxy, alkylthio, —CO₂H and —CN.

10. The compound according to claim 1, wherein:
R⁷ is selected from —OCH₃, —OCD₃, —OCF₃, —O-n-propyl, —O-isopropyl, —O-n-butyl, —O-sec-butyl, —O-t-butyl, —O-isobutyl, —O-cylclopropyl, —CD₃ and —CF₃.

11. The compound according to claim 1 having a formulae selected from V(a), V(b), V(c), V(d), V(e), V(f), V(g), V(h), V(i), V(j), V(k), or V(l): 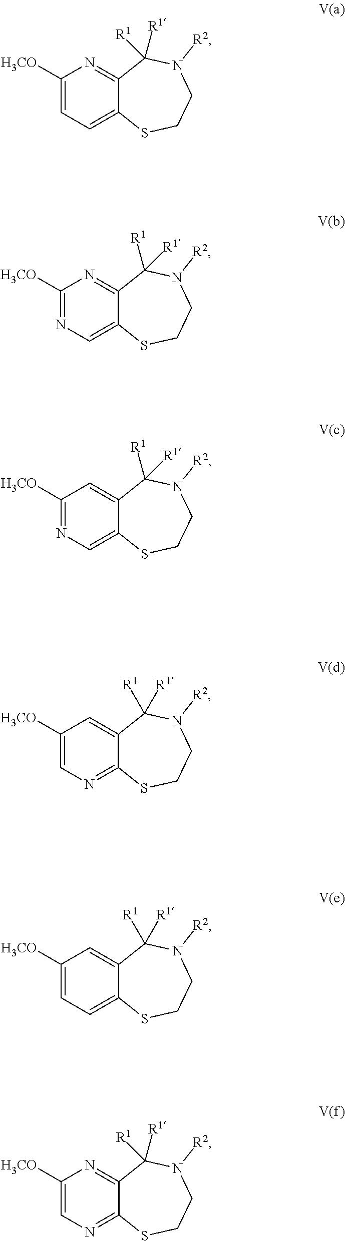 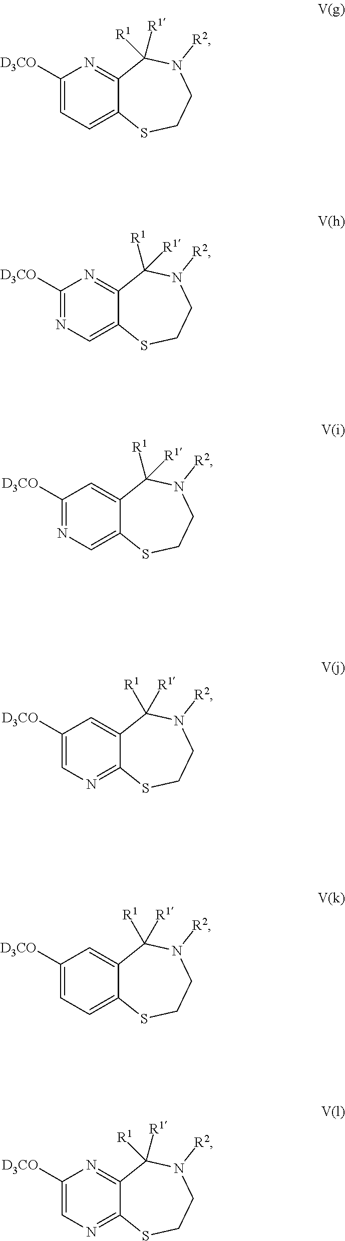
or a pharmaceutically acceptable salt, and including deuterated forms thereof, wherein:
R¹ and R^{1′} are each independently selected from D and H;
R² is -L¹-L²-G;
L¹ is —C(O)C(O)— or —C(R¹⁰)(R¹¹)—;
L² is —O—, oxycarbonylaryl or oxycarbonylheteroaryl, wherein the aryl or heteroaryl portions are optionally substituted with 1-2 substituents independently selected from halo, —(C₁-C₃)alkyl, hydroxyl, nitro, amino, alkoxy, alkylthio, —CO₂H, and CN;
G is absent or is an NO donor selected from —(C₁-C₁₀)alkyl substituted with 1 or 2 —ONO₂ groups, —CH₂—O—R⁹, —(C₁-C₆)alkylene-O—CH₂CH(ONO₂)—(C₁-C₆)alkyl, -phenylene-R⁹, —(C₁-C₆)alkylene-S(O)₂NH(OH), 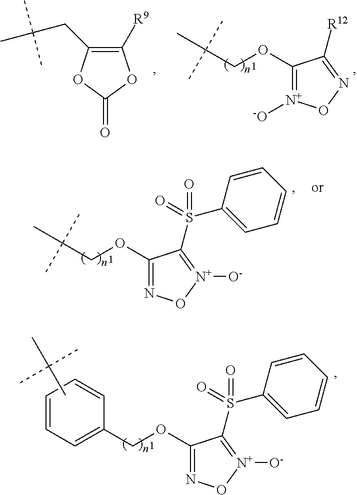
provided that when G is absent, the compound has a formula other than V(e);
R⁹ is —(C₂-C₁₀)alkyl substituted with 1 or 2 —ONO₂ groups;
R¹⁰ and R¹¹ are each independently selected from H, D, —CH₃, halogenated methyl and —CD₃, or R¹⁰ and R¹¹ taken together with the carbon to which they are attached join to form a (C₃-C₆)cycloalklyl optionally substituted with 1-2 groups selected from halo, D, methyl, and halogenated methyl;
R¹² is H or —(C₁-C₃)alkyl; and
n¹ is an integer from 0-3;
wherein each alkylene group of G is optionally substituted with 1-2 substituents selected from halo, aryl, hydroxyl, amino, alkoxy, and alkylthio.

12. The compound according to claim 1, wherein
R² is 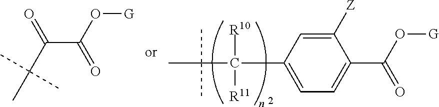
G is an NO donor selected C₁₋₁₀alkyl substituted with 1 or 2 —ONO₂ or 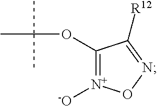
R¹² is H or CH₃;
R¹⁰ and R¹¹ are each independently selected from H, D, —CH₃, halogenated methyl, and —CD₃; or R¹⁰ and R¹¹ taken together with the carbon to which they are attached join to form a cyclopropyl;
Z is H, halo or —(C₁-C₃)alkoxy, and
n² is an integer from 1-2.

13. The compound according to claim 1, wherein:
R² is 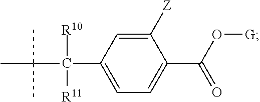
and
R¹⁰ and R¹¹ are each independently selected from H, D, —CH₃, halogenated methyl, and —CD₃, or R¹⁰ and R¹¹ taken together with the carbon to which they are attached join to form a cyclopropyl;
G is absent or C₁₋₁₀alkyl substituted with 1 or 2 —ONO₂; and
Z is H, fluoro or methoxy.

14. The compound according to claim 1, wherein:
R² is 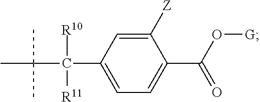
and
R¹⁰ and R¹¹ are each independently selected from H, D, —CH₃, halogenated methyl, and —CD₃, or R¹⁰ and R¹¹ taken together with the carbon to which they are attached join to form a cyclopropyl;
G is absent or C₁₋₁₀alkyl substituted with 1 or 2 —ONO₂; and
Z is fluoro or methoxy.

15. The compound according to claim 1, wherein:
R² is 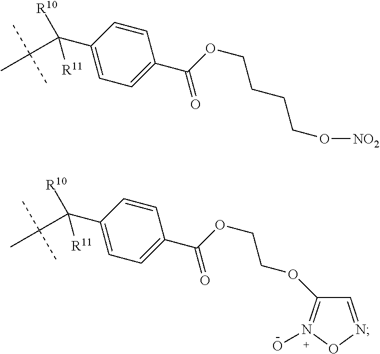
and R¹⁰ and R¹¹ are each independently selected from H, D, —CH₃, and —CD₃; or R¹⁰ and R¹¹ join together to form a cyclopropyl.

16. A compound according to claim 1 selected from the group consisting of: 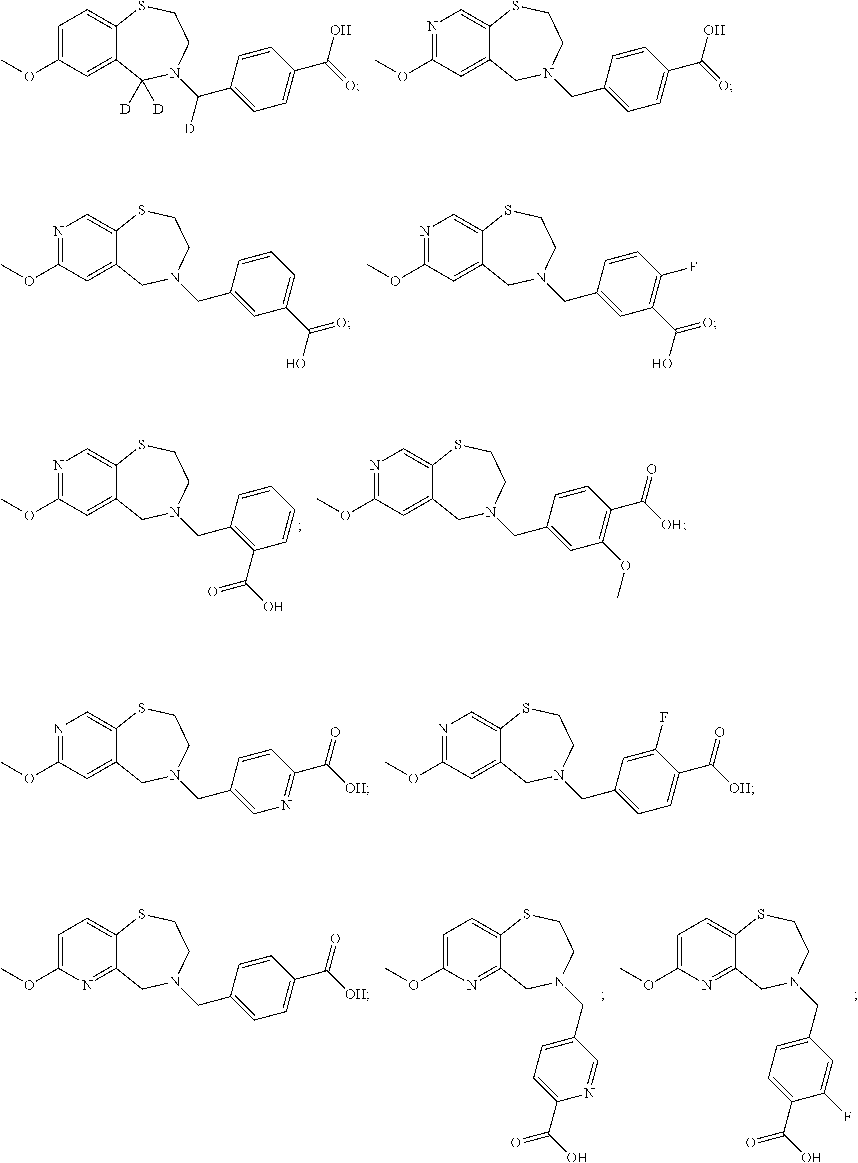 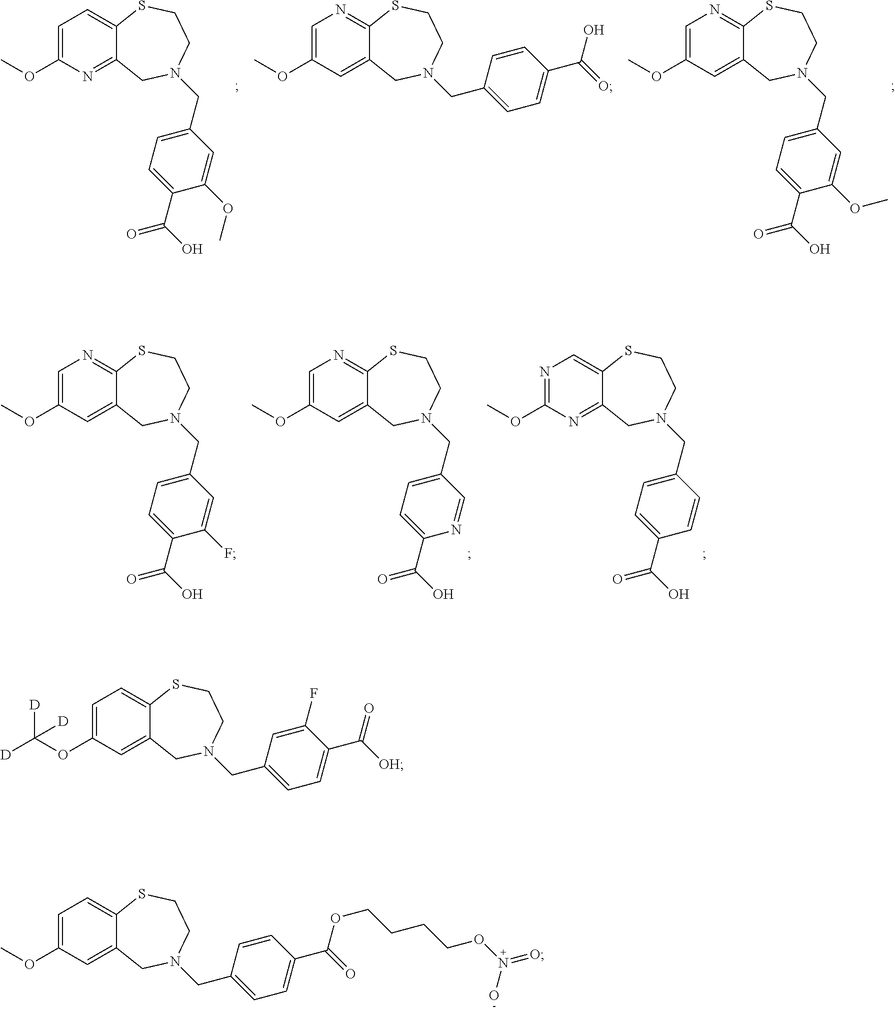 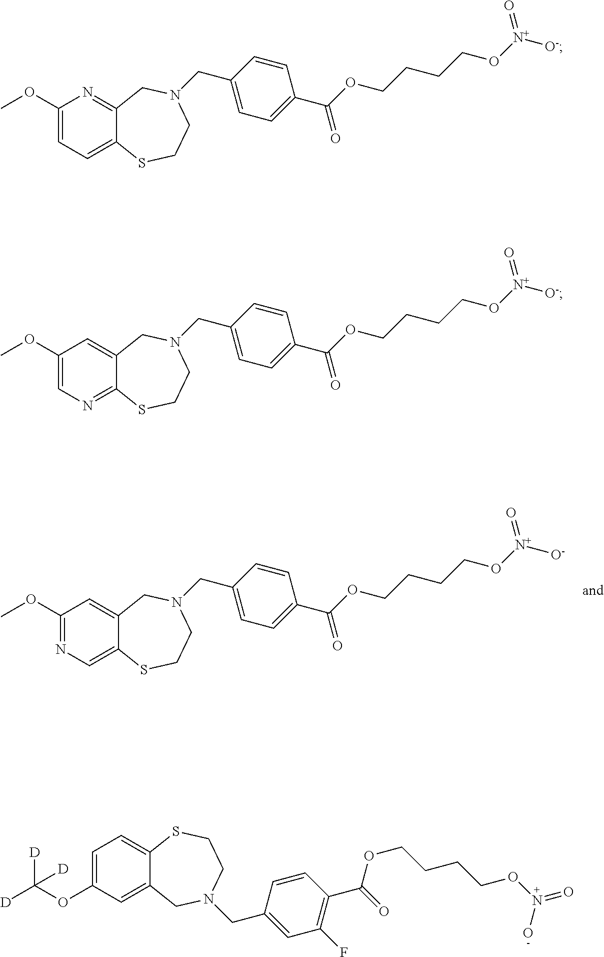
or a pharmaceutically acceptable salt of any of the above compounds, including deuterated forms thereof.

17. The compound according to claim 1, wherein the salt is selected from sodium, potassium, magnesium, hemifumarate, hydrochloride or hydrobromide.

18. A pharmaceutical composition comprising a compound according to claim 1, in combination with one or more pharmaceutically acceptable excipients or carriers.

19. A pharmaceutical composition comprising a compound according to claim 1, in combination with one or more NO donors and optionally with one or more pharmaceutically acceptable excipients or carriers.

20. A method of treating or preventing muscle disorders, diseases and conditions associated with dysfunctions in calcium homeostasis or modulation, comprising administering to a subject in need of such treatment an amount of a compound of claim 1.

21. A method of treating or preventing a condition selected from cardiac disorders and diseases, muscle fatigue, musculoskeletal disorders and diseases, diseases associated with colon function, CNS disorders and diseases, cognitive dysfunction, neuromuscular disorders and diseases, bone disorders and diseases, cancer cachexia, malignant hyperthermia, diabetes, sudden cardiac death, and sudden infant death syndrome, or for improving cognitive function, the method comprising administering to a subject in need thereof a therapeutically effective amount of a compound of claim 1, optionally in combination with an NO donor, to effectuate such treatment.

22. The method according to claim 21, wherein the condition is associated with an abnormal function of a calcium homeostasis or modulation.

23. The method according to claim 21, wherein the cardiac disorders and diseases are selected from irregular heartbeat disorders, atrial and ventricular arrhythmia, atrial and ventricular fibrillation, atrial and ventricular tachyarrhythmia, atrial and ventricular tachycardia, catecholaminergic polymorphic ventricular tachycardia (CPVT), exercise-induced irregular heartbeat disorders and diseases, congestive heart failure, chronic heart failure, acute heart failure, systolic heart failure, diastolic heart failure, acute decompensated heart failure, cardiac ischemia/reperfusion (FR) injury, chronic obstructive pulmonary disease, FR injury following coronary angioplasty or following thrombolysis for the treatment of myocardial infarction (MI), or high blood pressure.

24. The method according to claim 21, wherein the musculoskeletal disorder, disease or condition is selected from exercise-induced skeletal muscle fatigue, a congenital myopathy, Duchenne Muscular Dystrophy (DMD), Becker's Muscular Dystrophy (BMD), Limb-Girdle Muscular Dystrophy (LGMD), facioscapulohumeral dystrophy, myotonic muscular dystrophy, congenital muscular dystrophy (CMD), distal muscular dystrophy, Emery-Dreifuss muscular dystrophy, oculopharyngeal muscular dystrophy, spinal muscular atrophy (SMA), Spinal and bulbar muscular atrophy (SBMA), age-related muscle fatigue, sarcopenia, central core disease; bladder disorders, orincontinence.

25. The method according claim 1, wherein the CNS disorders and diseases are selected from Alzheimer's Disease (AD), neuropathy, seizures, Parkinson's Disease (PD), or Huntington's Disease (HD); and the neuromuscular disorders and diseases are selected from Spinocerebellar ataxia (SCA), or Amyotrophic lateral sclerosis (ALS, Lou Gehrig's disease).

26. A method for treating a subject that has Duchenne Muscular Dystrophy, comprising the step of administering to said subject an amount of a compound of claim 1, in combination with an antisense oligonucleotide (AO) which is specific for a splicing sequence of at least one exon of the DMD gene; a steroid such as prednisone, deflazacort or the like; a myostatin (GDF-8) antibody (e.g. PF-06252616, BMS-986089, LY2495655 or the like; folliststin gene therapy; micro and mini dystrophin gene (AAV) therapy; micro and mini utrophin gene (AAV) therapy; an upregulator of utrophin expression such as SMT C1100 and the like; anti-fibrotic agents such as halofuginone, FG-3019, BG00011 (STX-100) and the like; a stop-codon (or nonsence) readthrough agent such as PTC124, ataluren, aminoglycoside antibiotics and the like, or human growth factor.

27. The method according to claim 26, wherein the splicing sequence is of exon 23, 45, 44, 50, 51, 52 and/or 53 of the DMD gene.
